(19) United States
(12) Patent Application Publication
(10) Pub. No.: US 2024/0417382 A1
(43) Pub. Date: Dec. 19, 2024

(54) SALTS OF A HETEROCYCLIC COMPOUND AND CRYSTALLINE FORMS, PROCESSES FOR PREPARING, THERAPEUTIC USES, AND PHARMACEUTICAL COMPOSITIONS THEREOF

(71) Applicant: Sumitomo Pharma America, Inc., Marlborough, MA (US)

(72) Inventors: Nandkumar Nivritti BHOGLE, North Reading, MA (US); Takahiko HASHIZUKA, Osaka (JP); Robert Joseph PRYTKO, Millbury, MA (US); John R. SNOONIAN, Bolton, MA (US); Harold Scott WILKINSON, Westboro, MA (US); Haitao ZHANG, Shrewsbury, MA (US)

(73) Assignee: Sumitomo Pharma America, Inc., Marlborough, MA (US)

(21) Appl. No.: 18/642,933

(22) Filed: Apr. 23, 2024

Related U.S. Application Data
(63) Continuation of application No. 17/347,709, filed on Jun. 15, 2021, now abandoned.
Application No. 17/347,709 is a continuation of application No. 16/817,749, filed on Mar. 13, 2020, now Pat. No. 11,136,304.
(60) Provisional application No. 62/818,256, filed on Mar. 14, 2019.

(51) Int. Cl. C07D 311/76

(52) U.S. Cl. CPC C07D 311/76; C07B 2200/13

(57) ABSTRACT

The present disclosure relates to salts of (R)-1-(8-fluoroisochroman-1-yl)-N-methylmethanamine, crystalline forms thereof, and methods of preparation thereof, which are useful in the treatment of CNS disorders.

### CROSS REFERENCE TO RELATED APPLICATIONS

This application claims priority from U.S. provisional application 62/818,256, filed Mar. 14, 2019, the entire disclosure of which is hereby incorporated herein by reference.

### FIELD

This application relates to salts of (R)-1-(8-fluoroisochroman-1-yl)-N-methylmethanamine (Compound 1), and crystalline forms, processes for preparing, therapeutic uses, and pharmaceutical compositions thereof.

### BACKGROUND

Central nervous system diseases and disorders affect a wide range of the population with differing severity. Neurological and psychiatric diseases and disorders include major depression, schizophrenia, bipolar disorder, obsessive compulsive disorder (OCD), panic disorder, and posttraumatic stress disorder (PTSD), among others. These diseases and disorders affect a person's thoughts, mood, behavior and social interactions and can significantly impair daily functioning. See, e.g., Diagnostic and Statistical Manual of Mental Disorders, 4^{th} Ed., American Psychiatric Association (2000) (“DSM-IV-TR”); Diagnostic and Statistical Manual of Mental Disorders, 5^{th} Ed., American Psychiatric Association (2013) (“DSM-5”). Furthermore, neuropsychiatric symptoms such as apathy, depression, anxiety, cognitive impairment, psychosis, aggression, agitation, impulse control and sleep disruption are now recognized as core impairments of neurological diseases and disorders such as Alzheimer's and Parkinson's diseases.

Various drugs are currently being developed for the treatment of CNS disorders. For example, (R)-1-(8-fluoroisochroman-1-yl)-N-methylmethanamine, which is reported in U.S. Pat. No. 10,196,403, the entirety of which is incorporated herein by reference, is useful in the treatment of CNS disorders. There is a need for salts and new forms of (R)-1-(8-fluoroisochroman-1-yl)-N-methylmethanamine for facilitating the manufacture of safe, effective, and high quality drug products.

### SUMMARY

Provided herein are salts of (R)-1-(8-fluoroisochroman-1-yl)-N-methylmethanamine and crystalline forms thereof. Compound (R)-1-(8-fluoroisochroman-1-yl)-N-methylmethanamine (Compound 1) has the following structure:

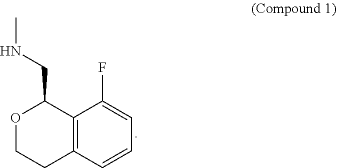

In some embodiments, provided are processes of preparing (R)-1-(8-fluoroisochroman-1-yl)-N-methylmethanamine (Compound 1), or salts or crystalline forms thereof.

In some embodiments, provided are methods of using (R)-1-(8-fluoroisochroman-1-yl)-N-methylmethanamine (Compound 1), or salts or crystalline forms thereof, in the treatment of CNS disorders.

In some embodiments, provided are pharmaceutical compositions comprising (R)-1-(8-fluoroisochroman-1-yl)-N-methylmethanamine (Compound 1), or salts or crystalline forms thereof, as described herein, and one or more pharmaceutically acceptable excipients.

### BRIEF DESCRIPTION OF THE DRAWINGS

FIG. 1 shows an X-ray powder diffraction (XRPD) pattern of Compound 1 Hydrochloride Form HA.

FIG. 2 shows a differential scanning calorimetry (DSC) thermogram of Compound 1 Hydrochloride Form HA.

FIG. 3 shows a thermogravimetric analysis (TGA) thermogram of Compound 1 Hydrochloride Form HA.

FIG. 4 shows a dynamic vapor sorption (DVS) isotherm of Compound 1 Hydrochloride Form HA.

FIG. 5 shows an XRPD pattern of Compound 1 Hydrochloride Form HB.

FIG. 6 shows an XRPD pattern of Compound 1 Phosphate.

FIG. 7 shows a DSC thermogram of Compound 1 Phosphate.

FIG. 8 shows a TGA thermogram of Compound 1 Phosphate.

FIG. 9 shows a DVS isotherm of Compound 1 Phosphate.

FIG. 10 shows an XRPD pattern of Compound 1 L-Tartrate Form LA.

FIG. 11 shows a DSC thermogram of Compound 1 L-Tartrate Form LA.

FIG. 12 shows a TGA thermogram of Compound 1 L-Tartrate Form LA.

FIG. 13 shows a DVS isotherm of Compound 1 L-Tartrate Form LA.

FIG. 14 shows an XRPD pattern of Compound 1 L-Tartrate Form LB.

FIG. 15 shows a DVS isotherm of Compound 1 L-Tartrate Form LB.

FIG. 16 shows an XRPD pattern of Compound 1 L-Tartrate Form LC.

FIG. 17 shows a DSC thermogram of Compound 1 L-Tartrate Form LC.

FIG. 18 shows a TGA thermogram of Compound 1 L-Tartrate Form LC.

FIG. 19 shows a DVS isotherm of Compound 1 L-Tartrate Form LC.

FIG. 20 shows an XRPD pattern of Compound 1 D-Tartrate.

FIG. 21 shows a DVS isotherm of Compound 1 D-Tartrate.

FIG. 22 shows an XRPD pattern of Compound 1 Fumarate Form FA.

FIG. 23 shows a DSC thermogram of Compound 1 Fumarate Form FA.

FIG. 24 shows a TGA thermogram of Compound 1 Fumarate Form FA.

FIG. 25 shows a DVS isotherm of Compound 1 Fumarate Form FA.

FIG. 26 shows an XRPD pattern of Compound 1 Fumarate Form FB.

FIG. 27 shows a DSC thermogram of Compound 1 Fumarate Form FB.

FIG. 28 shows a TGA thermogram of Compound 1 Fumarate Form FB.

FIG. 29 shows a DVS isotherm of Compound 1 Fumarate Form FB.

FIG. 30 shows an XRPD pattern of Compound 1 Citrate.

FIG. 31 shows a DSC thermogram of Compound 1 Citrate.

FIG. 32 shows a TGA thermogram of Compound 1 Citrate.

FIG. 33 shows a DVS isotherm of Compound 1 Citrate.

FIG. 34 shows an XRPD pattern of Compound 1 Succinate.

FIG. 35 shows a DSC thermogram of Compound 1 Succinate.

FIG. 36 shows a TGA thermogram of Compound 1 Succinate.

FIG. 37 shows a DVS isotherm of Compound 1 Succinate.

FIG. 38 shows an XRPD pattern of Compound 1 Glutarate.

FIG. 39 shows a DVS isotherm of Compound 1 Glutarate.

FIG. 40 shows an XRPD pattern of Compound 1 L-Malate.

FIG. 41 shows a DSC thermogram of Compound 1 L-Malate.

FIG. 42 shows a TGA thermogram of Compound 1 L-Malate.

FIG. 43 shows a DVS isotherm of Compound 1 L-Malate.

FIG. 44 shows an XRPD pattern of Compound 1 Besylate.

FIG. 45 shows a DSC thermogram of Compound 1 Besylate.

FIG. 46 shows a TGA thermogram of Compound 1 Besylate.

FIG. 47 shows a DVS isotherm of Compound 1 Besylate.

### DETAILED DESCRIPTION

The methods of the disclosure relate to the use of compounds and compositions disclosed herein to treat neurological or psychiatric diseases, disorders or impairments. In some embodiments, the neurological or psychiatric disease or disorder is depression, bipolar disorder, pain, schizophrenia, obsessive compulsive disorder, psychostimulation, addiction, social disorder, attention deficit hyperactivity disorder, an anxiety disorder, a movement disorder, epilepsy, autism, Alzheimer's disease, Parkinson's disease or cognitive impairments. In one embodiment, the disease or disorder is depression, particularly treatment-resistant depression (TRD), major depressive disorder (MDD), unipolar depression, bipolar depression or depression associated with another disease or disorder. In some embodiments, the impairments in neurological diseases or disorders such as Alzheimer's and Parkinson's diseases include neuropsychiatric symptoms such as apathy, depression, anxiety, cognitive impairment, psychosis, aggression, agitation, impulse control disorders, and/or sleep disorders.

The description herein sets forth details to provide an understanding of various embodiments of the disclosure, and is made with the understanding that the provided disclosures are an exemplification of the claimed subject matter without intending to limit the claims to specific embodiments. Accordingly, specific embodiments disclosed herein may be combined with other specific embodiments disclosed herein, including specific embodiments under various headings, which are provided for convenience and organization, but are not to be construed to limit the claims in any way.

All published documents cited herein are hereby incorporated by reference in their entirety.

### Definitions

Unless defined otherwise, all technical and scientific terms used herein have the same meaning as commonly understood by one of ordinary skill in the art.

As used herein, the singular forms “a”, “an” and “the” are intended to include the plural forms as well, unless the context clearly indicates otherwise.

As used herein, and unless otherwise specified, the term “about”, when used in connection with a numeric value or range of values which is provided to describe a particular solid form (e.g., a specific temperature or temperature range, such as describing a melting, dehydration, or glass transition; a mass change, such as a mass change as a function of temperature or humidity; a solvent or water content, in terms of, for example, mass or a percentage; or a peak position, such as in analysis by, for example, ¹³C NMR, DSC, TGA and XRPD), indicate that the value or range of values may deviate to an extent deemed reasonable to one of ordinary skill in the art while still describing the particular solid form. Specifically, the term “about”, when used in this context, indicates that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values while still describing the particular solid form. In some embodiments, the values can vary by about 5%. The term “about”, when used in reference to a degree 2-theta value refers to ±0.3 degrees 2-theta or ±0.2 degrees 2-theta. In some embodiments, “about” refers to a degree 2-theta value of ±0.2 degrees 2-theta. In some embodiments, “about” refers to a temperature of ±3° C.

As used herein, the phrase “alkali metal bicarbonate,” employed alone or in combination with other terms, refers to a base having formula M(HCO₃), wherein M refers to an alkali metal (e.g. lithium, sodium, or potassium). Example alkali metal bicarbonate include, but are not limited to, lithium bicarbonate, sodium bicarbonate, and potassium bicarbonate.

As used herein, the phrase “alkali metal alkoxide,” employed alone or in combination with other terms, refers to a base having formula M(O-alkyl), wherein M refers to an alkali metal (e.g. lithium, sodium, or potassium). Examples alkali metal alkoxide include, but are not limited to lithium alkoxide, sodium alkoxide, and potassium alkoxide.

As used herein, the phrase “metal hydroxide base,” employed alone or in combination with other terms, refers to a base having formula MOH, wherein M refers to a metal such as an alkali metal (e.g. lithium, sodium, or potassium). Example alkali metal hydroxide bases include, but are not limited to lithium hydroxide, sodium hydroxide, and potassium hydroxide.

As used herein, the terms “comprising” and “including” or grammatical variants thereof are to be taken as specifying the stated features, integers, steps or components but do not preclude the addition of one or more additional features, integers, steps, components or groups thereof. These terms encompass the term “consisting of”.

The expressions, “ambient temperature” and “room temperature,” as used herein, are understood in the art, and refer generally to a temperature, e.g., a reaction temperature, that is about the temperature of the room in which the reaction is carried out, for example, a temperature from about 20° C. to about 30° C.

As used herein, the term “amorphous” or “amorphous form” is intended to mean that the substance, component, or product in question is not crystalline as determined, for instance, by XRPD or where the substance, component, or product in question, for example is not birefringent when viewed microscopically. For example, amorphous means essentially without regularly repeating arrangement of molecules or lacks the long range order of a crystal, i.e., amorphous form is non-crystalline. An amorphous form does not display a defined x-ray diffraction pattern with sharp maxima. In certain embodiments, a sample comprising an amorphous form of a substance can be substantially free of other amorphous forms and/or crystalline forms. For example, an amorphous substance can be identified by an XRPD spectrum having an absence of readily distinguishable reflections.

As used herein, the term “chemical purity” or “purity” refers to a measurement of purity compound. In some embodiments, the compound described herein can be isolated with a purity of at least about 80%, about 85%, about 90%, about 95%, about 96%, about 97%, about 98%, or about 99%. In some embodiments, the compound described herein can be isolated with an enantiomeric purity greater than about 90%. In some embodiments, the compound described herein can be isolated with an enantiomeric purity greater than about 95%. In some embodiments, the compound described herein can be isolated with an enantiomeric purity greater than about 99%. The measurement can be determined by methods well-known in the art, e.g., by elemental analysis, column chromatography, NMR spectroscopy, and the like.

As used herein, the term “crystalline” or “crystalline form” refers to a crystalline solid form of a chemical compound, including, but not limited to, a single-component or multiple-component crystal form, e.g., including solvates, hydrates, clathrates, and a co-crystal. For example, crystalline means having a regularly repeating and/or ordered arrangement of molecules, and possessing a distinguishable crystal lattice. The term “crystalline form” is meant to refer to a certain lattice configuration of a crystalline substance. Different crystalline forms of the same substance typically have different crystalline lattices (e.g., unit cells), typically have different physical properties attributed to their different crystalline lattices, and in some instances, have different water or solvent content. The different crystalline lattices can be identified by solid state characterization methods such as by X-ray powder diffraction (XRPD). Other characterization methods such as differential scanning calorimetry (DSC), thermogravimetric analysis (TGA), dynamic vapor sorption (DVS), and the like further help identify the crystalline form as well as help determine stability and solvent/water content.

As used herein, the term “% crystallinity” or “crystalline purity,” means percentage of a crystalline form in a preparation or sample, which may contain other forms such as an amorphous form of the same compound, or at least one other crystalline form of the compound, or mixtures thereof. In some embodiments, the crystalline forms can be isolated with a purity of at least about 80%, about 85%, about 90%, about 95%, about 96%, about 97%, about 98%, or about 99%. In some embodiments, the crystalline forms can be isolated with a purity greater than about 90%. In some embodiments, the crystalline forms can be isolated with a purity greater than about 95%. In some embodiments, the crystalline forms can be isolated with a purity greater than about 99%.

As used herein, “delaying” development of a disorder mean to defer, hinder, slow, stabilize, and/or postpone development of the disorder. Delay can be of varying lengths of time, depending on the history of the disease and/or the individual being treated.

As used herein, the term “disorder” or specifically identified disorders disclosed herein, (e.g. CNS disorders) refer to the disorder as defined in the Diagnostic and Statistical Manual of Mental Disorders, Fifth Edition (DSM-5).

As used herein, the term “enantiomeric purity” refers to a measurement of purity for a chiral compound. In some embodiments, the compound described herein can be isolated with an enantiomeric purity of at least about 80%, about 85%, about 90%, about 95%, about 96%, about 97%, about 98%, or about 99%. In some embodiments, the compound described herein can be isolated with an enantiomeric purity greater than about 99%. In some embodiments, the compound described herein can be isolated with an enantiomeric purity greater than about 90%. In some embodiments, the compound described herein can be isolated with an enantiomeric purity greater than about 95%. The measurement can be determined by methods well-known in the art, e.g., by specific optical rotation, chiral column chromatography, NMR spectroscopy, and the like.

The term “hydrate,” as used herein, is meant to refer to a solid form (e.g., crystalline form) of Compound 1 and its salts that includes water. The water in a hydrate can be present in a stoichiometric amount with respect to the amount of salt in the solid, or can be present in varying amounts, such as can be found in connection with channel hydrates.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas. As used herein, the term “organic solvent” refers to carbon-based solvents (i.e., they contain carbon in their structure) that are employed to dissolve or disperse one or more compounds described herein.

Suitable solvents can include halogenated solvents such as carbon tetrachloride, bromodichloromethane, dibromochloromethane, bromoform, chloroform, bromochloromethane, dibromomethane, butyl chloride, dichloromethane (methylene chloride), tetrachloroethylene, trichloroethylene, 1,1,1-trichloroethane, 1,1,2-trichloroethane, 1,1-dichloroethane, 2-chloropropane, 1,1,1-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

Suitable solvents can include ether solvents include dimethoxymethane, tetrahydrofuran, 1,3-dioxane, 1,4-dioxane, furan, tetrahydrofuran (THF), diethyl ether, ethylene glycol dimethyl ether, ethylene glycol diethyl ether, diethylene glycol dimethyl ether (diglyme), diethylene glycol diethyl ether, triethylene glycol dimethyl ether, anisole, tert-butyl methyl ether, mixtures thereof and the like.

Suitable solvents can include protic solvents (e.g., polar protic solvents) can include, by way of example and without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, iso-butyl alcohol, tert-butyl alcohol, 2-ethoxyethanol, diethylene glycol, 1-, 2-, or 3-pentanol, neo-pentyl alcohol, tert-pentyl alcohol, diethylene glycol monomethyl ether, diethylene glycol monoethyl ether, cyclohexanol, benzyl alcohol, phenol, or glycerol.

Suitable solvents can include aprotic solvents can include, by way of example and without limitation, N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide.

Suitable solvents can include hydrocarbon solvents include benzene, cyclohexane, pentane, hexane, toluene, cycloheptane, methylcyclohexane, heptane, ethylbenzene, m-, o-, or p-xylene, octane, indane, nonane, or naphthalene.

As used herein, the term “peak” or “characteristic peak” refers to a reflection having a relative height/intensity of at least about 3% of the maximum peak height/intensity.

As used herein, “pharmaceutically acceptable” or “physiologically acceptable” refer to compounds (e.g., solid forms), compositions, dosage forms and other materials, which are useful in preparing a pharmaceutical composition that is suitable for veterinary or human pharmaceutical use.

The term “pharmaceutically acceptable excipient” refers to a non-toxic binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccant, plasticizer, vehicle, disintegrant, or lubricant that does not destroy the pharmacological activity of the compound with which it is formulated. Pharmaceutically acceptable excipients that can be used in the compositions include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, “prevention” or “preventing” refers to a regimen that protects against the onset of the disorder such that the clinical symptoms of the disorder do not develop. Accordingly, “prevention” relates to administration of a therapy, including administration of a compound disclosed herein, to a subject before signs of the diseases are detectable in the subject (for example, administration of a compound disclosed herein to a subject in the absence of a detectable syndrome of the disorder). The subject may be at risk of developing the disorder. As used herein, an “at risk” subject is one who is at risk of developing a disorder to be treated. This may be shown, for example, by one or more risk factors, which are measurable parameters that correlate with development of a disorder and are known in the art.

Preparation of compounds can involve the protection and deprotection of various chemical groups. The need for protection and deprotection, and the selection of appropriate protecting groups (PG) can be readily determined by one skilled in the art. The chemistry of protecting groups can be found, for example, in Wuts and Greene, Greene's Protective Groups in Organic Synthesis, 4^{th} Ed., John Wiley & Sons: New York, 2006, which is incorporated herein by reference in its entirety. Preparation of compounds can also include leaving group (LG), which is a molecular fragment that leaves in bond cleavage. Leaving groups can be anions or neutral fragment and is able to stabilize the additional electron density that results from bond cleavage. Typical leaving groups are halides such as Cl, Br, and I, and sulfonate esters such as tosylate (TsO), triflate (TfO), mesylate (MsO), and the like.

As used herein, the term “reacting,” “contacting” or “treating” when describing a certain process is used as known in the art and generally refers to the bringing together of chemical reagents in such a manner so as to allow their interaction at the molecular level to achieve a chemical or physical transformation. In some embodiments, the reacting involves two reagents, wherein one or more equivalents of second reagent are used with respect to the first reagent. The reacting steps of the processes described herein can be conducted for a time and under conditions suitable for preparing the identified product.

As used herein, the term “salt” refers to a substance that results from the combination of a compound and an acid or a base. For example, the free base Compound 1 can be combined with the desired acid in a solvent or in a melt to generate a salt of Compound 1. In some embodiments, acid addition salt of Compound 1 can be converted to a different acid addition salt by anion exchange. Salts which are prepared in a solvent system can be isolated by precipitation from the solvent. Precipitation and/or crystallization can be induced, for example, by evaporation, reduction of temperature, addition of anti-solvent, or combinations thereof.

As used herein, the term “solid form” refers to a compound provided herein in either an amorphous state or a crystalline state (e.g., crystalline form), whereby a compound provided herein in a crystalline state may optionally include solvent or water within the crystalline lattice, for example, to form a solvated or hydrated crystalline form. In some embodiments, the compound provided herein is in a crystalline state as described herein.

A “solvate” as used herein is formed by the interaction of a solvent and a compound.

As used herein, the term “subject,” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The “subject” may have independently been diagnosed with a disorder as defined herein, may currently be experiencing symptoms associated with disorders or may have experienced symptoms in the past, may be at risk of developing a disorder, or may be reporting one or more of the symptoms of a disorder, even though a diagnosis may not have been made. In some embodiments, the subject is a human who may have independently been diagnosed with a disorder as defined herein, may currently be experiencing symptoms associated with disorders or may have experienced symptoms in the past, may be at risk of developing a disorder, or may be reporting one or more of the symptoms of a disorder, even though a diagnosis may not have been made.

As used herein, the term “substantially” when referring to a characteristic figure of a crystal form, such as an XRPD pattern, a DSC thermogram, a TGA thermogram, or the like, means that a subject figure can be non-identical to the reference depicted herein, but it falls within the limits of experimental error and thus can be deemed as derived from the same crystal form as disclosed herein, as judged by a person of ordinary skill in the art. For example, the term “substantially” as used in the context of XRPD herein is meant to encompass variations disclosed herein (e.g., instrument variation, measurement variation, etc.).

As used herein, the term “substantially amorphous” means a majority of the weight of a sample or preparation (e.g., of a salt of Compound 1) is amorphous and the remainder of the sample is a crystalline form of the same compound. In some embodiments, a substantially amorphous sample has less than about 5% crystallinity (e.g., about 95% of the non-crystalline form of the same compound), preferably less than about 4% crystallinity (e.g., about 96% of the non-crystalline form of the same compound), more preferably less than about 3% crystallinity (e.g., about 97% of the non-crystalline form of the same compound), even more preferably less than about 2% crystallinity (e.g., about 98% of the non-crystalline form of the same compound), still more preferably less than about 1% crystallinity (e.g., about 99% of the non-crystalline form of the same compound), and most preferably about 0% crystallinity (e.g., about 100% of the non-crystalline form of the same compound). In some embodiments, the term “fully amorphous” means less than about 99% or about 0% crystallinity.

As used herein, the term “substantially crystalline,” means a majority of the weight of a sample or preparation (e.g., of a salt of Compound 1) is crystalline and the remainder of the sample is a non-crystalline form (e.g., amorphous form) of the same compound. In some embodiments, a substantially crystalline sample has at least about 95% crystallinity (e.g., about 5% of the non-crystalline form of the same compound), preferably at least about 96% crystallinity (e.g., about 4% of the non-crystalline form of the same compound), more preferably at least about 97% crystallinity (e.g., about 3% of the non-crystalline form of the same compound), even more preferably at least about 98% crystallinity (e.g., about 2% of the non-crystalline form of the same compound), still more preferably at least about 99% crystallinity (e.g., about 1% of the non-crystalline form of the same compound), and most preferably about 100% crystallinity (e.g., about 0% of the non-crystalline form of the same compound). In some embodiments, the term “fully crystalline” means at least about 99% or about 100% crystallinity.

The term “substantially isolated” is meant that the compound is at least partially or substantially separated from the environment in which it was formed or detected. Partial separation can include, e.g., a composition enriched in the compound, salts, hydrates, solvates, or solid forms provided herein. Substantial separation can include compositions containing at least about 50%, at least about 60%, at least about 70%, at least about 80%, at least about 90%, at least about 95%, at least about 97%, or at least about 99% by weight of the compound, salts, hydrates, solvates, or solid forms provided herein.

As used herein, the term “therapeutically effective amount” or “effective amount” refers to an amount that is effective to elicit the desired biological or medical response, including the amount of a compound that, when administered to a subject for treating a disorder, is sufficient to effect such treatment of the disorder. The effective amount will vary depending on the compound, the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, the terms “treatment,” “treat,” and “treating” refer to an approach for obtaining beneficial or desired results including, but not limited to, therapeutic benefit. Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder. In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life). In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “treatment-resistant depression,” which is also known as “treatment-refractory depression,” refers to major depressive disorder (MDD) situations where the subject shows inadequate responses to treatment with at least two antidepressants (e.g., standard antidepressant treatments that are commercially available). Inadequate response can be no response. Inadequate response can also be when the subject does not show full remission of symptoms, or when the physician or clinician does not deem the subject's response to be adequate. Treatment-resistant depression symptoms can range from mild to severe. Factors that can contribute to inadequate response include, but not limited to, early discontinuation of treatment, insufficient dosage of medication, patient noncompliance, misdiagnosis, and concurrent psychiatric disorders.

EtOAc (ethyl acetate); g (gram(s)); h (hour(s)); HCl (hydrochloric acid); M (molar); MeCN (acetonitrile); MeOH (methanol); mg (milligram(s)); min. (minutes(s)); mL (milliliter(s)); mmol (millimole(s)); NaHCO₃ (sodium bicarbonate); NaOH (sodium hydroxide); nM (nanomolar); Ph (phenyl); μg (microgram(s)); μL (microliter(s)); μM (micromolar); wt % (weight percent).

### Salts and Crystalline Forms Thereof

Provided herein are salts of (R)-1-(8-fluoroisochroman-1-yl)-N-methylmethanamine (Compound 1), and crystalline forms thereof. Compound (R)-1-(8-fluoroisochroman-1-yl)-N-methylmethanamine (Compound 1) has the structure:

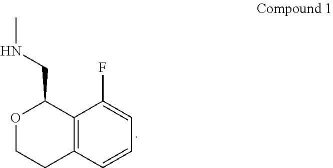

Compound 1 is described in U.S. patent application Ser. No. 15/663,688 (allowed), the entirety of which is incorporated herein by reference.

Compound 1 (R)-1-(8-fluoroisochroman-1-yl)-N-methylmethanamine is named or identified using other commonly recognized nomenclature systems. For example, the compound may be named or identified with common names, systematic names, or non-systematic names. The nomenclature systems that are commonly recognized in the art of chemistry include, but are not limited to, Chemical Abstract Service (CAS) and International Union of Pure and Applied Chemistry (IUPAC). The IUPAC name provided by ChemDraw Professional 15.0 has been used herein for Compound 1.

Compound 1 may be prepared as a salt. In some embodiments, Compound 1 may be prepared as a pharmaceutically acceptable salt. Non-limiting examples of pharmaceutically acceptable salts include malates, tartrates, citrates, phosphates, sulfates, pyrosulfates, bisulfates, sulfites, bisulfites, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, tosylates, xylenesulfonates, naphthalene-1-sulfonates, naphthalene-2-sulfonates, phenylacetates, phenylpropionates, phenylbutyrates, lactates, gamma-hydroxybutyrates, glycolates, and mandelates. Lists of other suitable pharmaceutically acceptable salts are found in Remington: The Science and Practice of Pharmacy, 21st Edition, Lippincott Williams and Wilkins, Philadelphia, Pa., 2006.

In some embodiments, the salt is a hydrochloric acid salt of Compound 1. The hydrochloric acid salt form of Compound 1 is referred to herein as “Compound 1 Hydrochloride.” An alternative name for the salt is (R)-1-(8-fluoroisochroman-1-yl)-N-methylmethanamine hydrochloride or (R)-1-(8-fluoroisochroman-1-yl)-N-methylmethanamine hydrochloric acid salt.

In some embodiments, the salt is a phosphoric acid salt of Compound 1. The phosphoric acid salt form of Compound 1 is referred to herein as “Compound 1 Phosphate.” An alternative name for the salt is (R)-1-(8-fluoroisochroman-1-yl)-N-methylmethanamine phosphate or (R)-1-(8-fluoroisochroman-1-yl)-N-methylmethanamine phosphoric acid salt.

In some embodiments, the salt is a L-tartaric acid salt of Compound 1. The L-tartaric acid salt form of Compound 1 is referred to herein as “Compound 1 L-Tartrate.” An alternative name for the salt is (R)-1-(8-fluoroisochroman-1-yl)-N-methylmethanamine or (R)-1-(8-fluoroisochroman-1-yl)-N-methylmethanamine L-tartaric acid salt.

In some embodiments, the salt is a D-tartaric acid salt of Compound 1. The D-tartaric acid salt form of Compound 1 is referred to herein as “Compound 1 D-Tartrate.” An alternative name for the salt is (R)-1-(8-fluoroisochroman-1-yl)-N-methylmethanamine D-tartrate or (R)-1-(8-fluoroisochroman-1-yl)-N-methylmethanamine D-tartaric acid salt.

In some embodiments, the salt is a fumaric acid salt of Compound 1. The fumaric acid salt form of Compound 1 is referred to herein as “Compound 1 Fumarate.” An alternative name for the salt is (R)-1-(8-fluoroisochroman-1-yl)-N-methylmethanamine fumarate or (R)-1-(8-fluoroisochroman-1-yl)-N-methylmethanamine fumaric acid salt.

In some embodiments, the salt is a citric acid salt of Compound 1. The citric acid salt form of Compound 1 is referred to herein as “Compound 1 Citrate.” An alternative name for the salt is (R)-1-(8-fluoroisochroman-1-yl)-N-methylmethanamine citrate or (R)-1-(8-fluoroisochroman-1-yl)-N-methylmethanamine citric acid salt.

In some embodiments, the salt is a succinic acid salt of Compound 1. The succinic acid salt form of Compound 1 is referred to herein as “Compound 1 Succinate.” An alternative name for the salt is (R)-1-(8-fluoroisochroman-1-yl)-N-methylmethanamine succinate or (R)-1-(8-fluoroisochroman-1-yl)-N-methylmethanamine succinic acid salt.

In some embodiments, the salt is a glutaric acid salt of Compound 1. The citric acid salt form of Compound 1 is referred to herein as “Compound 1 Glutarate.” An alternative name for the salt is (R)-1-(8-fluoroisochroman-1-yl)-N-methylmethanamine glutarate or (R)-1-(8-fluoroisochroman-1-yl)-N-methylmethanamine glutaric acid salt.

In some embodiments, the salt is a L-malic acid salt of Compound 1. The L-malic acid salt form of Compound 1 is referred to herein as “Compound 1 L-malate.” An alternative name for the salt is (R)-1-(8-fluoroisochroman-1-yl)-N-methylmethanamine L-malate or (R)-1-(8-fluoroisochroman-1-yl)-N-methylmethanamine L-malic acid salt.

In some embodiments, the salt is a benzenesulfonic acid salt of Compound 1. The benzenesulfonic acid salt form of Compound 1 is referred to herein as “Compound 1 Besylate.” An alternative name for the salt is (R)-1-(8-fluoroisochroman-1-yl)-N-methylmethanamine besylate or (R)-1-(8-fluoroisochroman-1-yl)-N-methylmethanamine benzenesulfonic acid salt.

In some embodiments, the salt is a p-toluenesulfonic acid salt of Compound 1. The p-toluenesulfonic acid salt form of Compound 1 is referred to herein as “Compound 1 Tosylate.” An alternative name for the salt is (R)-1-(8-fluoroisochroman-1-yl)-N-methylmethanamine tosylate or (R)-1-(8-fluoroisochroman-1-yl)-N-methylmethanamine p-toluenesulfonic acid salt.

The salts described herein can have about half, about 1, about 2, about 3 equivalents, etc. of acid to Compound 1. In some embodiments, the salts described herein comprises about half equivalent of acid to Compound 1. In some embodiments, the salts described herein comprise about 1 equivalent of acid to Compound 1. In some embodiments, the salts described herein comprise about 2 equivalents of acid to Compound 1. In some embodiments, the salts described herein comprise about 3 equivalents of acid to Compound 1. A person skilled in the art would recognize that there is an equilibrium between the acid and Compound 1 in which the protons may reside, which depends on the conditions (e.g., solvents, temperature, etc.) and the strength of the acids. For example, in some conditions, the acid becomes a counter-anion by losing one or more protons to Compound 1, and Compound 1 becomes a counter-cation. In some conditions, the protons of the acids may form a weak interaction with the basic sites of Compound 1 and thus, the protons are shared between the acid and Compound 1.

The salts described herein can have less than about 1, about 2, about 3, about 4, about 5, or greater than about 6 equivalents of solvent or hydrate to the salt. In some embodiments, the salts described have less than about 1 equivalent of solvent or hydrate to the salt. In some embodiments, the salts described have less than about 1 equivalent of hydrate to the salt. In some embodiments, the salts described have about 2 equivalents of solvent or hydrate to the salt. In some embodiments, the salts described have about 2 equivalent of hydrate to the salt. In some embodiments, the salts described have about 3 equivalents of solvent or hydrate to the salt. In some embodiments, the salts described have about 3 equivalents of hydrate to the salt.

In some embodiments, the salts described herein are anhydrous.

Salts of Compound 1 can be isolated as one or more crystalline forms. Different crystalline forms of the same substance may have different bulk properties relating to, for example, hygroscopicity, solubility, stability, and the like. Crystalline forms with high melting points may have good thermodynamic stability, which may be advantageous in prolonging shelf-life drug formulations containing the crystalline form. Crystalline forms with lower melting points may be less thermodynamically stable, but may be advantageous in having increased water solubility, which may translate to increased drug bioavailability. Crystalline forms that are weakly hygroscopic may be desirable for stability to heat or humidity and may be resistant to degradation during long storage. The crystalline forms described herein have many advantages, for example they have desirable properties. Moreover, the crystalline forms disclosed herein may be useful for improving the performance characteristics of a pharmaceutical product such as dissolution profile, shelf-life and bioavailability.

Different crystalline forms of a particular substance, such as Compound 1 as described herein, can include both anhydrous forms of that substance and solvated/hydrated forms of that substance, where each of the anhydrous forms and solvated/hydrated forms are distinguished from each other by different XRPD patterns, or other solid state characterization methods, thereby signifying different crystalline lattices. In some instances, a single crystalline form (e.g., identified by a unique XRPD pattern) can have variable water or solvent content, where the lattice remains substantially unchanged (as does the XRPD pattern) despite the compositional variation with respect to water and/or solvent.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, filters used, the sample mounting procedure, and the particular instrument employed. In some instances, new peaks can be observed or existing peaks may disappear, depending on the type of the machine or the settings (for example, whether a Ni filter is used or not). Moreover, instrument variation and other factors can affect the 2-theta (2θ) values. Thus, peak assignments, such as those reported herein, can vary by plus or minus (±) about 0.2° (2-theta) or about 0.3° (2-theta).

In the same way, temperature readings in connection with DSC, TGA, or other thermal experiments can vary about ±3° C. depending on the instrument, particular settings, sample preparation, etc. Accordingly, a crystalline form reported herein having a DSC thermogram “substantially” as shown in any of the Figures is understood to accommodate such variation.

Crystalline forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates such as, e.g., on polymers, recrystallization in the presence of additives, such as, e.g., co-crystal counter-molecules, desolvation, dehydration, rapid evaporation, rapid cooling, slow cooling, vapor diffusion, sublimation, exposure to moisture, grinding and solvent-drop grinding.

Compound 1 and its salts can be prepared in batches referred to as batches, samples, or preparations. The batches, samples, or preparations can include Compound 1 and its salts in any of the crystalline or non-crystalline forms described herein, including hydrated and non-hydrated forms, and mixtures thereof.

Compounds provided herein (e.g., salts of Compound 1) can also include all isotopes of atoms occurring in the intermediates or final compounds. Isotopes include those atoms having the same atomic number but different mass numbers. For example, isotopes of hydrogen include tritium and deuterium. One or more constituent atoms of the compounds provided herein can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7 or 8 deuterium atoms. Synthetic methods for including isotopes into organic compounds are known in the art. Examples of isotopes that can be incorporated into the compounds disclosed herein include isotopes of hydrogen, carbon, nitrogen, oxygen, phosphorus, fluorine, chlorine, and iodine (e.g., ²H ³H, ¹¹C, ¹³C, ¹⁴C, ¹³N, ¹⁵N, ¹⁵O, ¹⁸O, ³¹P, ³²P, ³⁵S, ¹⁸F, ³⁶Cl, ¹²³I and ¹²⁵I).

In some embodiments, Compound 1 or its salts and crystalline forms thereof are substantially isolated.

Compound 1 can be observed and/or isolated as various salt forms and polymorphs thereof, including, e.g., hydrochloride salt (form HA and form HB), phosphate salt, L-tartrate salt (form LA, form LB, and form LC), D-tartrate salt, fumarate (form FA and form FB), citrate, succinate, glutarate, L-malate, besylate, and tosylate.

### Compound 1 Hydrochloride

In some embodiments, provided is (R)-1-(8-fluoroisochroman-1-yl)-N-methylmethanamine hydrochloride (Compound 1 Hydrochloride). In some embodiments, Compound 1 Hydrochloride is crystalline.

Compound 1 Hydrochloride can be prepared according to the procedure provided in U.S. Pat. No. 10,196,403. In some embodiments, provided is Compound 1 Hydrochloride prepared by isolating Compound 1 Hydrochloride Form HA from a mixture of Compound 1, HCl, and S1, wherein S1 is a solvent. In some embodiments, S1 is an organic solvent. In some embodiments, S1 is C₁₋₆ alkyl alcohol. In some embodiments, S1 is ether. In some embodiments, S1 is C₁₋₆ alkyl acetate. In some embodiments, S1 is methanol. In some embodiments, S1 is THF. In some embodiments, S1 is ethyl acetate.

### Compound 1 Hydrochloride Form HA

In some embodiments, provided is (R)-1-(8-fluoroisochroman-1-yl)-N-methylmethanamine hydrochloride Form HA (Compound 1 hydrochloride Form HA). In some embodiments, Compound 1 Hydrochloride Form HA is crystalline.

In some embodiments, Compound 1 Hydrochloride Form HA has characteristic XRPD peak in terms of 2θ selected from 9.4°±0.2°, 11.4±0.2°, and 15.1°±0.2°. In some embodiments, Compound 1 Hydrochloride Form HA has a characteristic XRPD peak in terms of 2θ at 9.4°±0.2°. In some embodiments, Compound 1 Hydrochloride Form HA has a characteristic XRPD peak in terms of 2θ at 11.40±0.2°. In some embodiments, Compound 1 Hydrochloride Form HA has a characteristic XRPD peak in terms of 2θ at 15.10±0.2°.

In some embodiments, Compound 1 Hydrochloride Form HA has characteristic XRPD peaks in terms of 2θ selected from 9.4°±0.2°, 11.4±0.2°, 15.1°±0.2°, 17.2°±0.2°, and 17.6°±0.2°. In some embodiments, Compound 1 Hydrochloride Form HA has at least one characteristic XRPD peak in terms of 2θ selected from 9.4°±0.2°, 11.4±0.2°, 15.10°±0.2°, 17.2°±0.2°, and 17.6°±0.2°.

In some embodiments, Compound 1 Hydrochloride Form HA has at least one characteristic XRPD peak in terms of 2θ selected from 9.4°±0.2°, 11.4°±0.2°, 14.2°±0.2°, 15.1°±0.2°, 17.2°±0.2°, 17.6°±0.2°, and 27.0°±0.2°. In some embodiments, Compound 1 Hydrochloride Form HA has at least one characteristic XRPD peak in terms of 2θ selected from 9.4°±0.2°, 11.4°±0.2°, 14.2°±0.2°, 15.1°±0.2°, 17.2°±0.2°, 17.6°±0.2°, 18.8°±0.2°, 19.2°±0.20, 24.3°±0.20, and 27.0°±0.20.

In some embodiments, Compound 1 Hydrochloride Form HA has at least two characteristic XRPD peaks in terms of 2θ selected from 9.4°±0.2°, 11.4°±0.2°, 14.2°±0.2°, 15.1°±0.2°, 17.2°±0.2°, 17.6°±0.2°, and 27.0°±0.2°. In some embodiments, Compound 1 Hydrochloride Form HA has at least two characteristic XRPD peaks in terms of 2θ selected from 9.4°±0.2°, 11.4°±0.2°, 14.2°±0.2°, 15.1°±0.2°, 17.2°±0.2°, 17.6°±0.2°, 18.8°±0.2°, 19.2°±0.20, 24.3°±0.20, and 27.0°±0.20.

In some embodiments, Compound 1 Hydrochloride Form HA has at least three characteristic XRPD peaks in terms of 2θ selected from 9.4°±0.2°, 11.4°±0.2°, 14.2°±0.2°, 15.1°±0.2°, 17.2°±0.2°, 17.6°±0.2°, and 27.0°±0.2°. In some embodiments, Compound 1 Hydrochloride Form HA has at least three characteristic XRPD peaks in terms of 2θ selected from 9.4°±0.2°, 11.4°±0.2°, 14.2°±0.2°, 15.1°±0.2°, 17.2°±0.2°, 17.6°±0.2°, 18.8°±0.2°, 19.2°±0.2°, 24.3°±0.2°, and 27.0°±0.2°.

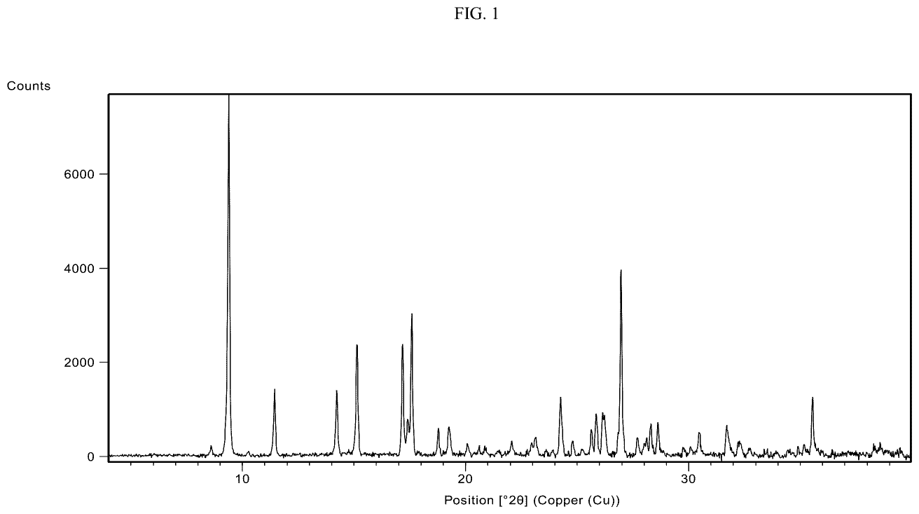

In some embodiments, Compound 1 Hydrochloride Form HA has an XRPD pattern with characteristic peaks as substantially shown in FIG. 1 (FIG. 1).

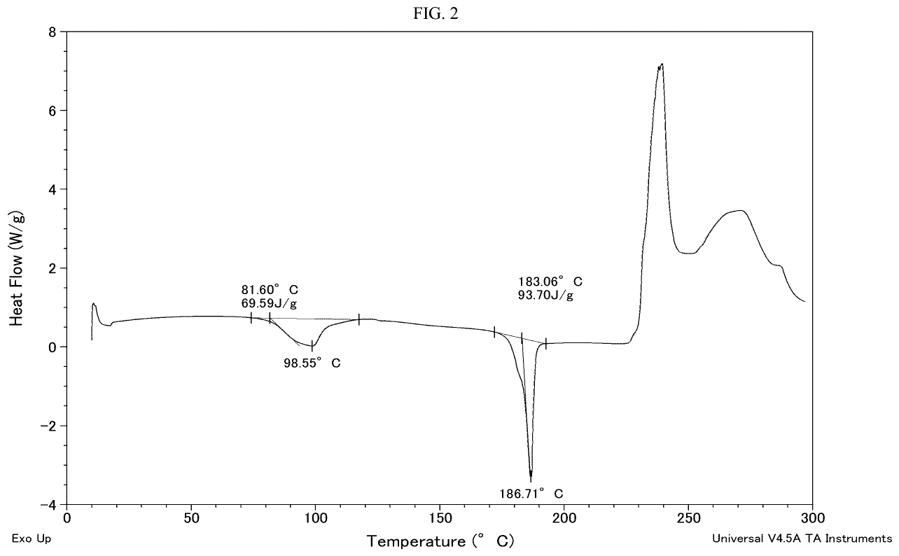

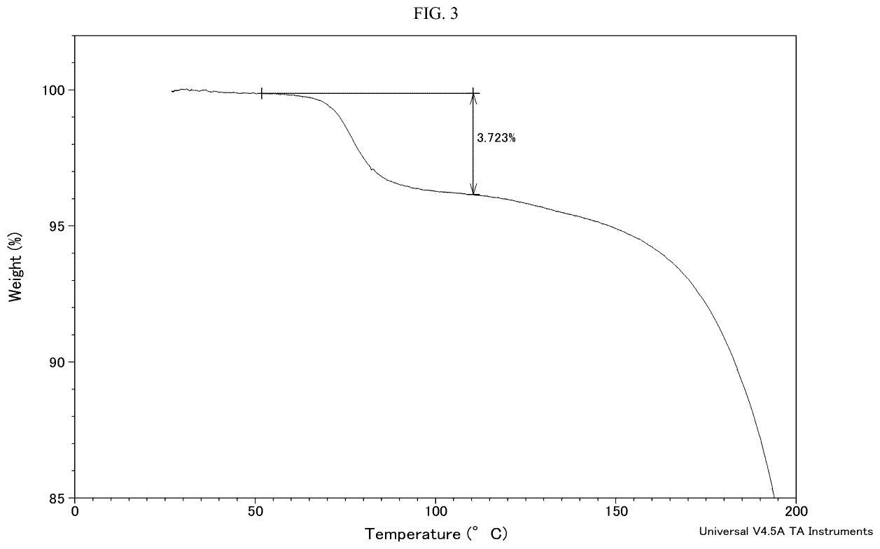

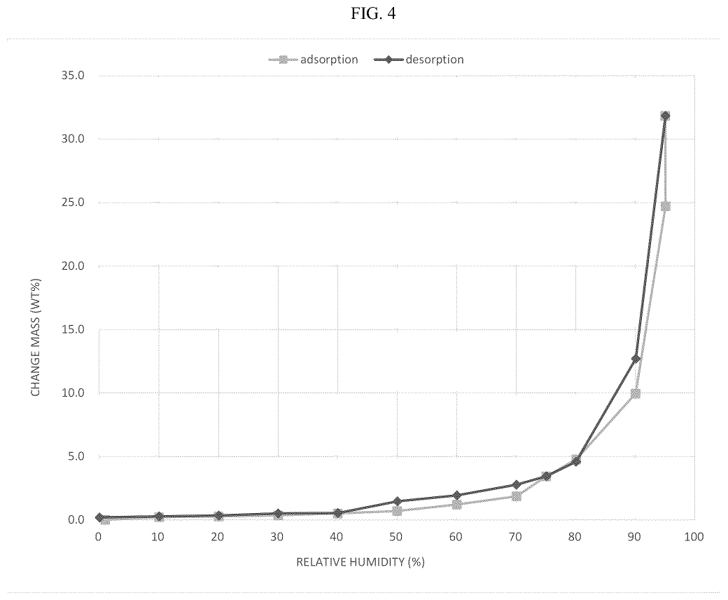

In some embodiments, Compound 1 Hydrochloride Form HA has endotherm peaks at temperatures of about 99° C. and about 187° C. In some embodiments, Compound 1 Hydrochloride Form HA has an endotherm peak at a temperature of about 99° C. In some embodiments, Compound 1 Hydrochloride Form HA has an endotherm peak at a temperature of about 187° C. In some embodiments, Compound 1 Hydrochloride Form HA has a DSC thermogram substantially as depicted in FIG. 2 (FIG. 2). In some embodiments, Compound 1 Hydrochloride Form HA has a TGA thermogram substantially as depicted in FIG. 3 (FIG. 3). In some embodiments, Compound 1 Hydrochloride Form HA has a DVS isotherm substantially as depicted in FIG. 4 (FIG. 4).

In some embodiments, Compound 1 Hydrochloride Form HA has characteristic XRPD peaks in terms of 2θ selected from 9.4°±0.2°, 11.4±0.2°, and 15.1°±0.2°; and has endotherm peaks at temperatures of about 99° C. and about 187° C. In some embodiments, Compound 1 Hydrochloride Form HA has characteristic XRPD peaks in terms of 2θ selected from 9.4°±0.2°, 11.4±0.2°, and 15.1°±0.2°; and an endotherm peak at a temperature of about 99° C. In some embodiments, Compound 1 Hydrochloride Form HA has characteristic XRPD peaks in terms of 2θ selected from 9.4°±0.2°, 11.4±0.2°, and 15.1°±0.2°; and an endotherm peak at a temperature of about 187° C. In some embodiments, Compound 1 Hydrochloride Form HA has characteristic XRPD peaks in terms of 2θ selected from 9.4°±0.2°, 11.4±0.2°, and 15.10°±0.2°; and a DSC thermogram substantially as depicted in FIG. 2 (FIG. 2). In some embodiments, Compound 1 Hydrochloride Form HA has characteristic XRPD peaks in terms of 2θ selected from 9.4°±0.2°, 11.4±0.2°, and 15.10°±0.2°; and a DVS isotherm substantially as depicted in FIG. 4 (FIG. 4).

In some embodiments, Compound 1 Hydrochloride Form HA can be isolated with a crystalline purity of at least about 80%, about 85%, about 90%, about 95%, about 96%, about 97%, about 98%, or about 99%. In some embodiments, Compound 1 Hydrochloride Form HA can be isolated with a crystalline purity greater than about 99%. In some embodiments, Compound 1 Hydrochloride Form HA can be isolated with a crystalline purity greater than about 99.9%.

In some embodiments, provided is Compound 1 Hydrochloride Form HA prepared by isolating Compound 1 Hydrochloride Form HA from a mixture of Compound 1, HCl, and S1, wherein S1 is a solvent. In some embodiments, S1 is an organic solvent. In some embodiments, S1 is ether. In some embodiments, S1 is C₁₋₆ alkyl acetate. In some embodiments, S1 is THF. In some embodiments, S1 is ethyl acetate.

### Compound 1 Hydrochloride Form HB

In some embodiments, provided is (R)-1-(8-fluoroisochroman-1-yl)-N-methylmethanamine Hydrochloride Form HB (Compound 1 hydrochloride Form HB). In some embodiments, Compound 1 Hydrochloride HB is crystalline.

In some embodiments, Compound 1 Hydrochloride Form HB has characteristic XRPD peaks in terms of 2θ selected from 8.6°±0.2°, 9.6°±0.2°, and 10.3°±0.2°. In some embodiments, Compound 1 Hydrochloride Form HB has a characteristic XRPD peak in terms of 2θ at 8.6°±0.2°. In some embodiments, Compound 1 Hydrochloride Form HB has a characteristic XRPD peak in terms of 2θ at 9.6°±0.2°. In some embodiments, Compound 1 Hydrochloride HB has a characteristic XRPD peak in terms of 2θ at 10.3°±0.2°.

In some embodiments, Compound 1 Hydrochloride Form HB has characteristic XRPD peaks in terms of 2θ selected from 8.6°±0.2°, 9.6°±0.2°, 10.3°±0.2°, and 17.3°±0.2°. In some embodiments, Compound 1 Hydrochloride Form HB has at least one characteristic XRPD peak in terms of 2θ selected from 8.6°±0.2°, 9.6°±0.2°, 10.3°±0.2°, and 17.3°±0.2°.

In some embodiments, Compound 1 Hydrochloride Form HB has at least one characteristic XRPD peak in terms of 2θ selected from 8.6°±0.2°, 9.6°±0.2°, 10.3°±0.2°, 12.6°±0.2°, 14.7°±0.2°, 17.3°±0.2°, and 23.8°±0.2°. In some embodiments, Compound 1 Hydrochloride Form HB has at least one characteristic XRPD peak in terms of 2θ selected from 8.6°±0.2°, 9.6°±0.2°, 10.3°±0.2°, 12.6°±0.2°, 14.7°±0.2°, 16.5°±0.2°, 17.3°±0.2°, 18.3°±0.20, 23.8°±0.20, 24.4°±0.20, 26.9°±0.20, and 27.1°±0.20.

In some embodiments, Compound 1 Hydrochloride Form HB has at least two characteristic XRPD peaks in terms of 2θ selected from 8.6°±0.2°, 9.6°±0.2°, 10.3°±0.2°, 12.6°±0.2°, 14.7°±0.2°, 17.3°±0.2°, and 23.8°±0.2°. In some embodiments, Compound 1 Hydrochloride Form HB has at least two characteristic XRPD peaks in terms of 2θ selected from 8.6°±0.2°, 9.6°±0.2°, 10.3°±0.2°, 12.6°±0.2°, 14.7°±0.2°, 16.5°±0.2°, 17.3°±0.2°, 18.3°±0.20, 23.8°±0.20, 24.4°±0.20, 26.9°±0.20, and 27.1°±0.20.

In some embodiments, Compound 1 Hydrochloride Form HB has at least three characteristic XRPD peaks in terms of 2θ selected from 8.6°±0.2°, 9.6°±0.2°, 10.3°±0.2°, 12.6°±0.2°, 14.7°±0.2°, 17.3°±0.2°, and 23.8°±0.2°. In some embodiments, Compound 1 Hydrochloride Form HB has at least three characteristic XRPD peaks in terms of 2θ selected from 8.60±0.20, 9.60±0.20, 10.3°±0.20, 12.6°±0.20, 14.7°±0.20, 16.5°±0.20, 17.3°±0.20, 18.3°±0.2°, 23.8°±0.2°, 24.4°±0.2°, 26.9°±0.2°, and 27.1°±0.2°.

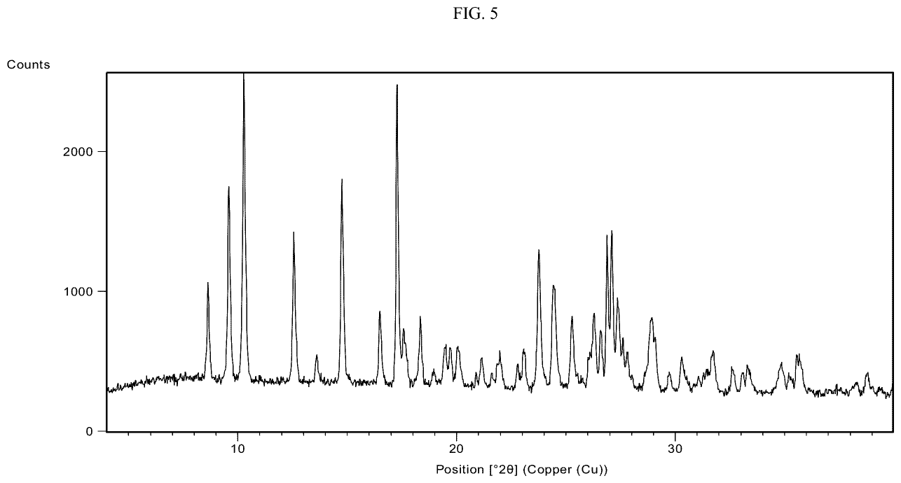

In some embodiments, Compound 1 Hydrochloride Form HB has an XRPD pattern with characteristic peaks as substantially shown in FIG. 5 (FIG. 5).

In some embodiments, Compound 1 Hydrochloride Form HB can be isolated with a crystalline purity of at least about 80%, about 85%, about 90%, about 95%, about 96%, about 97%, about 98%, or about 99%. In some embodiments, Compound 1 Hydrochloride Form HB can be isolated with a crystalline purity greater than about 99%. In some embodiments, Compound 1 Hydrochloride Form HB can be isolated with a crystalline purity greater than about 99.9%.

In some embodiments, provided is Compound 1 hydrochloride Form HB prepared by isolating Compound 1 hydrochloride Form HA from a mixture of Compound 1, HCl, and S1, wherein S1 is a solvent. In some embodiments, S1 comprises water. In some embodiments, Form HB is prepared by exposing Form HA to high humidity. In some embodiments, Form HB is prepared by exposing Form HA to about 75% relative humidity.

### Compound 1 Phosphate

In some embodiments, provided is (R)-1-(8-fluoroisochroman-1-yl)-N-methylmethanamine Phosphate. In some embodiments, Compound 1 Phosphate is crystalline.

In some embodiments, Compound 1 Phosphate has characteristic XRPD peaks in terms of 2θ selected from 4.6°±0.2°, 9.1°±0.2°, and 18.2°±0.2°. In some embodiments, Compound 1 Phosphate has a characteristic XRPD peak in terms of 2θ at 4.6°±0.2°. In some embodiments, Compound 1 Phosphate has a characteristic XRPD peak in terms of 2θ at 9.1°±0.2°. In some embodiments, Compound 1 Phosphate has a characteristic XRPD peak in terms of 2θ at 18.2°±0.2°.

In some embodiments, Compound 1 Phosphate has characteristic XRPD peaks in terms of 2θ selected from 4.6°±0.2°, 9.1°±0.2°, 18.2°±0.2°, and 22.8°±0.2°. In some embodiments, Compound 1 Phosphate has at least one characteristic XRPD peak in terms of 2θ selected from 4.6°±0.2°, 9.1°±0.2°, 18.2°±0.2°, and 22.8°±0.2°.

In some embodiments, Compound 1 Phosphate has at least one characteristic XRPD peak in terms of 2θ selected from 4.6°±0.2°, 9.1°±0.2°, 15.7°±0.2°, 18.2°±0.2°, 22.3°±0.2°, 22.8°±0.2°, and 24.8°±0.2°. In some embodiments, Compound 1 Phosphate has at least one characteristic XRPD peak in terms of 2θ selected from 4.6°±0.2°, 9.1°±0.2°, 15.7°±0.2°, 18.2°±0.2°, 19.1°±0.2°, 22.3°±0.2°, 22.8°±0.2°, 24.8°±0.2°, 26.0°±0.2°, 27.4°±0.2°, and 30.1°±0.2°.

In some embodiments, Compound 1 Phosphate has at least two characteristic XRPD peaks in terms of 2θ selected from 4.6°±0.2°, 9.1°±0.2°, 15.7°±0.2°, 18.2°±0.2°, 22.3°±0.2°, 22.8°±0.2°, and 24.8°±0.2°. In some embodiments, Compound 1 Phosphate has at least two characteristic XRPD peaks in terms of 2θ selected from 4.6°±0.2°, 9.1°±0.2°, 15.7°±0.2°, 18.2°±0.2°, 19.1°±0.2°, 22.3°±0.2°, 22.8°±0.2°, 24.8°±0.2°, 26.0°±0.2°, 27.4°±0.2°, and 30.1°±0.2°.

In some embodiments, Compound 1 Phosphate has at least three characteristic XRPD peaks in terms of 2θ selected from 4.6°±0.2°, 9.1°±0.2°, 15.7°±0.2°, 18.2°±0.2°, 22.3°±0.2°, 22.8°±0.2°, and 24.8°±0.2°. In some embodiments, Compound 1 Phosphate has at least three characteristic XRPD peaks in terms of 2θ selected from 4.6°±0.2°, 9.1°±0.2°, 15.7°±0.2°, 18.2°±0.2°, 19.1°±0.2°, 22.3°±0.2°, 22.8°±0.2°, 24.8°±0.2°, 26.0°±0.2°, 27.4°±0.2°, and 30.1°±0.20.

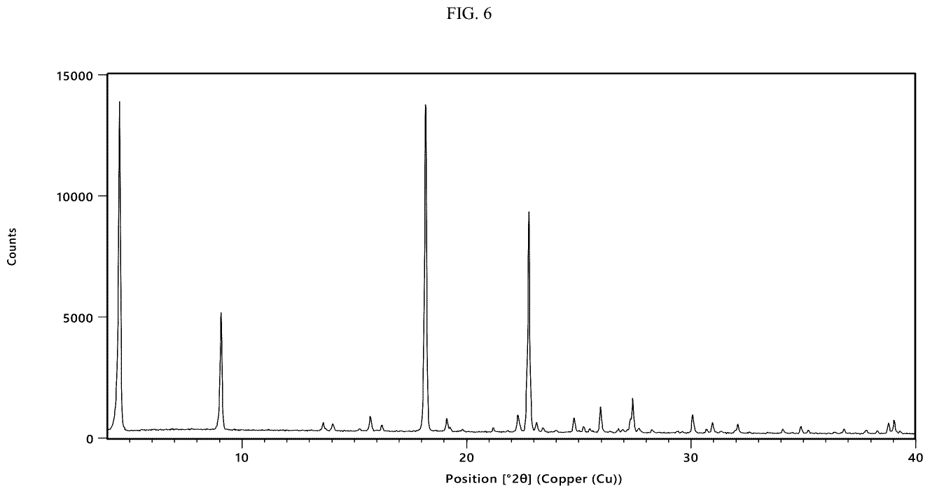

In some embodiments, Compound 1 Phosphate has an XRPD pattern with characteristic peaks as substantially shown in FIG. 6 (FIG. 6).

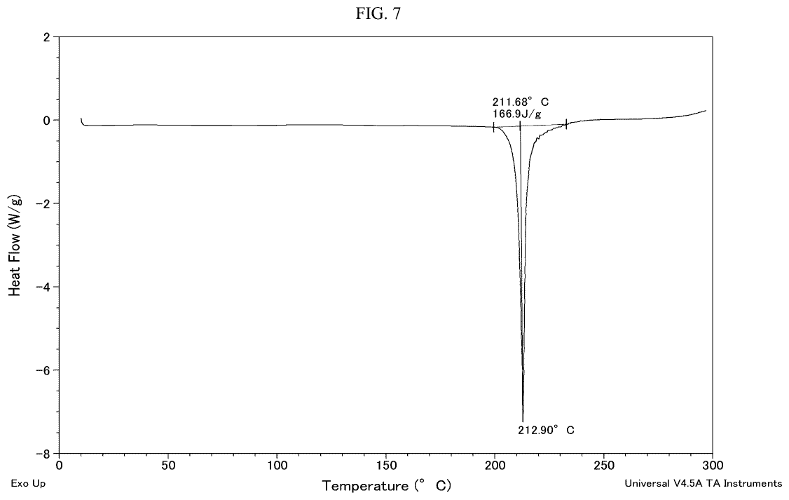

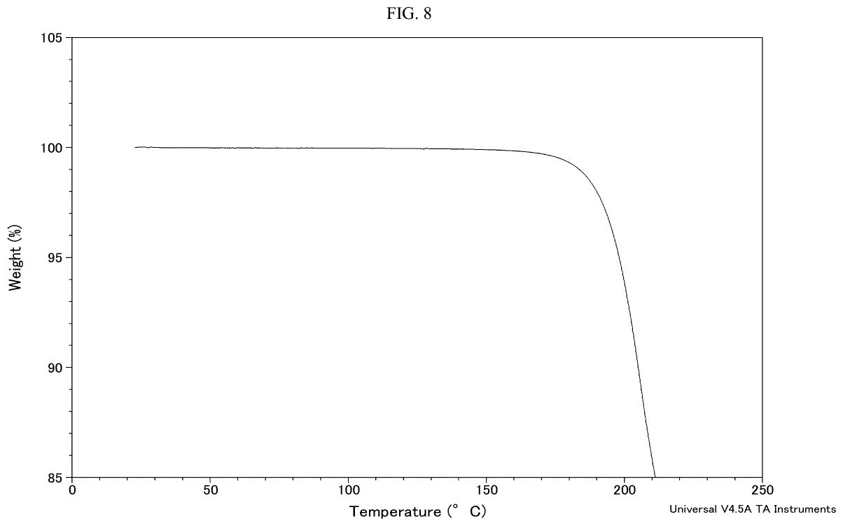

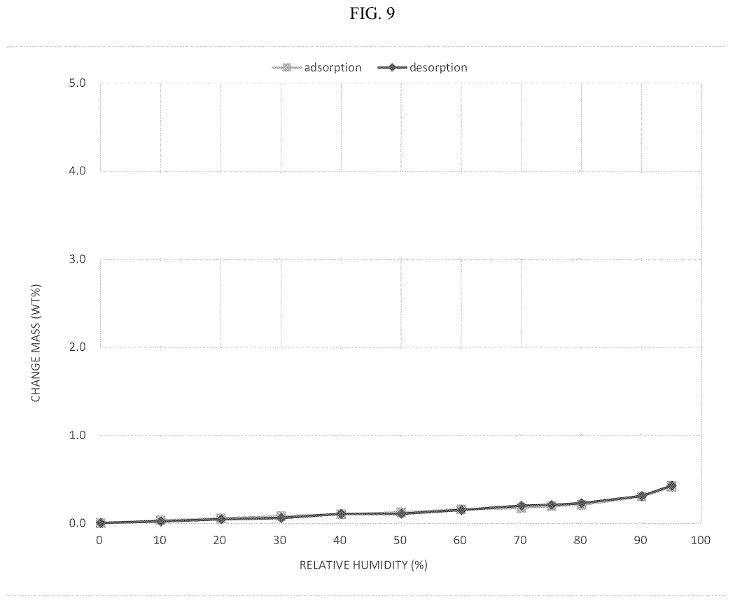

In some embodiments, Compound 1 Phosphate has an endotherm peak at a temperature of about 213° C. In some embodiments, Compound 1 Phosphate has a DSC thermogram substantially as depicted in FIG. 7 (FIG. 7). In some embodiments, Compound 1 Phosphate has a TGA thermogram substantially as depicted in FIG. 8 (FIG. 8). In some embodiments, Compound 1 Phosphate has a DVS isotherm substantially as depicted in FIG. 9 (FIG. 9).

In some embodiments, Compound 1 Phosphate has at least one characteristic XRPD peak in terms of 2θ selected from 4.6°±0.2°, 9.1°±0.2°, 18.2°±0.2°, and 22.8°±0.2°; and an endotherm peak at a temperature of about 213° C. In some embodiments, Compound 1 Phosphate has at least one characteristic XRPD peak in terms of 2θ selected from 4.6°±0.2°, 9.1°±0.2°, 18.2°±0.2°, and 22.8°±0.2°; and a DSC thermogram substantially as depicted in FIG. 7 (FIG. 7). In some embodiments, Compound 1 Phosphate has at least one characteristic XRPD peak in terms of 2θ selected from 4.6°±0.2°, 9.1°±0.2°, 18.2°±0.2°, and 22.8°±0.2°; and a DVS isotherm substantially as depicted in FIG. 9 (FIG. 9).

In some embodiments, Compound 1 Phosphate can be isolated with a crystalline purity of at least about 80%, about 85%, about 90%, about 95%, about 96%, about 97%, about 98%, or about 99%. In some embodiments, Compound 1 Phosphate can be isolated with a crystalline purity greater than about 99%. In some embodiments, Compound 1 phosphate can be isolated with a crystalline purity greater than about 99.9%.

In some embodiments, provided is Compound 1 phosphate prepared by isolating Compound 1 phosphate from a mixture of Compound 1, phosphoric acid, and S1, wherein S1 is a solvent. In some embodiments, S1 is an organic solvent. In some embodiments, S1 is C₁₋₆ alkyl alcohol. In some embodiments, S1 is ether. In some embodiments, S1 is C₁₋₆ alkyl acetate. In some embodiments, S1 is a mixture of organic solvents. In some embodiments, S1 is a mixture of C₁₋₆ alkyl alcohol and C₁₋₆ alkyl acetate. In some embodiments, S1 is THF. In some embodiments, S1 is ethyl acetate. In some embodiments, S1 is a mixture of methanol and acetone. In some embodiments, S1 is a mixture of methanol and ethyl acetate.

### Compound 1 L-Tartrate

In some embodiments, provided is (R)-1-(8-fluoroisochroman-1-yl)-N-methylmethanamine L-tartrate (Compound 1 L-Tartrate). In some embodiments, Compound 1 L-Tartrate is crystalline.

In some embodiments, provided is Compound 1 L-Tartrate prepared by isolating Compound 1 L-Tartrate from a mixture of Compound 1, tartaric acid, and S1, wherein S1 is a solvent. In some embodiments, S1 is an organic solvent. In some embodiments, S1 is C₁₋₆ alkyl alcohol. In some embodiments, S1 is ether. In some embodiments, S1 is C₁₋₆ alkyl acetate. In some embodiments, S1 is C₁₋₆ alkyl ketone. In some embodiments, S1 is a mixture of C₁₋₆ alkyl alcohol and C₁₋₆ alkyl ketone. In some embodiments, S1 is a mixture of C₁₋₆ alkyl alcohol and C₁₋₆ alkyl acetate. In some embodiments, S1 is methanol. In some embodiments, S1 is THF. In some embodiments, S1 is ethyl acetate. In some embodiments, S1 is a mixture of methanol and acetone. In some embodiments, S1 is a mixture of methanol and ethyl acetate.

### Compound 1 L-Tartrate Form LA

In some embodiments, provided is (R)-1-(8-fluoroisochroman-1-yl)-N-methylmethanamine L-Tartrate Form LA (Compound 1 L-Tartrate Form LA). In some embodiments, Compound 1 L-Tartrate Form LA is crystalline.

In some embodiments, Compound 1 L-Tartrate Form LA has characteristic XRPD peaks in terms of 2θ selected from 12.1°±0.2°, 18.1°±0.2°, and 24.2°±0.2°. In some embodiments, Compound 1 L-Tartrate Form LA has a characteristic XRPD peak in terms of 2θ at 12.1°±0.2°. In some embodiments, Compound 1 L-Tartrate Form LA has a characteristic XRPD peak in terms of 2θ at 18.10°±0.2°. In some embodiments, Compound 1 L-Tartrate Form LA has a characteristic XRPD peak in terms of 2θ at 24.2°±0.2°.

In some embodiments, Compound 1 L-Tartrate Form LA has at least one characteristic XRPD peak in terms of 2θ selected from 12.1°±0.2°, 15.0°±0.2°, 16.4°±0.2°, 16.9°±0.2°, 17.1°±0.2°, 18.1°±0.2°, 23.9°±0.2°, and 24.2°±0.2°. In some embodiments, Compound 1 L-Tartrate Form LA has at least one characteristic XRPD peak in terms of 2θ selected from 12.1°±0.2°, 15.0°±0.2°, 16.4°±0.2°, 16.9°±0.2°, 17.1°±0.2°, 18.1°±0.2°, 19.3°±0.20, 23.9°±0.20, 24.2°±0.20, 24.8°±0.20, and 27.2°±0.20.

In some embodiments, Compound 1 L-Tartrate Form LA has at least two characteristic XRPD peaks in terms of 2θ selected from 12.1°±0.2°, 15.0°±0.2°, 16.4°±0.2°, 16.9°±0.2°, 17.1°±0.2°, 18.1°±0.2°, 23.9°±0.2°, and 24.2°±0.2°. In some embodiments, Compound 1 L-Tartrate Form LA has at least two characteristic XRPD peaks in terms of 2θ selected from 12.1°±0.2°, 15.0°±0.2°, 16.4°±0.2°, 16.9°±0.2°, 17.1°±0.2°, 18.1°±0.2°, 19.3°±0.20, 23.9°±0.20, 24.2°±0.20, 24.8°±0.20, and 27.2°±0.20.

In some embodiments, Compound 1 L-Tartrate Form LA has at least three characteristic XRPD peaks in terms of 2θ selected from 12.1°±0.2°, 15.0°±0.2°, 16.4°±0.2°, 16.9°±0.2°, 17.1°±0.2°, 18.1°±0.2°, 23.9°±0.2°, and 24.2°±0.2°. In some embodiments, Compound 1 L-Tartrate Form LA has at least three characteristic XRPD peaks in terms of 2θ selected from 12.1°±0.2°, 15.0°±0.2°, 16.4°±0.2°, 16.9°±0.2°, 17.1°±0.2°, 18.1°±0.2°, 19.3°±0.20, 23.9°±0.20, 24.2°±0.20, 24.8°±0.20, and 27.2°±0.20.

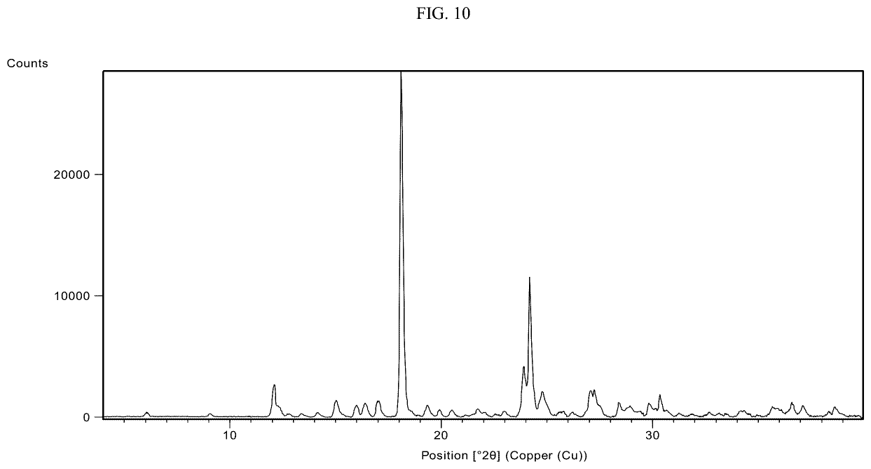

In some embodiments, Compound 1 L-Tartrate Form LA has an XRPD pattern with characteristic peaks as substantially shown in FIG. 10 (FIG. 10).

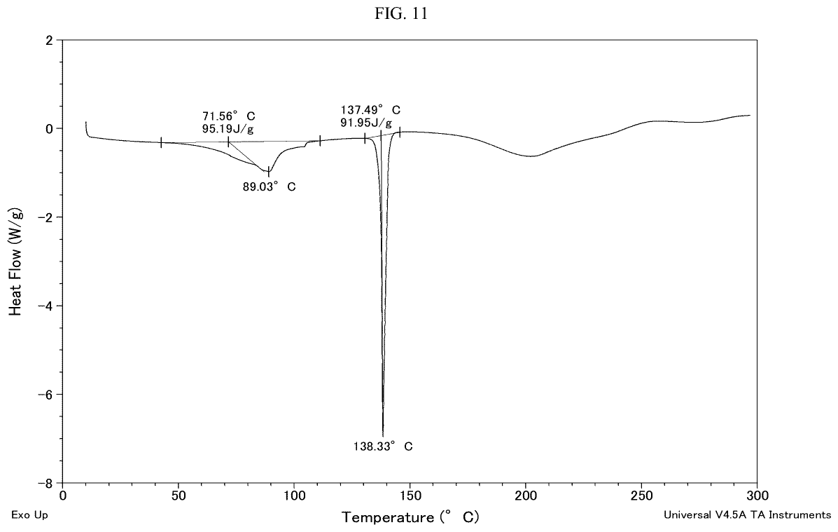

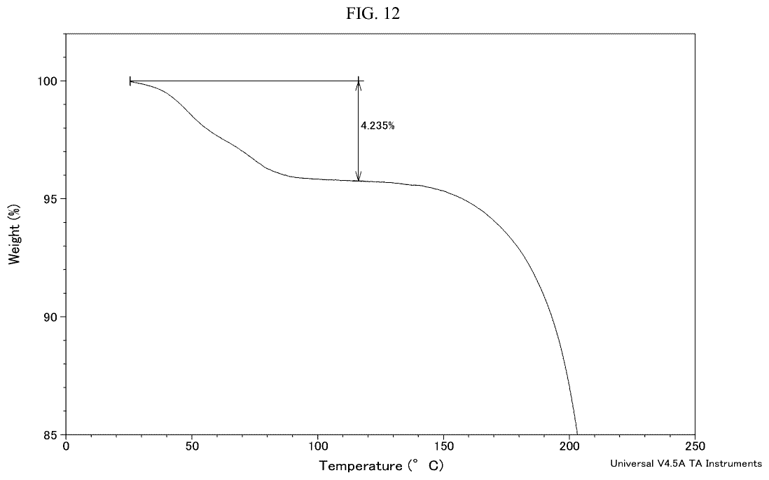

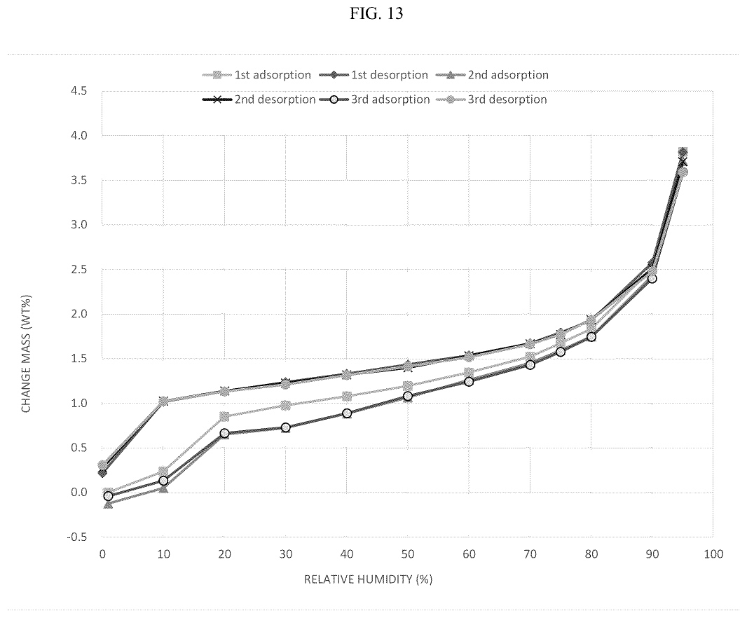

In some embodiments, Compound 1 L-Tartrate Form LA has endotherm peaks at temperatures of about 89° C. and about 138° C. In some embodiments, Compound 1 L-Tartrate Form LA has an endotherm peak at a temperature of about 89° C. In some embodiments, Compound 1 L-Tartrate Form LA has an endotherm peak at a temperature of about 138° C. In some embodiments, Compound 1 L-Tartrate Form LA has a DSC thermogram substantially as depicted in FIG. 11 (FIG. 11). In some embodiments, Compound 1 L-Tartrate Form LA has a TGA thermogram substantially as depicted in FIG. 12 (FIG. 12). In some embodiments, Compound 1 L-Tartrate Form LA has a DVS isotherm substantially as depicted in FIG. 13 (FIG. 13).

In some embodiments, Compound 1 L-Tartrate Form LA has at least one characteristic XRPD peak in terms of 2θ selected from 12.1°±0.2°, 18.1°±0.2°, and 24.2°±0.2°; and has endotherm peaks at temperatures of about 89° C. and about 138° C. In some embodiments, Compound 1 L-Tartrate Form LA has at least one characteristic XRPD peak in terms of 2θ selected from 12.1°±0.2°, 18.1°±0.2°, and 24.2°±0.2°; and an endotherm peak at a temperature of about 89° C. In some embodiments, Compound 1 L-Tartrate Form LA has at least one characteristic XRPD peak in terms of 2θ selected from 12.1°±0.2°, 18.1°±0.2°, and 24.2°±0.2°; and an endotherm peak at a temperature of about 138° C. In some embodiments, Compound 1 L-Tartrate Form LA has at least one characteristic XRPD peak in terms of 2θ selected from 12.1°±0.2°, 18.1°±0.2°, and 24.2°±0.2°; and a DSC thermogram substantially as depicted in FIG. 11 (FIG. 11). In some embodiments, Compound 1 L-Tartrate Form LA has at least one characteristic XRPD peak in terms of 2θ selected from 12.1°±0.2°, 18.1°±0.2°, and 24.2°±0.2°; and a DVS isotherm substantially as depicted in FIG. 13 (FIG. 13).

In some embodiments, Compound 1 L-Tartrate Form LA can be isolated with a crystalline purity of at least about 80%, about 85%, about 90%, about 95%, about 96%, about 97%, about 98%, or about 99%. In some embodiments, Compound 1 L-Tartrate Form LA can be isolated with a crystalline purity greater than about 99%. In some embodiments, Compound 1 L-Tartrate Form LA can be isolated with a crystalline purity greater than about 99.9%.

In some embodiments, provided is Compound 1 L-tartrate Form LA prepared by isolating Compound 1 L-tartrate Form LA from a mixture of Compound 1, L-tartaric acid, and S1, wherein S1 is a solvent. In some embodiments, S1 is an organic solvent. In some embodiments, S1 is ether. In some embodiments, S1 is C₁₋₆ alkyl acetate. In some embodiments, S1 is THF. In some embodiments, S1 is ethyl acetate.

### Compound 1 L-Tartrate Form LB

In some embodiments, provided is (R)-1-(8-fluoroisochroman-1-yl)-N-methylmethanamine L-Tartrate Form LB (Compound 1 L-tartrate Form LB). In some embodiments, Compound 1 L-Tartrate Form LB is crystalline.

In some embodiments, Compound 1 L-Tartrate Form LB has characteristic XRPD peaks in terms of 2θ selected from 18.7°±0.2°, 25.0°±0.2°, and 31.4°±0.2°. In some embodiments, Compound 1 L-Tartrate Form LB has a characteristic XRPD peak in terms of 2θ at 18.7°±0.2°. In some embodiments, Compound 1 L-Tartrate Form LB has a characteristic XRPD peak in terms of 2θ at 25.0°±0.2°. In some embodiments, Compound 1 L-Tartrate Form LB has a characteristic XRPD peak in terms of 2θ at 31.4°±0.2°.

In some embodiments, Compound 1 L-Tartrate Form LB has at least one characteristic XRPD peak in terms of 2θ selected from 6.3°±0.2°, 12.5°±0.2°, 18.1°±0.2°, 18.7°±0.2°, 23.9°±0.2°, 25.0°±0.2°, and 31.4°±0.2°. In some embodiments, Compound 1 L-Tartrate Form LB has at least one characteristic XRPD peak in terms of 2θ selected from 6.3°±0.2°, 12.5°±0.2°, 18.1°±0.2°, 18.7°±0.2°, 20.0°±0.2°, 23.9°±0.2°, 24.2°±0.2°, 25.0°±0.2°, and 31.4°±0.20.

In some embodiments, Compound 1 L-Tartrate Form LB has at least two characteristic XRPD peaks in terms of 2θ selected from 6.3°±0.2°, 12.5°±0.2°, 18.1°±0.2°, 18.7°±0.2°, 23.9°±0.2°, 25.0°±0.2°, and 31.4°±0.2°. In some embodiments, Compound 1 L-Tartrate Form LB has at least two characteristic XRPD peaks in terms of 2θ selected from 6.3°±0.2°, 12.5°±0.2°, 18.1°±0.2°, 18.7°±0.2°, 20.0°±0.2°, 23.9°±0.2°, 24.2°±0.2°, 25.0°±0.2°, and 31.4°±0.20.

In some embodiments, Compound 1 L-Tartrate Form LB has at least three characteristic XRPD peaks in terms of 2θ selected from 6.3°±0.2°, 12.5°±0.2°, 18.1°±0.2°, 18.7°±0.2°, 23.9°±0.2°, 25.0°±0.2°, and 31.4°±0.2°. In some embodiments, Compound 1 L-Tartrate Form LB has at least three characteristic XRPD peaks in terms of 2θ selected from 6.3°±0.2°, 12.5°±0.2°, 18.1°±0.2°, 18.7°±0.2°, 20.0°±0.2°, 23.9°±0.2°, 24.2°±0.2°, 25.0°±0.2°, and 31.4°±0.20.

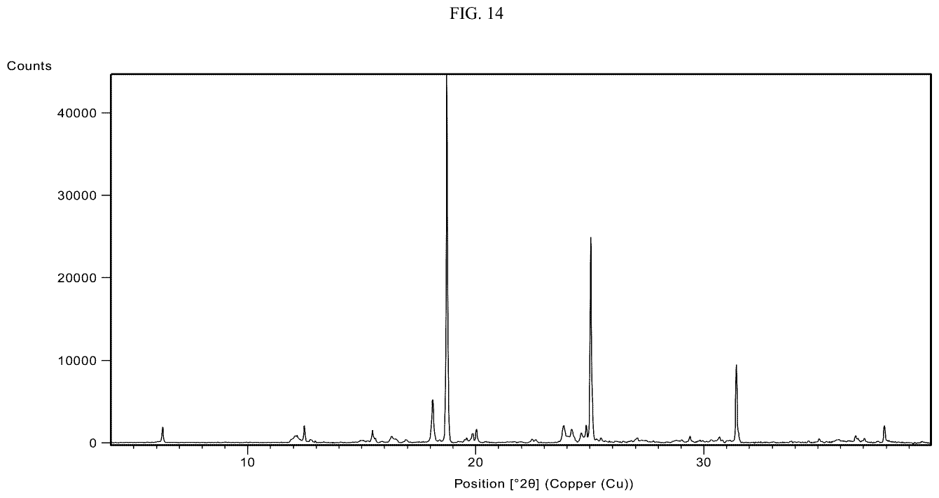

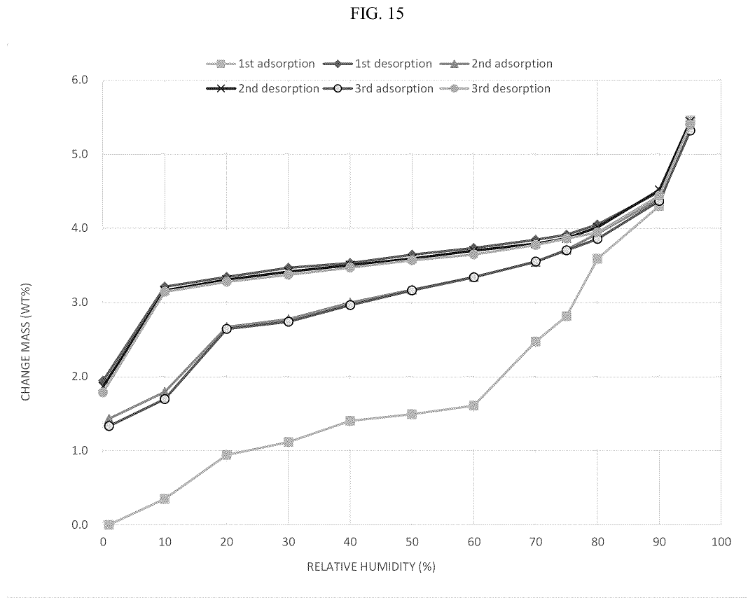

In some embodiments, Compound 1 L-Tartrate Form LB has an XRPD pattern with characteristic peaks as substantially shown in FIG. 14 (FIG. 14). In some embodiments, Compound L-Tartrate Form LB has a DVS isotherm as substantially shown in FIG. 15 (FIG. 15).

In some embodiments, Compound 1 L-Tartrate Form LB can be isolated with a crystalline purity of at least about 80%, about 85%, about 90%, about 95%, about 96%, about 97%, about 98%, or about 99%. In some embodiments, Compound 1 L-Tartrate Form LB can be isolated with a crystalline purity greater than about 99%. In some embodiments, Compound 1 L-Tartrate Form LB can be isolated with a crystalline purity greater than about 99.9%.

In some embodiments, provided is Compound 1 L-tartrate Form LB prepared by isolating Compound 1 L-tartrate Form LB from a mixture of Compound 1, L-tartaric acid, and S1, wherein S1 is a solvent. In some embodiments, S1 is an organic solvent. In some embodiments, S1 is C₁₋₆ alkyl alcohol. In some embodiments, S1 is a C₁₋₆ alkyl ketone. In some embodiments, S1 is a mixture of organic solvents. In some embodiments, S1 is a mixture of a C₁₋₆ alkyl alcohol and a C₁₋₆ alkyl ketone. In some embodiments, S1 is a mixture of methanol and acetone.

### Compound 1 L-Tartrate Form LC

In some embodiments, provided is (R)-1-(8-fluoroisochroman-1-yl)-N-methylmethanamine L-Tartrate Form LC (Compound 1 L-tartrate Form LC). In some embodiments, Compound 1 L-Tartrate Form LC is crystalline.

In some embodiments, Compound 1 L-Tartrate Form LC has characteristic XRPD peaks in terms of 2θ selected from 12.2°±0.2°, 16.5°±0.2°, and 24.8°±0.2°. In some embodiments, Compound 1 L-Tartrate Form LC has a characteristic XRPD peak in terms of 2θ at 12.2°±0.2°. In some embodiments, Compound 1 L-Tartrate Form LC has a characteristic XRPD peak in terms of 2θ at 16.5°±0.2°. In some embodiments, Compound 1 L-Tartrate Form LC has a characteristic XRPD peak in terms of 2θ at 24.8°±0.2°.

In some embodiments, Compound 1 L-Tartrate Form LC has at least one characteristic XRPD peak in terms of 2θ selected from 12.2°±0.2°, 15.4°±0.2°, 16.5°±0.2°, 18.7°±0.2°, 19.8°±0.2°, 22.6°±0.2°, 24.8°±0.2°, and 25.5°±0.2°. In some embodiments, Compound 1 L-Tartrate Form LC has at least one characteristic XRPD peak in terms of 2θ selected from 12.2°±0.2°, 12.8°±0.2°, 15.4°±0.2°, 16.5°±0.2°, 18.7°±0.2°, 19.8°±0.2°, 20.0°±0.20, 22.4°±0.20, 22.6°±0.20, 24.8°±0.20, 25.0°±0.20, 25.5°±0.20, and 27.1°±0.20.

In some embodiments, Compound 1 L-Tartrate Form LC has at least two characteristic XRPD peaks in terms of 2θ selected from 12.2°±0.2°, 15.4°±0.2°, 16.5°±0.2°, 18.7°±0.2°, 19.8°±0.2°, 22.6°±0.2°, 24.8°±0.2°, and 25.5°±0.2°. In some embodiments, Compound 1 L-Tartrate Form LC has at least two characteristic XRPD peaks in terms of 2θ selected from 12.2°±0.2°, 12.8°±0.2°, 15.4°±0.2°, 16.5°±0.2°, 18.7°±0.2°, 19.8°±0.2°, 20.0°±0.20, 22.4°±0.20, 22.6°±0.20, 24.8°±0.20, 25.0°±0.20, 25.5°±0.20, and 27.1°±0.20.

In some embodiments, Compound 1 L-Tartrate Form LC has at least three characteristic XRPD peaks in terms of 2θ selected from 12.2°±0.2°, 15.4°±0.2°, 16.5°±0.2°, 18.7°±0.2°, 19.8°±0.2°, 22.6°±0.2°, 24.8°±0.2°, and 25.5°±0.2°. In some embodiments, Compound 1 L-Tartrate Form LC has at least three characteristic XRPD peaks in terms of 2θ selected from 12.2°±0.2°, 12.8°±0.2°, 15.4°±0.2°, 16.5°±0.2°, 18.7°±0.2°, 19.8°±0.2°, 20.0°±0.20, 22.4°±0.20, 22.6°±0.20, 24.8°±0.20, 25.0°±0.20, 25.5°±0.20, and 27.1°±0.20.

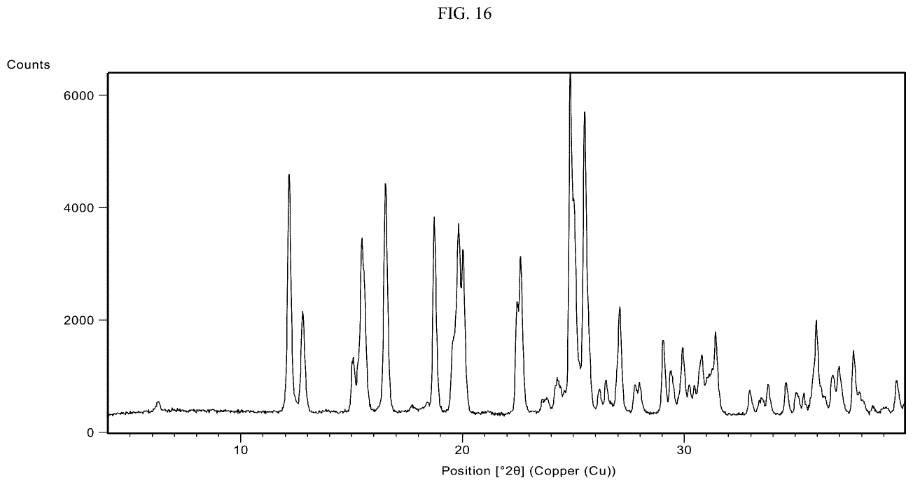

In some embodiments, Compound 1 L-Tartrate Form LC has an XRPD pattern with characteristic peaks as substantially shown in FIG. 16 (FIG. 16).

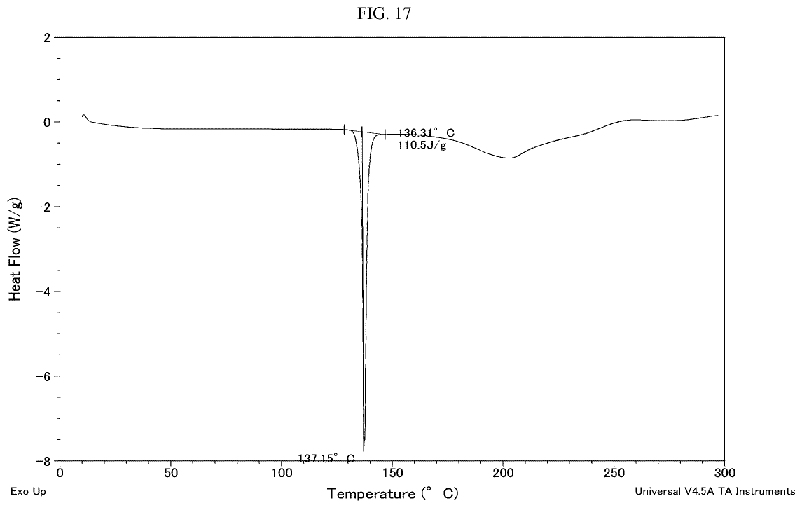

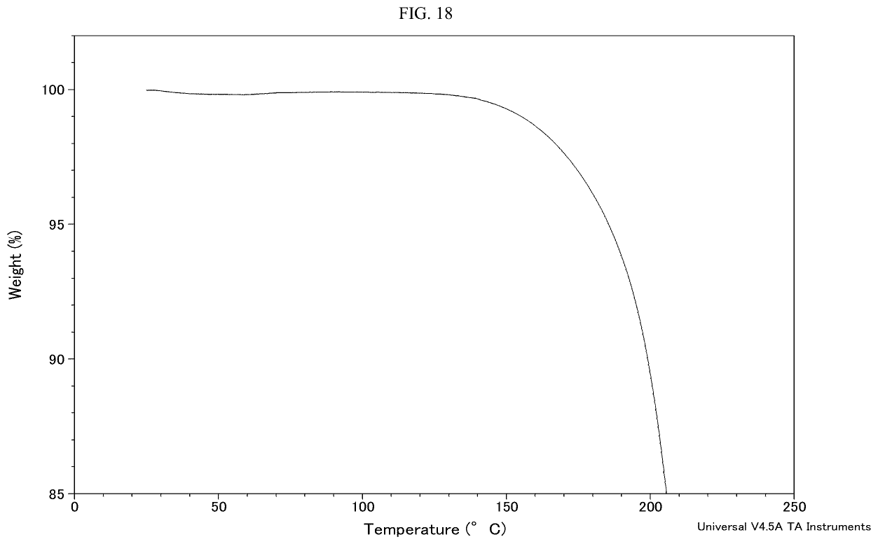

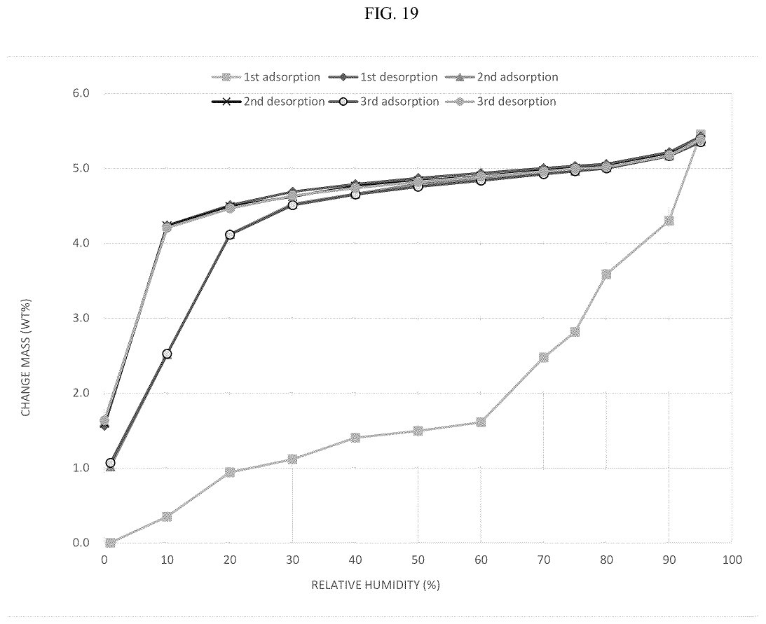

In some embodiments, Compound 1 L-Tartrate Form LC has an endotherm peak at a temperature of about 137° C. In some embodiments, Compound 1 L-Tartrate Form LC has a DSC thermogram substantially as depicted in FIG. 17 (FIG. 17). In some embodiments, Compound 1 L-Tartrate Form LC has a TGA thermogram substantially as depicted in FIG. 18 (FIG. 18). In some embodiments, Compound 1 L-Tartrate Form LC has a DVS isotherm substantially as depicted in FIG. 19 (FIG. 19).

In some embodiments, Compound 1 L-Tartrate Form LC has at least one characteristic XRPD peak in terms of 2θ selected from 12.2°±0.2°, 16.5°±0.2°, and 24.8°±0.2°; and an endotherm peak at a temperature of about 137° C. In some embodiments, Compound 1 L-Tartrate Form LC has at least one characteristic XRPD peak in terms of 2θ selected from 12.2°±0.2°, 16.5°±0.2°, and 24.8°±0.2°; and a DSC thermogram substantially as depicted in FIG. 17 (FIG. 17). In some embodiments, Compound 1 L-Tartrate Form LC has at least one characteristic XRPD peak in terms of 2θ selected from 12.2°±0.2°, 16.5°±0.2°, and 24.8°±0.2°; and a DVS isotherm substantially as depicted in FIG. 19 (FIG. 19).

In some embodiments, Compound 1 L-Tartrate Form LC can be isolated with a crystalline purity of at least about 80%, about 85%, about 90%, about 95%, about 96%, about 97%, about 98%, or about 99%. In some embodiments, Compound 1 L-Tartrate Form LC can be isolated with a crystalline purity greater than about 99%. In some embodiments, Compound 1 L-Tartrate Form LC can be isolated with a crystalline purity greater than about 99.9%.

In some embodiments, provided is Compound 1 L-tartrate Form LB prepared by isolating Compound 1 L-tartrate Form LB from a mixture of Compound 1, L-tartaric acid, and S1, wherein S1 is a solvent. In some embodiments, S1 is an organic solvent. In some embodiments, S1 is C₁₋₆ alkyl alcohol. In some embodiments, S1 is C₁₋₆ alkyl acetate. In some embodiments, S1 is a mixture of organic solvents. In some embodiments, S1 is a mixture of a C₁₋₆ alkyl alcohol and a C₁₋₆ alkyl acetate. In some embodiments, S1 is a mixture of methanol and ethyl acetate.

### Compound 1 D-tartrate

In some embodiments, provided is (R)-1-(8-fluoroisochroman-1-yl)-N-methylmethanamine D-Tartrate (Compound 1 D-Tartrate). In some embodiments, Compound 1 D-tartrate is crystalline.

In some embodiments, Compound 1 D-Tartrate has characteristic XRPD peaks in terms of 2θ selected from 11.9°±0.2°, 16.9°±0.2°, and 17.9°±0.2°. In some embodiments, Compound 1 D-Tartrate has a characteristic XRPD peak in terms of 2θ at 11.9°±0.2°. In some embodiments, Compound 1 D-Tartrate has a characteristic XRPD peak in terms of 2θ at 16.9°±0.2°. In some embodiments, Compound 1 D-Tartrate has a characteristic XRPD peak in terms of 2θ at 17.9°±0.2°.

In some embodiments, Compound 1 D-Tartrate has characteristic XRPD peaks in terms of 2θ selected from 11.9°±0.2°, 16.9°±0.2°, 17.9°±0.2°, and 23.9°±0.2°. In some embodiments, Compound 1 D-Tartrate has at least one characteristic XRPD peak in terms of 2θ selected from 11.9°±0.2°, 16.9°±0.2°, 17.9°±0.2°, and 23.9°±0.2°.

In some embodiments, Compound 1 D-Tartrate has at least one characteristic XRPD peak in terms of 2θ selected from 11.9°±0.2°, 12.3°±0.2°, 16.1°±0.2°, 16.9°±0.2°, 17.9°±0.2°, 19.1°±0.2°, and 23.9°±0.2°. In some embodiments, Compound 1 D-Tartrate has at least one characteristic XRPD peak in terms of 2θ selected from 6.0°±0.2°, 11.9°±0.2°, 12.3°±0.2°, 13.4°±0.2°, 14.9°±0.2°, 16.1°±0.2°, 16.9°±0.2°, 17.9°±0.2°, 19.1°±0.2°, 21.6°±0.2°, 23.90°±0.20, and 24.60°±0.2°.

In some embodiments, Compound 1 D-Tartrate has at least two characteristic XRPD peaks in terms of 2θ selected from 11.9°±0.2°, 12.3°±0.2°, 16.1°±0.2°, 16.9°±0.2°, 17.9°±0.2°, 19.1°±0.2°, and 23.9°±0.2°. In some embodiments, Compound 1 D-Tartrate has at least two characteristic XRPD peaks in terms of 2θ selected from 6.0°±0.2°, 11.9°±0.2°, 12.3°±0.2°, 13.4°±0.2°, 14.9°±0.2°, 16.1°±0.2°, 16.9°±0.2°, 17.9°±0.2°, 19.10°±0.2°, 21.6°±0.2°, 23.9°±0.2°, and 24.6°±0.2°.

In some embodiments, Compound 1 D-Tartrate has at least three characteristic XRPD peaks in terms of 2θ selected from 11.9°±0.2°, 12.3°±0.2°, 16.1°±0.2°, 16.9°±0.2°, 17.9°±0.2°, 19.1°±0.2°, and 23.9°±0.2°. In some embodiments, Compound 1 D-Tartrate has at least three characteristic XRPD peaks in terms of 2θ selected from 6.0°±0.2°, 11.9°±0.2°, 12.30°±0.2°, 13.4°±0.2°, 14.9°±0.2°, 16.1°±0.2°, 16.9°±0.2°, 17.9°±0.2°, 19.10°±0.2°, 21.6°±0.2°, 23.9°±0.2°, and 24.6°±0.2°.

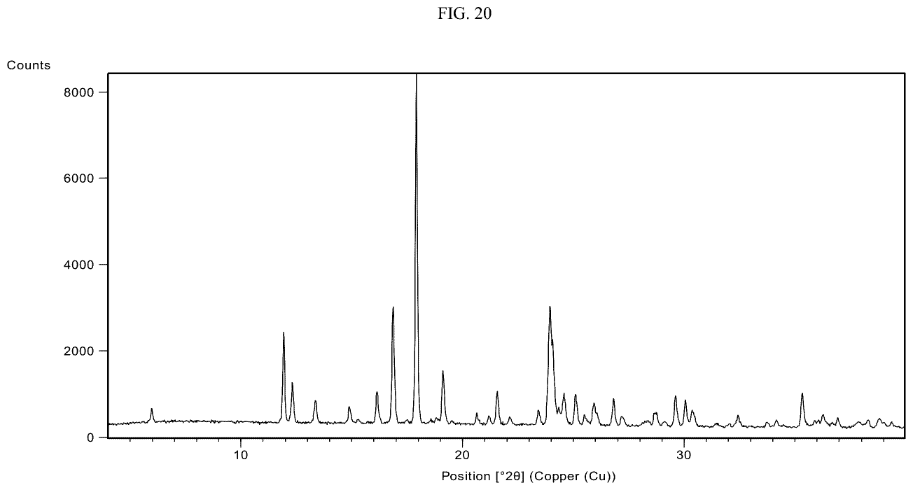

In some embodiments, Compound 1 D-Tartrate has an XRPD pattern with characteristic peaks as substantially shown in FIG. 20 (FIG. 20).

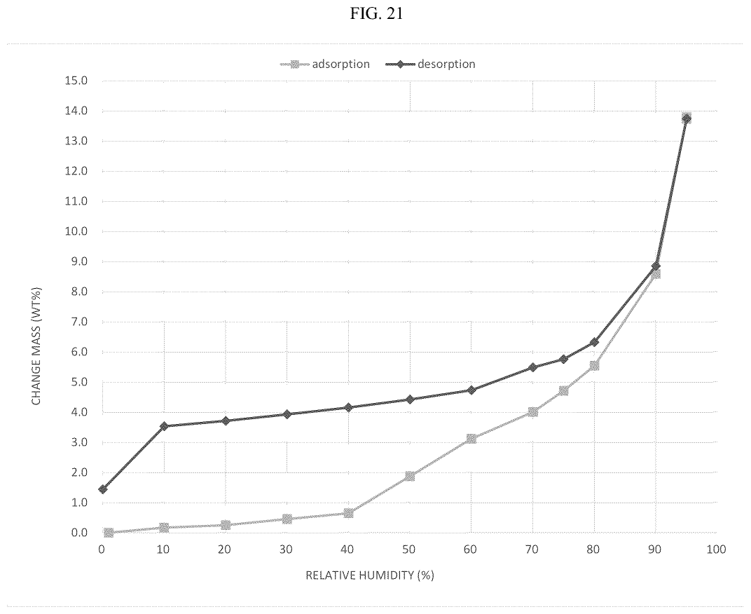

In some embodiments, Compound 1 D-Tartrate has endotherm peaks at temperatures of about 76° C. and about 153° C. In some embodiments, Compound 1 D-Tartrate has an endotherm peak at a temperature of about 76° C. In some embodiments, Compound 1 D-Tartrate has an endotherm peak at a temperature of about 153° C. In some embodiments, Compound 1 D-Tartrate has a DVS isotherm substantially as depicted in FIG. 21 (FIG. 21).

In some embodiments, Compound 1 D-Tartrate has at least one characteristic XRPD peak in terms of 2θ selected from 11.9°±0.2°, 16.9°±0.2°, 17.9°±0.2°, and 23.9°±0.2°; and has endotherm peaks at temperatures of about 76° C. and about 153° C. In some embodiments, Compound 1 D-Tartrate has at least one characteristic XRPD peak in terms of 2θ selected from 11.9°±0.2°, 16.9°±0.2°, 17.9°±0.2°, and 23.9°±0.2°; and an endotherm peak at a temperature of about 76° C. In some embodiments, Compound 1 D-Tartrate has at least one characteristic XRPD peak in terms of 2θ selected from 11.9°±0.2°, 16.9°±0.2°, 17.9°±0.2°, and 23.9°±0.2°; and an endotherm peak at a temperature of about 153° C. In some embodiments, Compound 1 D-Tartrate has at least one characteristic XRPD peak in terms of 2θ selected from 11.9°±0.2°, 16.9°±0.2°, 17.9°±0.2°, and 23.9°±0.2°; and a DVS isotherm substantially as depicted in FIG. 21 (FIG. 21).

In some embodiments, Compound 1 D-Tartrate can be isolated with a crystalline purity of at least about 80%, about 85%, about 90%, about 95%, about 96%, about 97%, about 98%, or about 99%. In some embodiments, Compound 1 D-Tartrate can be isolated with a crystalline purity greater than about 99%. In some embodiments, Compound D-Tartrate can be isolated with a crystalline purity greater than about 99.9%.

In some embodiments, provided is Compound 1 D-Tartrate prepared by isolating Compound 1 D-Tartrate from a mixture of Compound 1, D-tartaric acid, and S1, wherein S1 is a solvent. In some embodiments, S1 is an organic solvent. In some embodiments, S1 is ether. In some embodiments, S1 is C₁₋₆ alkyl acetate. In some embodiments, S1 is THF. In some embodiments, S1 is ethyl acetate.

### Compound 1 Fumarate

In some embodiments, provided is (R)-1-(8-fluoroisochroman-1-yl)-N-methylmethanamine fumarate (Compound 1 Fumarate). In some embodiments, Compound 1 Fumarate is crystalline.

In some embodiments, provided is Compound 1 Fumarate prepared by isolating Compound 1 Fumarate from a mixture of Compound 1, fumaric acid, and S1, wherein S1 is a solvent. In some embodiments, S1 is an organic solvent. In some embodiments, S1 is C₁₋₆ alkyl alcohol. In some embodiments, S1 is ether. In some embodiments, S1 is C₁₋₆ alkyl acetate. In some embodiments, S1 is a C₁₋₆ alkyl ketone. In some embodiments, S1 is a mixture of organic solvents. In some embodiments, S1 is a mixture of a C₁₋₆ alkyl alcohol and a C₁₋₆ alkyl ketone. In some embodiments, S1 is methanol. In some embodiments, S1 is THF. In some embodiments, S1 is ethyl acetate. In some embodiments, S1 is a mixture of methanol and acetone.

### Compound 1 Fumarate Form FA

In some embodiments, provided is (R)-1-(8-fluoroisochroman-1-yl)-N-methylmethanamine Fumarate Form FA (Compound 1 fumarate Form FA). In some embodiments, Compound 1 Fumarate Form FA is crystalline.

In some embodiments, Compound 1 Fumarate Form FA has characteristic XRPD peaks in terms of 2θ selected from 7.7°±0.2°, 14.2°±0.2°, and 15.2°±0.2°. In some embodiments, Compound 1 Fumarate Form FA has a characteristic XRPD peak in terms of 2θ at 7.7°±0.2°. In some embodiments, Compound 1 Fumarate Form FA has a characteristic XRPD peak in terms of 2θ at 14.2°±0.2°. In some embodiments, Compound 1 Fumarate Form FA has a characteristic XRPD peak in terms of 2θ at 15.2°±0.2°.

In some embodiments, Compound 1 Fumarate Form FA has characteristic XRPD peaks in terms of 2θ selected from 7.7°±0.2°, 15.2°±0.2°, 22.9°±0.2°, and 30.7°±0.2°. In some embodiments, Compound 1 Fumarate Form FA has at least one characteristic XRPD peak in terms of 2θ selected from 7.7°±0.2°, 15.2°±0.2°, 22.9°±0.2°, and 30.7°±0.2°.

In some embodiments, Compound 1 Fumarate Form FA has at least one characteristic XRPD peak in terms of 2θ selected from 7.7°±0.2°, 13.0°±0.2°, 14.2°±0.2°, 15.2°±0.2°, 22.9°±0.2°, 24.6°±0.2°, 26.0°±0.2°, and 30.7°±0.2°. In some embodiments, Compound 1 Fumarate Form FA has at least one characteristic XRPD peak in terms of 2θ selected from 7.7°±0.2°, 12.1°±0.2°, 13.0°±0.2°, 14.2°±0.2°, 14.6°±0.2°, 15.2°±0.2°, 18.1°±0.2°, 18.8°±0.2°, 22.9°±0.2°, 24.6°±0.2°, 26.0°±0.2°, and 30.7°±0.2°.

In some embodiments, Compound 1 Fumarate Form FA has at least two characteristic XRPD peaks in terms of 2θ selected from 7.7°±0.2°, 13.0°±0.2°, 14.2°±0.2°, 15.2°±0.2°, 22.9°±0.2°, 24.6°±0.2°, 26.0°±0.2°, and 30.7°±0.2°. In some embodiments, Compound 1 Fumarate Form FA has at least two characteristic XRPD peaks in terms of 2θ selected from 7.7°±0.2°, 12.1°±0.2°, 13.0°±0.2°, 14.2°±0.2°, 14.6°±0.2°, 15.2°±0.2°, 18.1°±0.2°, 18.8°±0.2°, 22.9°±0.2°, 24.6°±0.2°, 26.0°±0.2°, and 30.7°±0.2°.

In some embodiments, Compound 1 Fumarate Form FA has at least three characteristic XRPD peaks in terms of 2θ selected from 7.7°±0.2°, 13.0°±0.2°, 14.2°±0.2°, 15.2°±0.2°, 22.9°±0.2°, 24.6°±0.2°, 26.0°±0.2°, and 30.7°±0.2°. In some embodiments, Compound 1 Fumarate Form FA has at least three characteristic XRPD peaks in terms of 2θ selected from 7.7°±0.2°, 12.1°±0.2°, 13.0°±0.2°, 14.2°±0.2°, 14.6°±0.2°, 15.2°±0.2°, 18.1°±0.2°, 18.8°±0.2°, 22.9°±0.2°, 24.6°±0.2°, 26.0°±0.2°, and 30.7°±0.2°.

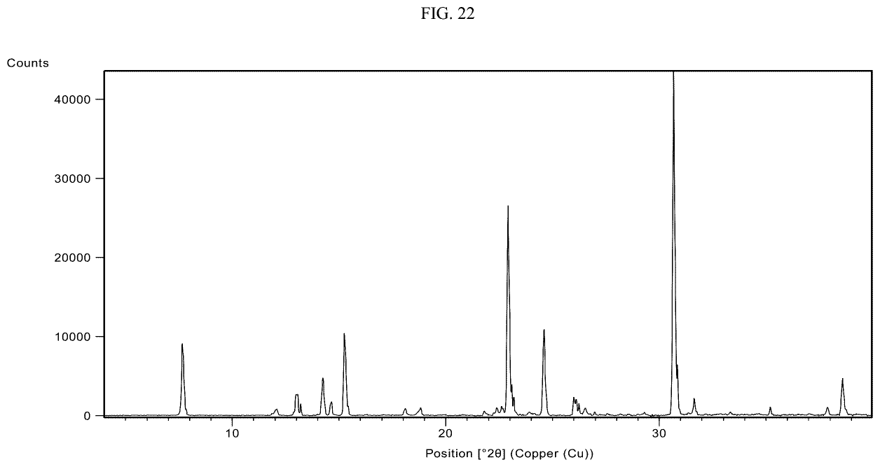

In some embodiments, Compound 1 Fumarate Form FA has an XRPD pattern with characteristic peaks as substantially shown in FIG. 22 (FIG. 22).

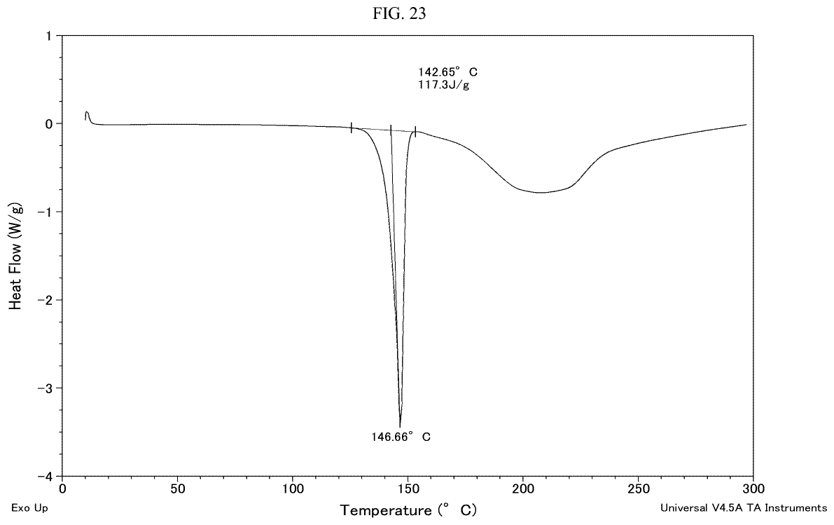

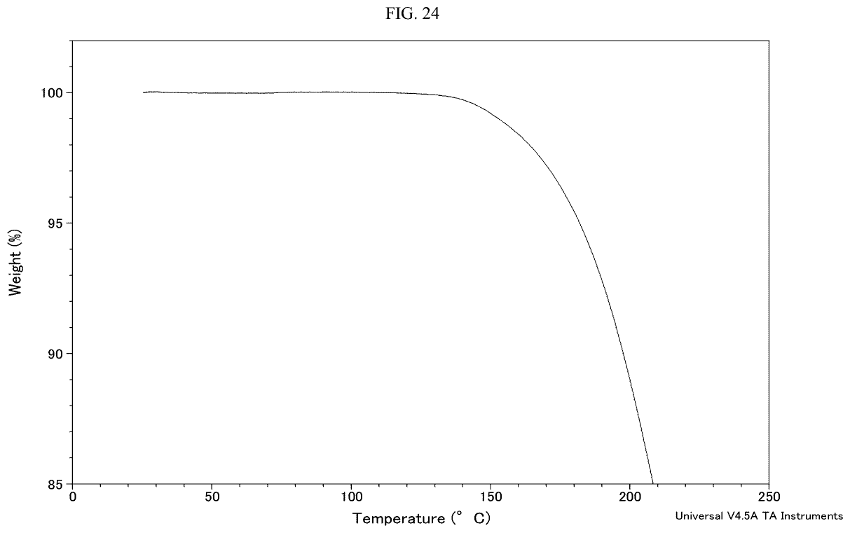

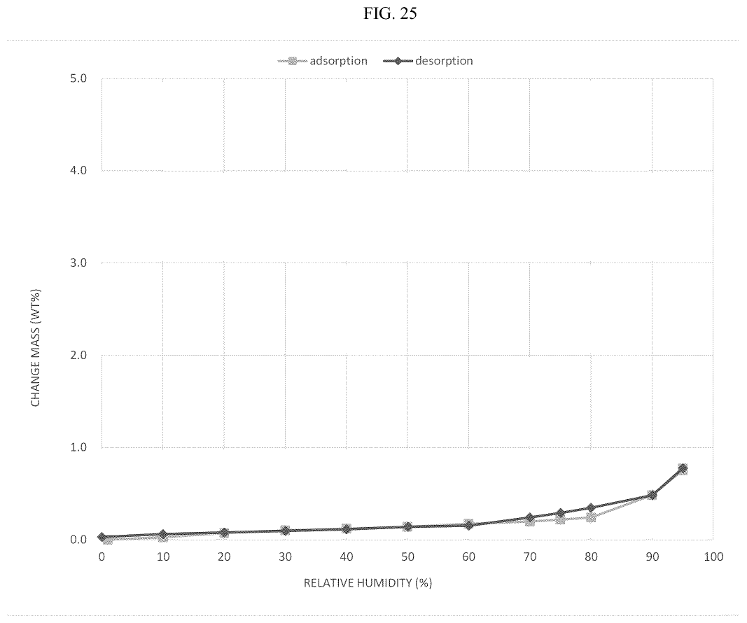

In some embodiments, Compound 1 Fumarate Form FA has an endotherm peak at a temperature of about 138° C. In some embodiments, Compound 1 Fumarate Form FA has a DSC thermogram substantially as depicted in FIG. 23 (FIG. 23). In some embodiments, Compound 1 L-Fumarate Form FA has a TGA thermogram substantially as depicted in FIG. 24 (FIG. 24). In some embodiments, Compound 1 Fumarate Form FA has a DVS isotherm substantially as depicted in FIG. 25 (FIG. 25).

In some embodiments, Compound 1 Fumarate Form FA has characteristic XRPD peaks in terms of 2θ selected from 7.7°±0.2°, 14.2°±0.2°, and 15.2°±0.2°; and an endotherm peak at a temperature of about 147° C. In some embodiments, Compound 1 Fumarate Form FA has characteristic XRPD peaks in terms of 2θ selected from 7.7°±0.2°, 14.2°±0.2°, and 15.2°±0.2°; and a DSC thermogram substantially as depicted in FIG. 23 (FIG. 23). In some embodiments, Compound 1 Fumarate Form FA has characteristic XRPD peaks in terms of 2θ selected from 7.7°±0.2°, 14.2°±0.2°, and 15.2°±0.2°; and a DVS isotherm substantially as depicted in FIG. 25 (FIG. 25).

In some embodiments, Compound 1 Fumarate Form FA can be isolated with a crystalline purity of at least about 80%, about 85%, about 90%, about 95%, about 96%, about 97%, about 98%, or about 99%. In some embodiments, Compound 1 Fumarate Form FA can be isolated with a crystalline purity greater than about 99%. In some embodiments, Compound 1 Fumarate Form FA can be isolated with a crystalline purity greater than about 99.9%.

In some embodiments, provided is Compound 1 Fumarate Form FA prepared by isolating Compound 1 Fumarate Form FA from a mixture of Compound 1, fumaric acid, and S1, wherein S1 is a solvent. In some embodiments, S1 is an organic solvent. In some embodiments, S1 is C₁₋₆ alkyl alcohol. In some embodiments, S1 is ether. In some embodiments, S1 is a C₁₋₆ alkyl ketone. In some embodiments, S1 is a mixture of organic solvents. In some embodiments, S1 is a mixture of a C₁₋₆ alkyl alcohol and a C₁₋₆ alkyl ketone. In some embodiments, S1 is methanol. In some embodiments, S1 is THF. In some embodiments, S1 is a mixture of methanol and acetone.

### Compound 1 Fumarate FB

In some embodiments, provided is (R)-1-(8-fluoroisochroman-1-yl)-N-methylmethanamine Fumarate Form FB (Compound 1 fumarate Form FB). In some embodiments, Compound 1 Fumarate Form FB is crystalline.

In some embodiments, Compound 1 Fumarate Form FB has characteristic XRPD peaks in terms of 2θ selected from 6.7°±0.2°, 13.8°±0.2°, and 20.2°±0.2°. In some embodiments, Compound 1 Fumarate Form FB has a characteristic XRPD peak in terms of 2θ at 6.7°±0.2°. In some embodiments, Compound 1 Fumarate Form FB has a characteristic XRPD peak in terms of 2θ at 13.8°±0.2°. In some embodiments, Compound 1 Fumarate Form FB has a characteristic XRPD peak in terms of 2θ at 20.2°±0.2°. In some embodiments, Compound 1 Fumarate Form FB has a characteristic XRPD peak in terms of 2θ at 27.0°±0.2°.

In some embodiments, Compound 1 Fumarate Form FB has at least one characteristic XRPD peak in terms of 2θ selected from 6.7°±0.2°, 13.4°±0.2°, 13.8°±0.2°, 20.2°±0.2°, 23.5°±0.2°, 25.1°±0.2°, 27.0°±0.2°, and 29.6°±0.2°. In some embodiments, Compound 1 Fumarate Form FB has at least one characteristic XRPD peak in terms of 2θ selected from 6.7°±0.2°, 13.4°±0.2°, 13.8°±0.2°, 20.2°±0.2°, 23.1°±0.2°, 23.5°±0.2°, 24.2°±0.2°, 25.1°±0.2°, 27.0°±0.2°, and 29.6°±0.2°.

In some embodiments, Compound 1 Fumarate Form FB has at least two characteristic XRPD peaks in terms of 2θ selected from 6.7°±0.2°, 13.4°±0.2°, 13.8°±0.2°, 20.2°±0.2°, 23.5°±0.2°, 25.1°±0.2°, 27.0°±0.2°, and 29.6°±0.2°. In some embodiments, Compound 1 Fumarate Form FB has at least two characteristic XRPD peaks in terms of 2θ selected from 6.7°±0.2°, 13.4°±0.2°, 13.8°±0.2°, 20.2°±0.2°, 23.1°±0.2°, 23.5°±0.2°, 24.2° 0.20, 25.1°±0.2°, 27.0°±0.2°, and 29.6°±0.2°.

In some embodiments, Compound 1 Fumarate Form FB has at least three characteristic XRPD peaks in terms of 2θ selected from 6.7°±0.2°, 13.4°±0.2°, 13.8°±0.2°, 20.2°±0.2°, 23.5°±0.2°, 25.1°±0.2°, 27.0°±0.2°, and 29.6°±0.2°. In some embodiments, Compound 1 Fumarate Form FB has at least three characteristic XRPD peaks in terms of 2θ selected from 6.7°±0.2°, 13.4°±0.2°, 13.8°±0.2°, 20.2°±0.2°, 23.1°±0.2°, 23.5°±0.2°, 24.2°±0.2°, 25.1°±0.2°, 27.0°±0.2°, and 29.6°±0.2°.

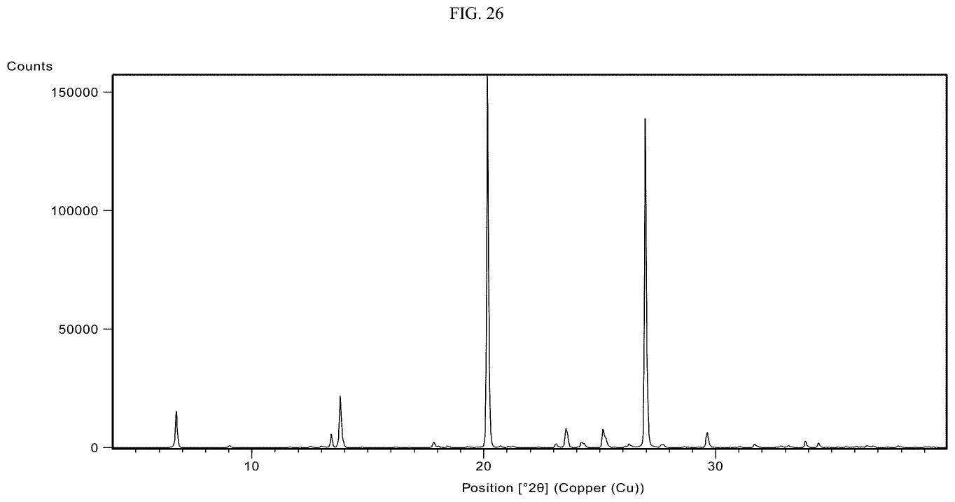

In some embodiments, Compound 1 Fumarate Form FB has an XRPD pattern with characteristic peaks as substantially shown in FIG. 26 (FIG. 26).

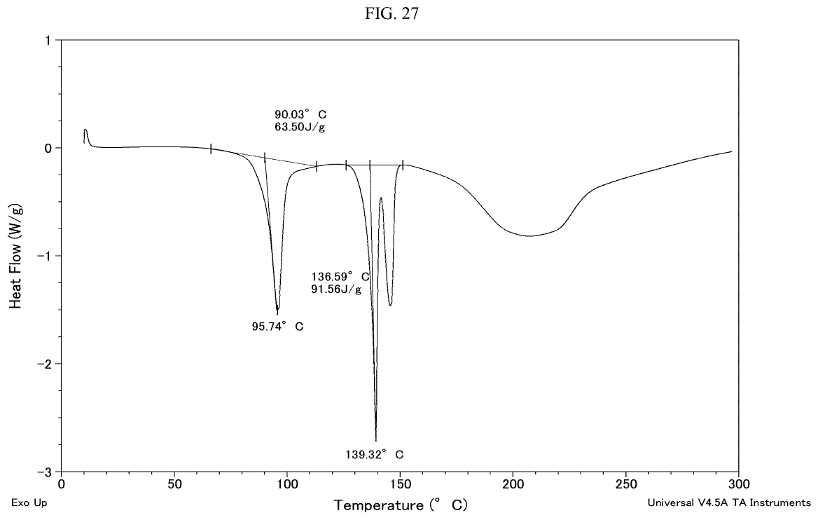

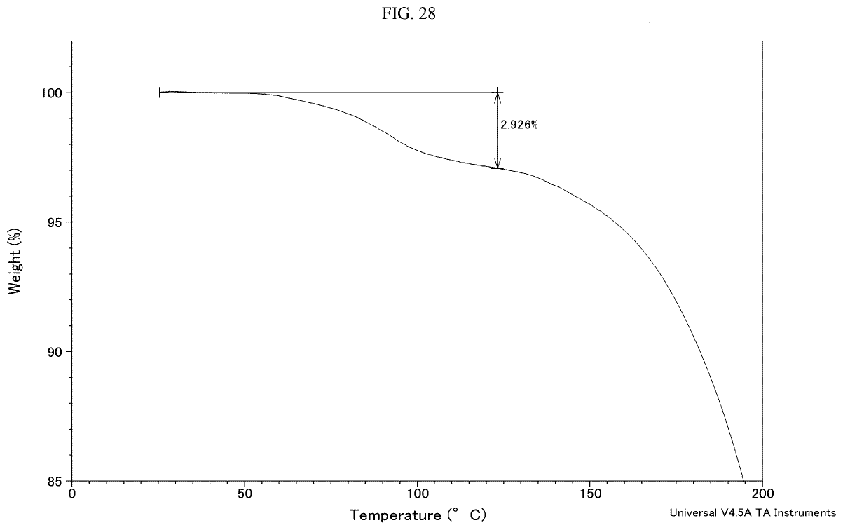

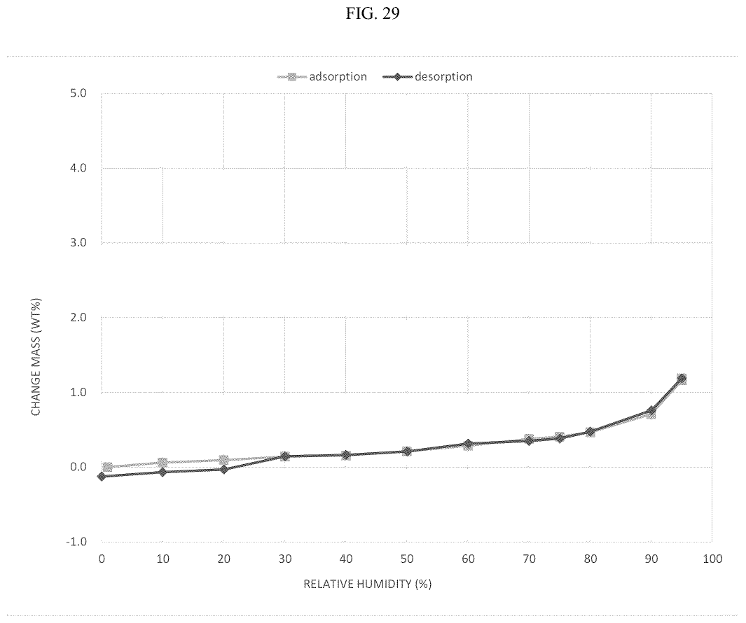

In some embodiments, Compound 1 Fumarate Form FB has endotherm peaks at temperatures of about 96° C., about 139° C., and about 146° C. In some embodiments, Compound 1 Fumarate Form FB has an endotherm peak at a temperature of about 96° C. In some embodiments, Compound 1 Fumarate Form FB has an endotherm peak at a temperature of about 139° C. In some embodiments, Compound 1 Fumarate Form FB has an endotherm peak at a temperature of about 146° C. In some embodiments, Compound 1 Fumarate Form FB has a DSC thermogram substantially as depicted in FIG. 27 (FIG. 27). In some embodiments, Compound 1 Fumarate Form FB has a TGA thermogram substantially as depicted in FIG. 28 (FIG. 28). In some embodiments, Compound 1 Fumarate Form FB has a DVS isotherm substantially as depicted in FIG. 29 (FIG. 29).

In some embodiments, Compound 1 Fumarate Form FB has at least one characteristic XRPD peak in terms of 2θ selected from 6.7°±0.2°, 13.8°±0.2°, and 20.2°±0.2°; and endotherm peaks at temperatures of about 96° C., about 139° C., and about 146° C. In some embodiments, Compound 1 Fumarate Form FB has at least one characteristic XRPD peak in terms of 2θ selected from 6.7°±0.2°, 13.8°±0.2°, and 20.2°±0.2°; and an endotherm peak at a temperature of about 96° C. In some embodiments, Compound 1 Fumarate Form FB has at least one characteristic XRPD peak in terms of 2θ selected from 6.7°±0.2°, 13.8°±0.2°, and 20.2°±0.2°; and an endotherm peak at a temperature of about 139° C. In some embodiments, Compound 1 Fumarate Form FB has at least one characteristic XRPD peak in terms of 2θ selected from 6.7°±0.2°, 13.8°±0.2°, and 20.2°±0.2°; and an endotherm peak at a temperature of about 146° C. In some embodiments, Compound 1 Fumarate Form FB has at least one characteristic XRPD peak in terms of 2θ selected from 6.7°±0.2°, 13.8°±0.2°, and 20.2°±0.2°; and a DSC thermogram substantially as depicted in FIG. 27 (FIG. 27). In some embodiments, Compound 1 Fumarate Form FB has at least one characteristic XRPD peak in terms of 2θ selected from 6.7°±0.2°, 13.8°±0.2°, and 20.2°±0.2°; and a DVS isotherm substantially as depicted in FIG. 29 (FIG. 29).

In some embodiments, Compound 1 Fumarate Form FB can be isolated with a crystalline purity of at least about 80%, about 85%, about 90%, about 95%, about 96%, about 97%, about 98%, or about 99%. In some embodiments, Compound 1 Fumarate Form FB can be isolated with a crystalline purity greater than about 99%. In some embodiments, Compound 1 Fumarate Form FB can be isolated with a crystalline purity greater than about 99.9%.

In some embodiments, provided is Compound 1 Fumarate Form FB prepared by isolating Compound 1 Fumarate Form FB from a mixture of Compound 1, fumaric acid, and S1, wherein S1 is a solvent. In some embodiments, S1 is an organic solvent. In some embodiments, S1 is C₁₋₆ alkyl acetate. In some embodiments, S1 is ethyl acetate.

### Compound 1 Citrate

In some embodiments, provided is (R)-1-(8-fluoroisochroman-1-yl)-N-methylmethanamine citrate. In some embodiments, Compound 1 Citrate is crystalline. In some embodiments, Compound 1 citrate has characteristic XRPD peaks in terms of 2θ selected from 6.5°±0.2°, 15.5°±0.2°, and 20.4°±0.2°. In some embodiments, Compound 1 Citrate has a characteristic XRPD peak in terms of 2θ at 6.5°±0.2°. In some embodiments, Compound 1 Citrate has a characteristic XRPD peak in terms of 2θ at 15.5°±0.2°. In some embodiments, Compound 1 Citrate has a characteristic XRPD peak in terms of 2θ at 20.4°±0.2°.

In some embodiments, Compound 1 Citrate has at least one characteristic XRPD peak in terms of 2θ selected from 6.5°±0.2°, 10.2°±0.2°, 13.0°±0.2°, 14.5°±0.2°, 15.5°±0.2°, 17.8°±0.2°, 19.4°±0.2°, and 20.4°±0.2°. In some embodiments, Compound 1 Citrate has at least one characteristic XRPD peak in terms of 2θ selected from 6.5°±0.2°, 10.2°±0.2°, 13.0°±0.2°, 14.5°±0.2°, 15.5°±0.2°, 16.5°±0.2°, 17.3°±0.2°, 17.8°±0.2°, 19.4°±0.2°, 20.4°±0.2°, 20.8°±0.2°, 21.2°±0.2°, 21.5°±0.2°, 22.00°±0.2°, 23.1°±0.2°, and 26.0°±0.2°.

In some embodiments, Compound 1 Citrate has at least two characteristic XRPD peaks in terms of 2θ selected from 6.5°±0.2°, 10.2°±0.2°, 13.0°±0.2°, 14.5°±0.2°, 15.5°±0.2°, 17.8°±0.2°, 19.4°±0.2°, and 20.4°±0.2°. In some embodiments, Compound 1 Citrate has at least two characteristic XRPD peaks in terms of 2θ selected from 6.5°±0.2°, 10.2°±0.2°, 13.0°±0.2°, 14.5°±0.2°, 15.5°±0.2°, 16.5°±0.2°, 17.3°±0.2°, 17.8°±0.2°, 19.4°±0.2°, 20.4°±0.2°, 20.8°±0.2°, 21.2°±0.2°, 21.5°±0.2°, 22.0°±0.2°, 23.1°±0.2°, and 26.0°±0.2°.

In some embodiments, Compound 1 Citrate has at least three characteristic XRPD peaks in terms of 2θ selected from 6.5°±0.2°, 10.2°±0.2°, 13.0°±0.2°, 14.5°±0.2°, 15.5°±0.2°, 17.8°±0.2°, 19.4°±0.2°, and 20.4°±0.2°. In some embodiments, Compound 1 Citrate has at least three characteristic XRPD peaks in terms of 2θ selected from 6.5°±0.2°, 10.2°±0.2°, 13.0°±0.2°, 14.5°±0.2°, 15.5°±0.2°, 16.5°±0.2°, 17.3°±0.2°, 17.8°±0.2°, 19.4°±0.2°, 20.4°±0.2°, 20.8°±0.2°, 21.2°±0.2°, 21.5°±0.2°, 22.0°±0.2°, 23.1°±0.2°, and 26.0°±0.2°.

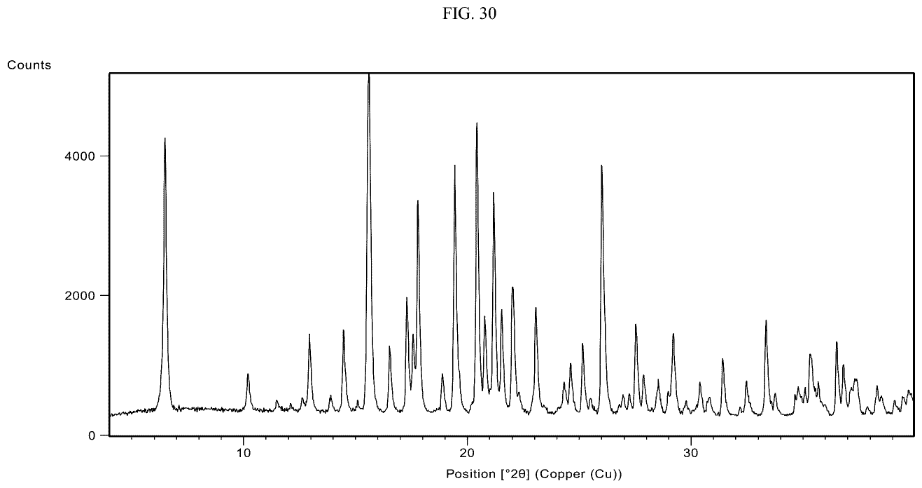

In some embodiments, Compound 1 Citrate has an XRPD pattern with characteristic peaks as substantially shown in FIG. 30 (FIG. 30).

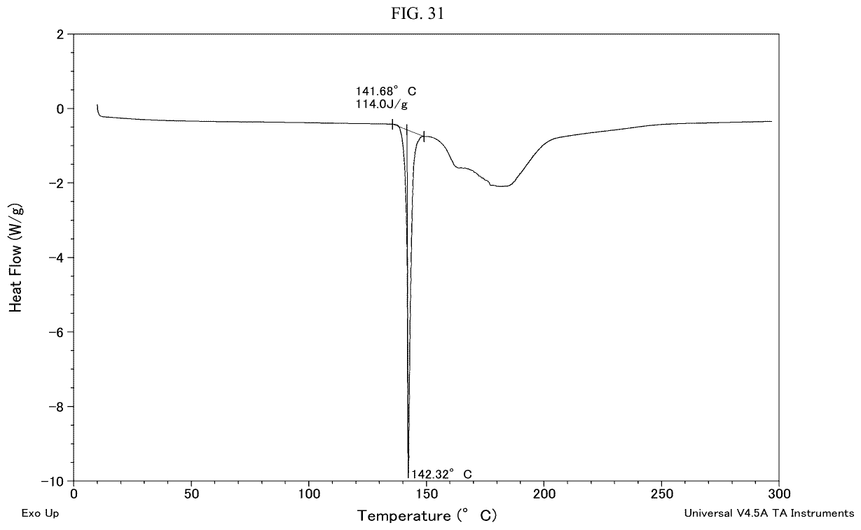

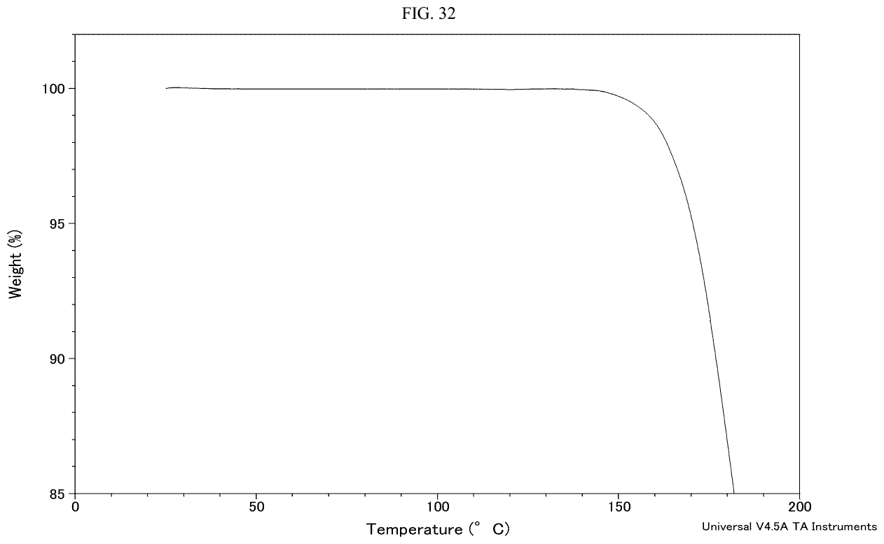

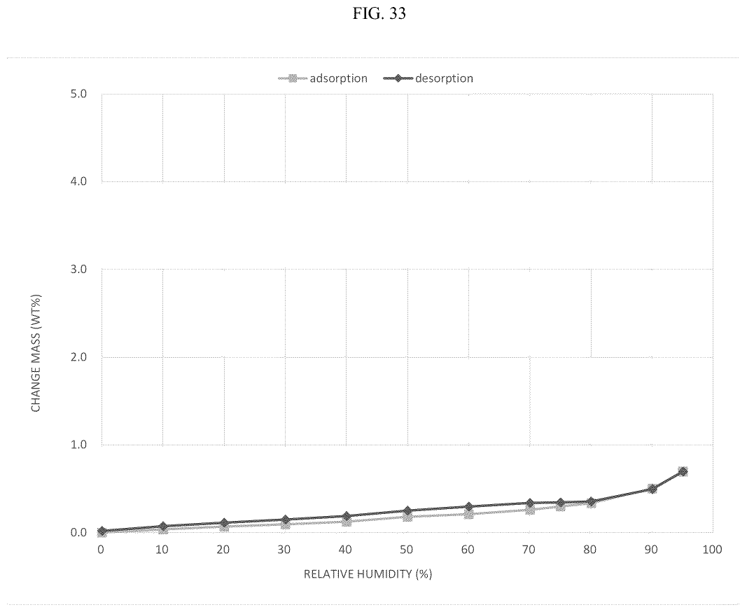

In some embodiments, Compound 1 Citrate has an endotherm peak at a temperature of about 142° C. In some embodiments, Compound 1 Citrate has a DSC thermogram substantially as depicted in FIG. 31 (FIG. 31). In some embodiments, Compound 1 Citrate has a TGA thermogram substantially as depicted in FIG. 32 (FIG. 32). In some embodiments, Compound 1 citrate has a DVS isotherm substantially as depicted in FIG. 33 (FIG. 33).

In some embodiments, Compound 1 Citrate has at least one characteristic XRPD peak in terms of 2θ selected from 6.5°±0.2°, 15.5°±0.2°, and 20.4°±0.2°; and an endotherm peak at a temperature of about 142° C. In some embodiments, Compound 1 Citrate has at least one characteristic XRPD peak in terms of 2θ selected from 6.5°±0.2°, 15.5°±0.2°, and 20.4°±0.2°; and a DSC thermogram substantially as depicted in FIG. 31 (FIG. 31). In some embodiments, Compound 1 Citrate has at least one characteristic XRPD peak in terms of 2θ selected from 6.5°±0.2°, 15.5°±0.2°, and 20.4°±0.2°; and a DVS isotherm substantially as depicted in FIG. 33 (FIG. 33).

In some embodiments, Compound 1 Citrate can be isolated with a crystalline purity of at least about 80%, about 85%, about 90%, about 95%, about 96%, about 97%, about 98%, or about 99%. In some embodiments, Compound 1 Citrate can be isolated with a crystalline purity greater than about 99%. In some embodiments, Compound 1 Citrate can be isolated with a crystalline purity greater than about 99.9%.

In some embodiments, provided is Compound 1 Citrate prepared by isolating Compound 1 citrate from a mixture of Compound 1, citric acid, and S1, wherein S1 is a solvent. In some embodiments, S1 is an organic solvent. In some embodiments, S1 is C₁₋₆ alkyl alcohol. In some embodiments, S1 is ether. In some embodiments, S1 is C₁₋₆ alkyl acetate. In some embodiments, S1 is a C₁₋₆ alkyl ketone. In some embodiments, S1 is a mixture of organic solvents. In some embodiments, S1 is a mixture of a C₁₋₆ alkyl alcohol and a C₁₋₆ alkyl ketone. In some embodiments, S1 is a mixture of a C₁₋₆ alkyl alcohol and a C₁₋₆ alkyl acetate. In some embodiments, S1 is ethyl acetate. In some embodiments, S1 is a mixture of methanol and acetone. In some embodiments, S1 is THF. In some embodiments, S1 is a mixture of methanol and ethyl acetate.

### Compound 1 Succinate

In some embodiments, provided is (R)-1-(8-fluoroisochroman-1-yl)-N-methylmethanamine succinate. In some embodiments, Compound 1 Succinate is crystalline.

In some embodiments, Compound 1 Succinate has characteristic XRPD peaks in terms of 2θ selected from 6.6°±0.2°, 12.8°±0.2°, and 13.9°±0.2°. In some embodiments, Compound 1 Succinate has a characteristic XRPD peak in terms of 2θ at 6.6°±0.2°. In some embodiments, Compound 1 Succinate has a characteristic XRPD peak in terms of 2θ at 12.8°±0.2°. In some embodiments, Compound 1 Succinate has a characteristic XRPD peak in terms of 2θ at 13.9°±0.2°.

In some embodiments, Compound 1 Succinate has characteristic XRPD peaks in terms of 2θ selected from 12.8°±0.2°, 13.9°±0.2°, 19.8°±0.2°, and 26.5°±0.2°. In some embodiments, Compound 1 Succinate has at least one characteristic XRPD peak in terms of 2θ selected from 12.8°±0.2°, 13.9°±0.2°, 19.8°±0.2°, and 26.5°±0.2°.

In some embodiments, Compound 1 Succinate has at least one characteristic XRPD peak in terms of 2θ selected from 12.8°±0.2°, 13.9°±0.2°, 16.0°±0.2°, 19.2°±0.2°, 19.8°±0.2°, 21.1°±0.2°, 22.9°±0.2°, 23.3°±0.2°, 25.4°±0.2°, and 26.5°±0.2°. In some embodiments, Compound 1 Succinate has at least one characteristic XRPD peak in terms of 2θ selected from 6.6°±0.2°, 9.0°±0.2°, 11.5°±0.2°, 12.8°±0.2°, 13.9°±0.2°, 16.0°±0.2°, 19.2°±0.2°, 19.8°±0.2°, 21.1°±0.2°, 22.9°±0.2°, 23.3°±0.2°, 25.2°±0.2°, 25.4°±0.2°, and 26.5°±0.2°.

In some embodiments, Compound 1 Succinate has at least two characteristic XRPD peaks in terms of 2θ selected from 12.8°±0.2°, 13.9°±0.2°, 16.0°±0.2°, 19.2°±0.2°, 19.8°±0.2°, 21.1°±0.2°, 22.9°±0.2°, 23.3°±0.2°, 25.4°±0.2°, and 26.5°±0.2°. In some embodiments, Compound 1 Succinate has at least two characteristic XRPD peaks in terms of 2θ selected from 6.6°±0.2°, 9.0°±0.2°, 11.5°±0.2°, 12.8°±0.2°, 13.9°±0.2°, 16.0°±0.2°, 19.2°±0.2°, 19.8°±0.2°, 21.1°±0.2°, 22.9°±0.2°, 23.3°±0.2°, 25.2°±0.2°, 25.4°±0.2°, and 26.5°±0.2°.

In some embodiments, Compound 1 Succinate has at least three characteristic XRPD peaks in terms of 2θ selected from 12.8°±0.2°, 13.9°±0.2°, 16.0°±0.2°, 19.2°±0.2°, 19.8°±0.2°, 21.1°±0.2°, 22.9°±0.2°, 23.3°±0.2°, 25.4°±0.2°, and 26.5°±0.2°. In some embodiments, Compound 1 Succinate has at least three characteristic XRPD peaks in terms of 2θ selected from 6.6°±0.2°, 9.0°±0.2°, 11.5°±0.2°, 12.8°±0.2°, 13.9°±0.2°, 16.0°±0.2°, 19.2°±0.2°, 19.8°±0.2°, 21.1°±0.2°, 22.9°±0.2°, 23.3°±0.2°, 25.2°±0.2°, 25.4°±0.2°, and 26.5°±0.2°.

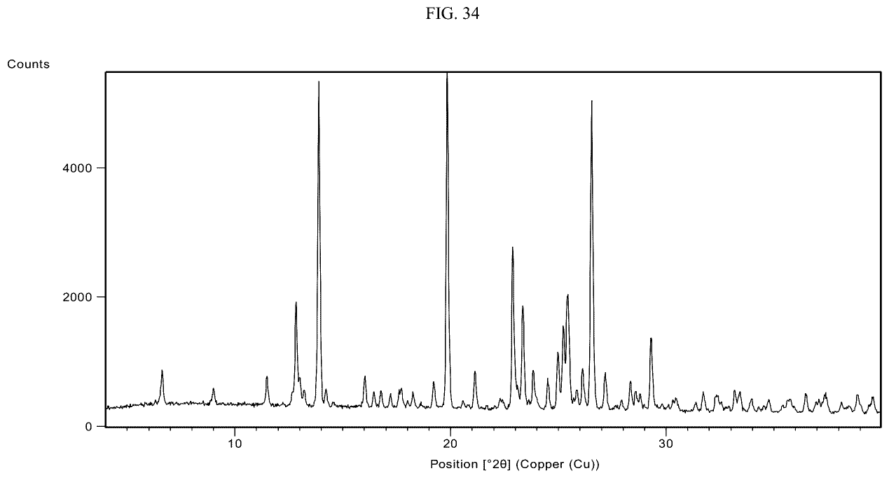

In some embodiments, Compound 1 Succinate has an XRPD pattern with characteristic peaks as substantially shown in FIG. 34 (FIG. 34).

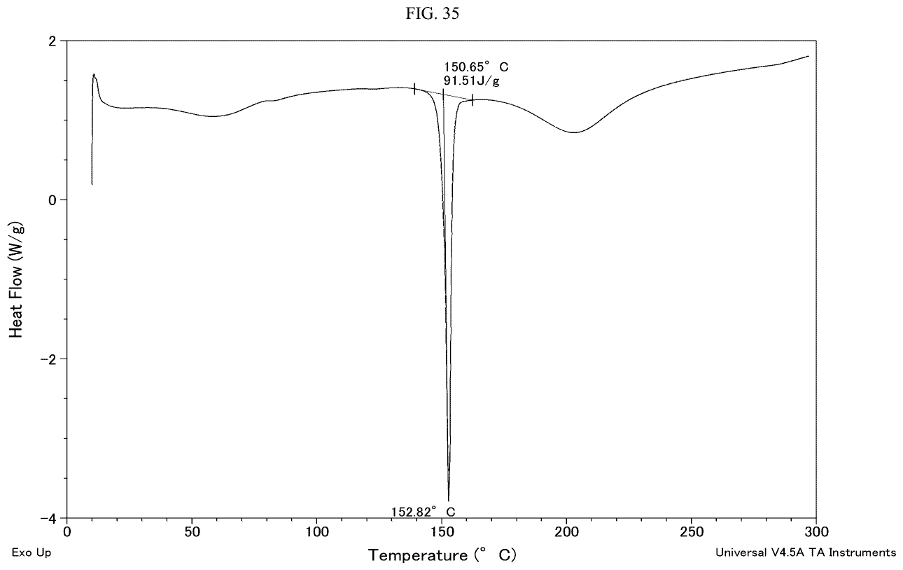

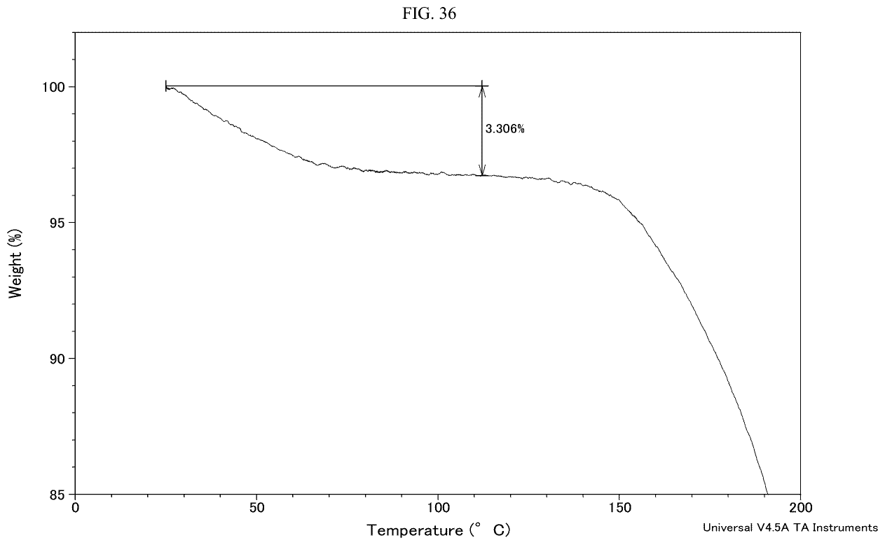

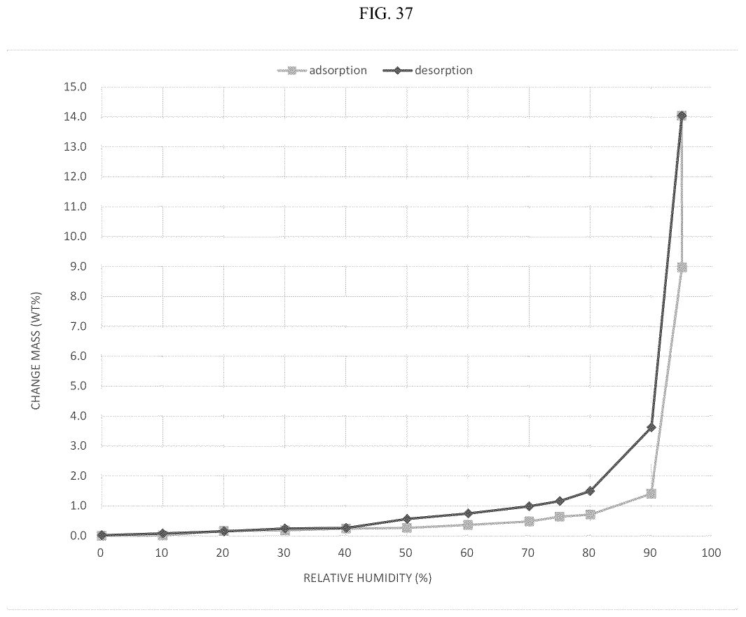

In some embodiments, Compound 1 Succinate has an endotherm peak at a temperature of about 153° C. In some embodiments, Compound 1 Succinate has a DSC thermogram substantially as depicted in FIG. 35 (FIG. 35). In some embodiments, Compound 1 Succinate has a TGA thermogram substantially as depicted in FIG. 36 (FIG. 36). In some embodiments, Compound 1 Succinate has a DVS isotherm substantially as depicted in FIG. 37 (FIG. 37).

In some embodiments, Compound 1 Succinate has at least one characteristic XRPD peak in terms of 2θ selected from 12.8°±0.2°, 13.9°±0.2°, 19.8°±0.2°, and 26.5°±0.2°; and an endotherm peak at a temperature of about 153° C. In some embodiments, Compound 1 succinate has at least one characteristic XRPD peak in terms of 2θ selected from 12.8°±0.2°, 13.9°±0.2°, 19.8°±0.2°, and 26.5°±0.2°; and a DSC thermogram substantially as depicted in FIG. 35 (FIG. 35). In some embodiments, Compound 1 Succinate has at least one characteristic XRPD peak in terms of 2θ selected from 12.8°±0.2°, 13.9°±0.2°, 19.8°±0.2°, and 26.5°±0.2°; and a DVS isotherm substantially as depicted in FIG. 37 (FIG. 37).

In some embodiments, Compound 1 Succinate can be isolated with a crystalline purity of at least about 80%, about 85%, about 90%, about 95%, about 96%, about 97%, about 98%, or about 99%. In some embodiments, Compound 1 Succinate can be isolated with a crystalline purity greater than about 99%. In some embodiments, Compound 1 Succinate can be isolated with a crystalline purity greater than about 99.9%.

In some embodiments, provided is Compound 1 Succinate prepared by isolating Compound 1 succinate from a mixture of Compound 1, succinic acid, and S1, wherein S1 is a solvent. In some embodiments, S1 is an organic solvent. In some embodiments, S1 is ether. In some embodiments, S1 is C₁₋₆ alkyl acetate. In some embodiments, S1 is THF. In some embodiments, S1 is ethyl acetate.

### Compound 1 Glutarate

In some embodiments, provided is (R)-1-(8-fluoroisochroman-1-yl)-N-methylmethanamine Glutarate. In some embodiments, Compound 1 Glutarate is crystalline.

In some embodiments, Compound 1 Glutarate has characteristic XRPD peaks in terms of 2θ selected from 9.1°±0.2°, 10.6°±0.2°, and 18.2°±0.2°. In some embodiments, Compound 1 Glutarate has a characteristic XRPD peak in terms of 2θ at 9.1°±0.2°. In some embodiments, Compound 1 Glutarate has a characteristic XRPD peak in terms of 2θ at 10.6°±0.2°. In some embodiments, Compound 1 Glutarate has a characteristic XRPD peak in terms of 2θ at 18.2°±0.2°.

In some embodiments, Compound 1 Glutarate has characteristic XRPD peaks in terms of 2θ selected from 9.1°±0.2°, 10.6°±0.2°, 18.2°±0.2°, and 19.0°±0.2°. In some embodiments, Compound 1 Glutarate has at least one characteristic XRPD peak in terms of 2θ selected from 9.1°±0.2°, 10.6°±0.2°, 18.2°±0.2°, and 19.0°±0.2°.

In some embodiments, Compound 1 Glutarate has at least one characteristic XRPD peak in terms of 2θ selected from 9.1°±0.2°, 10.6°±0.2°, 18.2°±0.2°, 19.0°±0.2°, 22.5°±0.2°, 27.4°±0.2°, and 28.0°±0.2°. In some embodiments, Compound 1 Glutarate has at least one characteristic XRPD peak in terms of 2θ selected from 9.1°±0.2°, 10.6°±0.2°, 18.2°±0.2°, 19.0°±0.2°, 21.8°±0.2°, 21.9°±0.2°, 22.5°±0.2°, 25.8°±0.2°, 27.4°±0.2°, and 28.0°±0.2°.

In some embodiments, Compound 1 Glutarate has at least two characteristic XRPD peaks in terms of 2θ selected from 9.1°±0.2°, 10.6°±0.2°, 18.2°±0.2°, 19.0°±0.2°, 22.5°±0.2°, 27.4°±0.2°, and 28.0°±0.2°. In some embodiments, Compound 1 Glutarate has at least two characteristic XRPD peaks in terms of 2θ selected from 9.1°±0.2°, 10.6°±0.2°, 18.2°±0.2°, 19.0°±0.2°, 21.8°±0.2°, 21.9°±0.2°, 22.5°±0.2°, 25.8°±0.2°, 27.4°±0.2°, and 28.0°±0.2°.

In some embodiments, Compound 1 Glutarate has at least three characteristic XRPD peaks in terms of 2θ selected from 9.1°±0.2°, 10.6°±0.2°, 18.2°±0.2°, 19.0°±0.2°, 22.5°±0.2°, 27.4°±0.2°, and 28.0°±0.2°. In some embodiments, Compound 1 Glutarate has at least three characteristic XRPD peaks in terms of 2θ selected from 9.1°±0.2°, 10.6°±0.2°, 18.2°±0.2°, 19.0°±0.2°, 21.8°±0.2°, 21.9°±0.2°, 22.5°±0.2°, 25.8°±0.2°, 27.4°±0.2°, and 28.0°±0.2°.

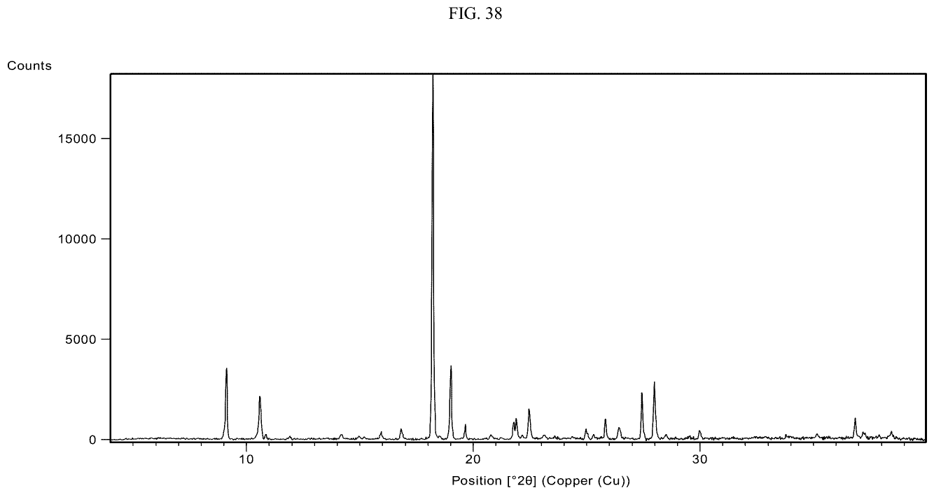

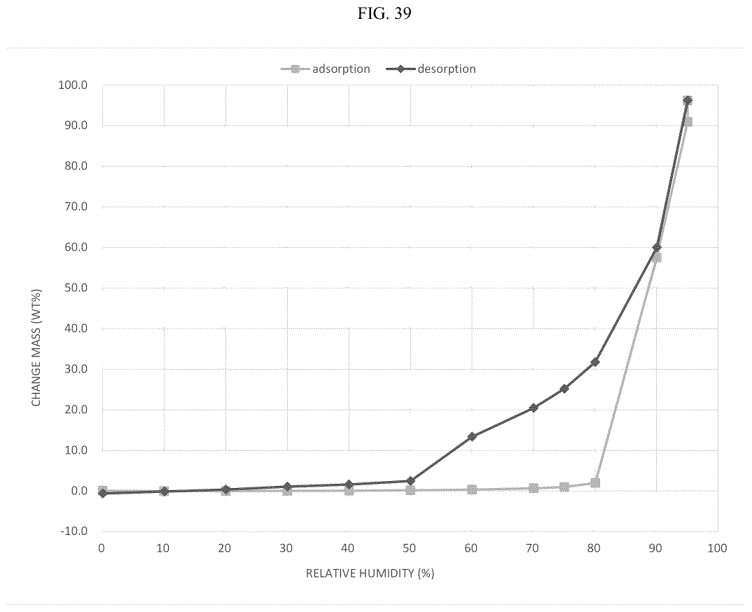

In some embodiments, Compound 1 Glutarate has an XRPD pattern with characteristic peaks as substantially shown in FIG. 38 (FIG. 38). In some embodiments, Compound 1 Glutarate has a DVS isotherm as substantially shown in FIG. 39 (FIG. 39).

In some embodiments, Compound 1 Glutarate can be isolated with a crystalline purity of at least about 80%, about 85%, about 90%, about 95%, about 96%, about 97%, about 98%, or about 99%. In some embodiments, Compound 1 Glutarate can be isolated with a crystalline purity greater than about 99%. In some embodiments, Compound 1 Glutarate can be isolated with a crystalline purity greater than about 99.9%.

In some embodiments, provided is Compound 1 Glutarate prepared by isolating Compound 1 glutarate from a mixture of Compound 1, glutaric acid, and S1, wherein S1 is a solvent. In some embodiments, S1 is an organic solvent. In some embodiments, S1 is C₁₋₆ alkyl alcohol. In some embodiments, S1 is C₁₋₆ alkyl acetate. In some embodiments, S1 is a C₁₋₆ alkyl ketone. In some embodiments, S1 is a mixture of organic solvents. In some embodiments, S1 is a mixture of a C₁₋₆ alkyl alcohol and a C₁₋₆ alkyl ketone. In some embodiments, S1 is a mixture of methanol and acetone. In some embodiments, S1 is THF. In some embodiments, S1 is ethyl acetate.

### Compound 1 L-Malate

In some embodiments, provided is (R)-1-(8-fluoroisochroman-1-yl)-N-methylmethanamine L-Malate. In some embodiments, Compound 1 L-Malate is crystalline.

In some embodiments, Compound 1 L-Malate has characteristic XRPD peaks in terms of 2θ selected from 13.5°±0.2°, 18.8°±0.2°, and 25.2°±0.2°. In some embodiments, Compound 1 L-Malate has a characteristic XRPD peak in terms of 2θ at 13.5°±0.2°. In some embodiments, Compound 1 L-Malate has a characteristic XRPD peak in terms of 2θ at 18.8° 0.2°. In some embodiments, Compound 1 L-Malate has a characteristic XRPD peak in terms of 2θ at 25.2°±0.2°.

In some embodiments, Compound 1 L-Malate has at least one characteristic XRPD peak in terms of 2θ selected from 13.5°±0.2°, 14.4°±0.2°, 15.2°±0.2°, 18.8°±0.2°, 23.8°±0.2°, 24.8°±0.2°, and 25.2°±0.2°. In some embodiments, Compound 1 L-Malate has at least one characteristic XRPD peak in terms of 2θ selected from 13.5°±0.2°, 14.4°±0.2°, 15.2°±0.2°, 17.7°±0.2°, 18.8°±0.2°, 22.4°±0.2°, 23.8°±0.2°, 24.6°±0.2°, 24.8°±0.2°, and 25.2°±0.2°.

In some embodiments, Compound 1 L-Malate has at least two characteristic XRPD peaks in terms of 2θ selected from 13.5°±0.2°, 14.4°±0.2°, 15.2°±0.2°, 18.8°±0.2°, 23.8°±0.2°, 24.8°±0.2°, and 25.2°±0.2°. In some embodiments, Compound 1 L-Malate has at least two characteristic XRPD peaks in terms of 2θ selected from 13.5°±0.2°, 14.4°±0.2°, 15.2°±0.2°, 17.7°±0.2°, 18.8°±0.2°, 22.4°±0.2°, 23.8°±0.2°, 24.6°±0.2°, 24.8°±0.2°, and 25.2°±0.2°.

In some embodiments, Compound 1 L-Malate has at least three characteristic XRPD peaks in terms of 2θ selected from 13.5°±0.2°, 14.4°±0.2°, 15.2°±0.2°, 18.8°±0.2°, 23.8°±0.2°, 24.8°±0.2°, and 25.2°±0.2°. In some embodiments, Compound 1 L-Malate has at least three characteristic XRPD peaks in terms of 2θ selected from 13.5°±0.2°, 14.4°±0.2°, 15.2°±0.2°, 17.7°±0.2°, 18.8°±0.2°, 22.4°±0.2°, 23.8°±0.2°, 24.6°±0.2°, 24.8°±0.2°, and 25.2°±0.2°.

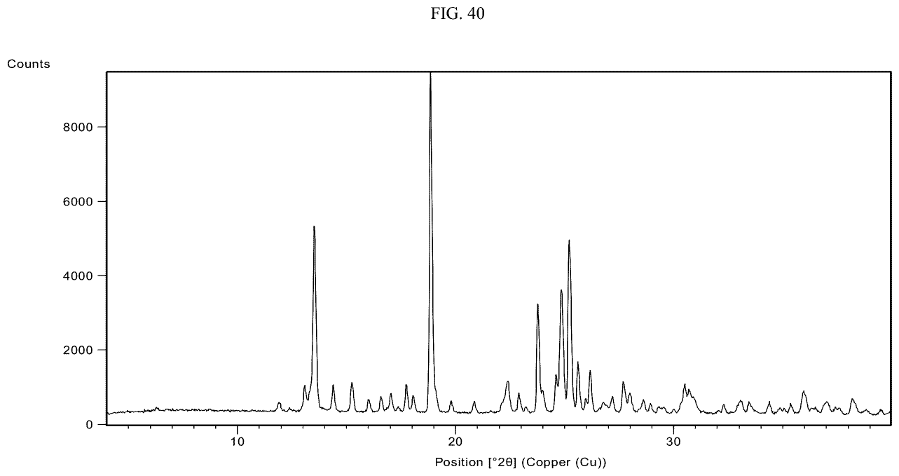

In some embodiments, Compound 1 L-Malate has an XRPD pattern with characteristic peaks as substantially shown in FIG. 40 (FIG. 40).

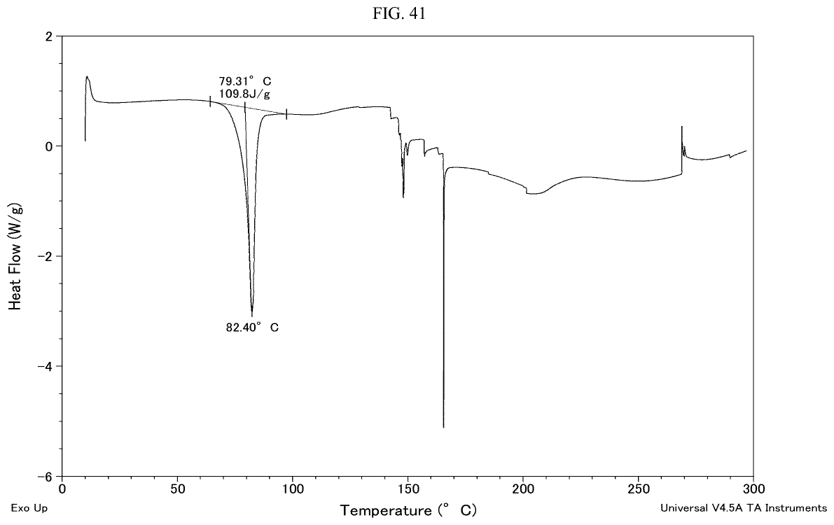

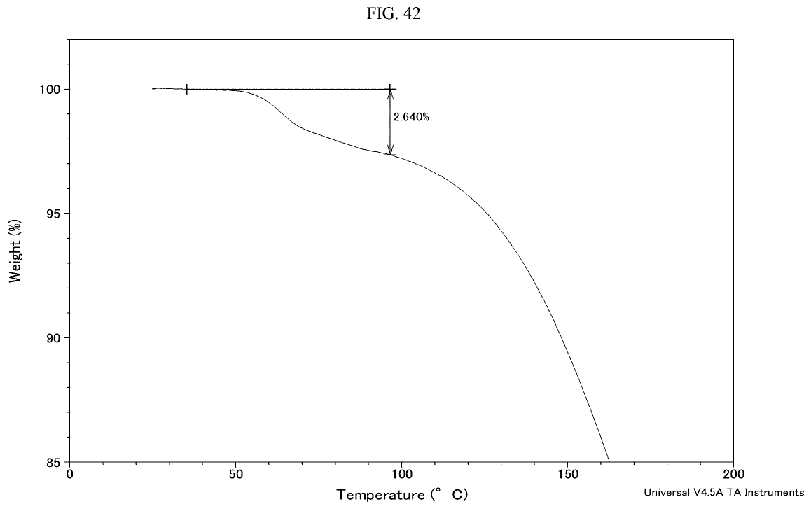

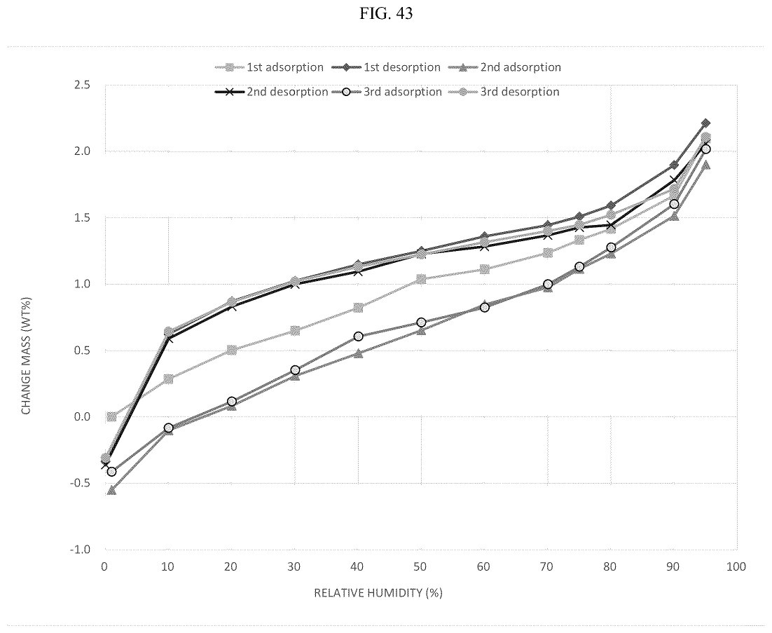

In some embodiments, Compound 1 L-Malate has an endotherm peak at a temperature of about 82° C. In some embodiments, Compound 1 L-Malate has a DSC thermogram substantially as depicted in FIG. 41 (FIG. 41). In some embodiments, Compound 1 L-L-Malate has a TGA thermogram substantially as depicted in FIG. 42 (FIG. 42). In some embodiments, Compound 1 L-Malate has a DVS isotherm substantially as depicted in FIG. 43 (FIG. 43).

In some embodiments, Compound 1 L-Malate has at least one characteristic XRPD peak in terms of 2θ selected from 13.5°±0.2°, 18.8°±0.2°, and 25.2°±0.2°; and an endotherm peak at a temperature of about 82° C. In some embodiments, Compound 1 L-Malate has at least one characteristic XRPD peak in terms of 2θ selected from 13.5°±0.2°, 18.8°±0.2°, and 25.2°±0.2°; and a DSC thermogram substantially as depicted in FIG. 41 (FIG. 41). In some embodiments, Compound 1 L-Malate has at least one characteristic XRPD peak in terms of 2θ selected from 13.5°±0.2°, 18.8°±0.2°, and 25.2°±0.2°; and a DVS isotherm substantially as depicted in FIG. 43 (FIG. 43).

In some embodiments, Compound 1 L-Malate can be isolated with a crystalline purity of at least about 80%, about 85%, about 90%, about 95%, about 96%, about 97%, about 98%, or about 99%. In some embodiments, Compound 1 L-Malate can be isolated with a crystalline purity greater than about 99%. In some embodiments, Compound 1 L-Malate can be isolated with a crystalline purity greater than about 99.9%.

In some embodiments, provided is Compound 1 L-Malate prepared by isolating Compound 1 L-malate from a mixture of Compound 1, L-malic acid, and S1, wherein S1 is a solvent. In some embodiments, S1 is an organic solvent. In some embodiments, S1 is C₁₋₆ alkyl alcohol. In some embodiments, S1 is ether. In some embodiments, S1 is C₁₋₆ alkyl acetate. In some embodiments, S1 is methanol. In some embodiments, S1 is THF. In some embodiments, S1 is ethyl acetate.

### Compound 1 Besylate

In some embodiments, provided is (R)-1-(8-fluoroisochroman-1-yl)-N-methylmethanamine Besylate. In some embodiments, Compound 1 Besylate is crystalline. In some embodiments, Compound 1 Besylate has characteristic XRPD peaks in terms of 2θ selected from 6.0°±0.2°, 12.0°±0.2°, and 24.1°±0.2°. In some embodiments, Compound 1 Besylate has a characteristic XRPD peak in terms of 2θ at 6.0°±0.2°. In some embodiments, Compound 1 Besylate has a characteristic XRPD peak in terms of 2θ at 12.0°±0.2°. In some embodiments, Compound 1 Besylate has a characteristic XRPD peak in terms of 2θ at 24.1 0.2°.

In some embodiments, Compound 1 Besylate has at least one characteristic XRPD peak in terms of 2θ selected from 6.0°±0.2°, 12.0°±0.2°, 16.6°±0.2°, 24.1°±0.2°, 26.8°±0.2°, and 30.3°±0.2°. In some embodiments, Compound 1 Besylate has at least one characteristic XRPD peak in terms of 2θ selected from 6.0°±0.2°, 12.0°±0.2°, 16.4°±0.2°, 16.6°±0.2°, 19.0°±0.2°, 21.2°±0.2°, 22.2°±0.2°, 23.2°±0.2°, 24.1°±0.2°, 26.8°±0.2°, and 30.3°±0.2°.

In some embodiments, Compound 1 Besylate has at least two characteristic XRPD peaks in terms of 2θ selected from 6.0°±0.2°, 12.0°±0.2°, 16.6°±0.2°, 24.1°±0.2°, 26.8°±0.2°, and 30.3°±0.2°. In some embodiments, Compound 1 Besylate has at least two characteristic XRPD peaks in terms of 2θ selected from 6.0°±0.2°, 12.0°±0.2°, 16.4°±0.2°, 16.6°±0.2°, 19.0°±0.2°, 21.2°±0.2°, 22.2°±0.2°, 23.2°±0.2°, 24.1°±0.2°, 26.8°±0.2°, and 30.3°±0.2°.

In some embodiments, Compound 1 Besylate has at least three characteristic XRPD peaks in terms of 2θ selected from 6.0°±0.2°, 12.0°±0.2°, 16.6°±0.2°, 24.1°±0.2°, 26.8°±0.2°, and 30.3°±0.2°. In some embodiments, Compound 1 Besylate has at least three characteristic XRPD peaks in terms of 2θ selected from 6.0°±0.2°, 12.0°±0.2°, 16.4°±0.2°, 16.6°±0.2°, 19.0°±0.2°, 21.2°±0.2°, 22.2°±0.2°, 23.2°±0.2°, 24.1°±0.2°, 26.8°±0.2°, and 30.3°±0.2°.

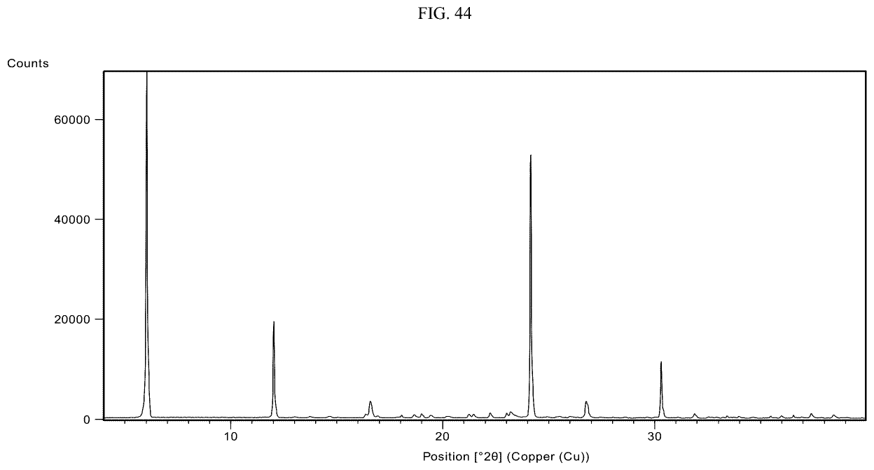

In some embodiments, Compound 1 Besylate has an XRPD pattern with characteristic peaks as substantially shown in FIG. 44 (FIG. 44).

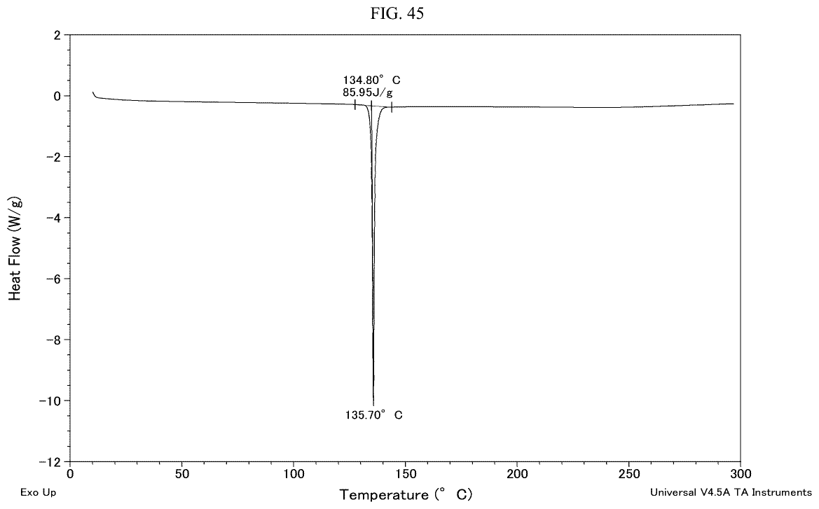

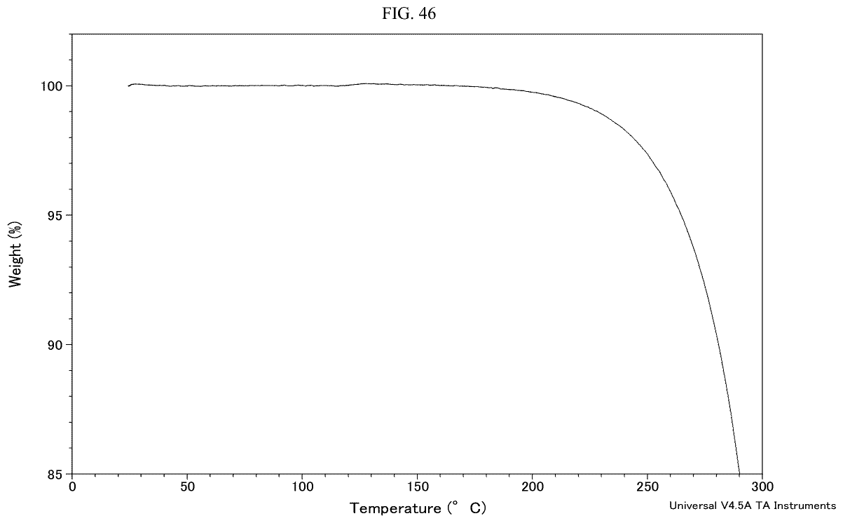

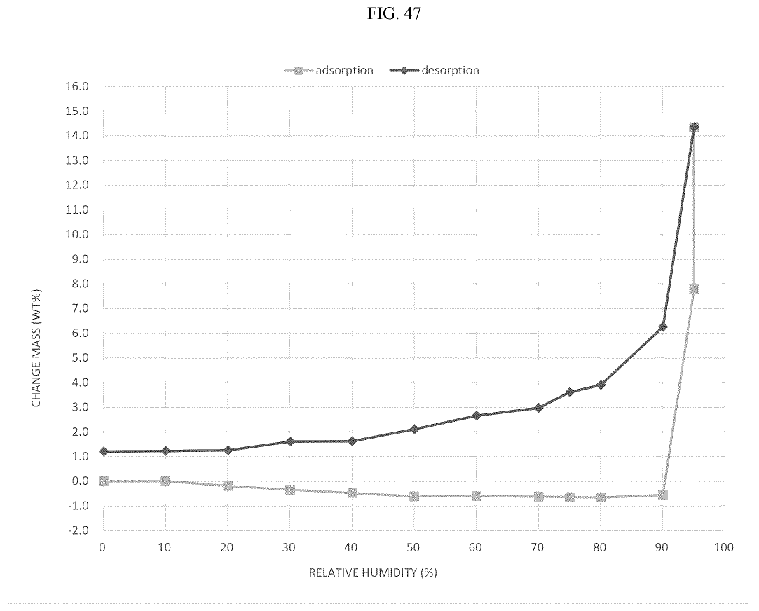

In some embodiments, Compound 1 Besylate has an endotherm peak at a temperature of about 136° C. In some embodiments, Compound 1 Besylate has a DSC thermogram substantially as depicted in FIG. 45 (FIG. 45). In some embodiments, Compound 1 Besylate has a TGA thermogram substantially as depicted in FIG. 46 (FIG. 46). In some embodiments, Compound 1 Besylate has a DVS isotherm substantially as depicted in FIG. 47 (FIG. 47).

In some embodiments, Compound 1 Besylate has at least one characteristic XRPD peak in terms of 2θ selected from 6.0°±0.2°, 12.0°±0.2°, and 24.1°±0.2°; and an endotherm peak at a temperature of about 136° C. In some embodiments, Compound 1 Besylate has at least one characteristic XRPD peak in terms of 2θ selected from 6.0°±0.2°, 12.0°±0.2°, and 24.1°±0.2°; and a DSC thermogram substantially as depicted in FIG. 45 (FIG. 45). In some embodiments, Compound 1 Besylate has at least one characteristic XRPD peak in terms of 2θ selected from 6.0°±0.2°, 12.0°±0.2°, and 24.1°±0.2°; and a DVS isotherm substantially as depicted in FIG. 47 (FIG. 47).

In some embodiments, Compound 1 Besylate can be isolated with a crystalline purity of at least about 80%, about 85%, about 90%, about 95%, about 96%, about 97%, about 98%, or about 99%. In some embodiments, Compound 1 Besylate can be isolated with a crystalline purity greater than about 99%. In some embodiments, Compound 1 Besylate can be isolated with a crystalline purity greater than about 99.9%.

In some embodiments, provided is Compound 1 Besylate prepared by isolating Compound 1 Besylate from a mixture of Compound 1, benzenesulfonic acid, and S1, wherein S1 is a solvent. In some embodiments, S1 is an organic solvent. In some embodiments, S1 is C₁₋₆ alkyl alcohol. In some embodiments, S1 is ether. In some embodiments, S1 is C₁₋₆ alkyl acetate. In some embodiments, S1 is a C₁₋₆ alkyl ketone. In some embodiments, S1 is a mixture of organic solvents. In some embodiments, S1 is a mixture of a C₁₋₆ alkyl alcohol and a C₁₋₆ alkyl ketone. In some embodiments, S1 is ethyl acetate. In some embodiments, S1 is THF. In some embodiments, S1 is a mixture of methanol and acetone.

### Compound 1 Tosylate

In some embodiments, provided is (R)-1-(8-fluoroisochroman-1-yl)-N-methylmethanamine tosylate (Compound 1 Tosylate).

In some embodiments, provided is Compound 1 Tosylate prepared by isolating Compound 1 Tosylate from a mixture of Compound 1, p-toluenesulfonic acid, and S1, wherein S1 is a solvent. In some embodiments, S1 is an organic solvent. In some embodiments, S1 is C₁₋₆ alkyl alcohol. In some embodiments, S1 is ether. In some embodiments, S1 is C₁₋₆ alkyl acetate. In some embodiments, S1 is a C₁₋₆ alkyl ketone. In some embodiments, S1 is a mixture of organic solvents. In some embodiments, S1 is a mixture of a C₁₋₆ alkyl alcohol and a C₁₋₆ alkyl ketone. In some embodiments, S1 is methanol. In some embodiments, S1 is THF. In some embodiments, S1 is ethyl acetate. In some embodiments, S1 is a mixture of methanol and acetone.

### Process for Preparation of Compound 1 and Compound 1 Phosphate

Provided herein are also processes for preparing Compound 1 or a salt thereof. A process of preparing Compound 1 is described in U.S. Pat. No. 10,196,403, the entirety of which is incorporated herein by reference. The processes for preparing Compound 1 or a salt thereof provided herein have certain advantages over the processes currently disclosed in the art. For example, the processes described herein demonstrate good scalability, yields, and stereochemical selectivity. The processes described herein include a chiral resolution by crystallization, which avoids chiral separation by HPLC, and is therefore more amenable for manufacture on a multi-kilogram scale.

Provided herein is a process of preparing (R)-1-(8-fluoroisochroman-1-yl)-N-methylmethanamine phosphate (Compound 1 Phosphate), having the structure:

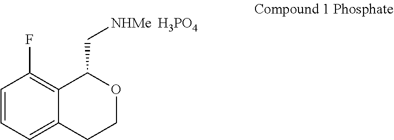

wherein the process comprises:
(a) reacting a compound of Formula III having the structure:

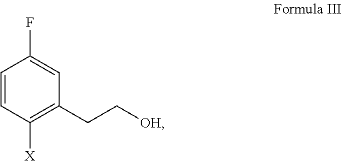

or a salt thereof, wherein X is halo, with N-methylaminoacetaldehyde dimethylacetal (Compound 4) having the structure:

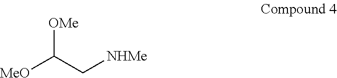

in the presence of A1, wherein A1 is an acid, to provide a compound of Formula II, having the structure:

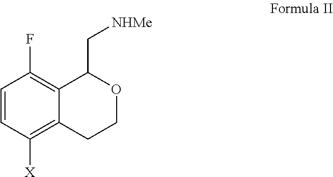

or a salt thereof, wherein X is halo;
(b) hydrogenating a compound of Formula II, or a salt thereof, in the presence of a metal catalyst to provide 1-(8-fluoroisochroman-1-yl)-N-methylmethanamine (Racemic Compound 1) having the structure:

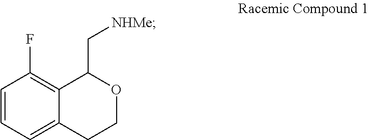 [SMILES: CNCC1OCCc2cccc(F)c21]

(c) reacting Racemic Compound 1 with dibenzoyl-L-tartaric acid in the presence of S3, wherein S3 is a solvent, to provide (R)-1-(8-fluoroisochroman-1-yl)-N-methylmethanamine dibenzoyl-L-tartrate (Compound 1 dibenzoyl-L-tartrate) having the structure:

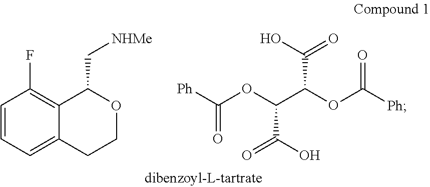 [SMILES: CNC[C@@H]1OCCc2cccc(F)c21 ; O=C(O[C@@H](C(=O)O)[C@@H](OC(=O)c1ccccc1)C(=O)O)c1ccccc1]

(d) reacting Compound 1 dibenzoyl-L-tartrate with B1, wherein B1 is a base, to provide Compound 1 having the structure:

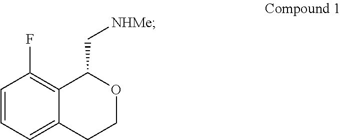 [SMILES: CNC[C@@H]1OCCc2cccc(F)c21]

and
(e) reacting Compound 1 with phosphoric acid to provide Compound 1 Phosphate.

Provided herein is a process of preparing Compound 1 having the structure:

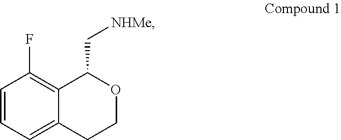 [SMILES: CNC[C@@H]1OCCc2cccc(F)c21]

wherein the process comprises:
(a) reacting a compound of Formula III having the structure:

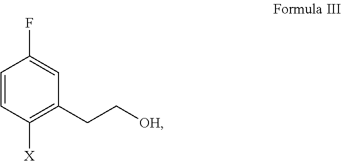

or a salt thereof, wherein X is halo, with N-methylaminoacetaldehyde dimethylacetal (Compound 4) having the structure:

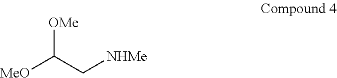

in the presence of A1, wherein A1 is an acid, to provide a compound of Formula II, having the structure:

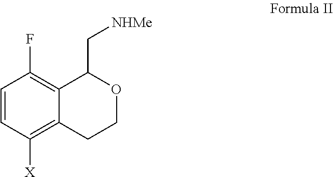

or a salt thereof, wherein X is halo;
(b) hydrogenating a compound of Formula II, or a salt thereof, in the presence of a metal catalyst to provide 1-(8-fluoroisochroman-1-yl)-N-methylmethanamine (Racemic Compound 1) having the structure:

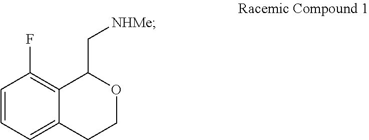 [SMILES: CNCC1OCCc2cccc(F)c21]

(c) reacting Racemic Compound 1 with dibenzoyl-L-tartaric acid in the presence of S3, wherein S3 is a solvent, to provide (R)-1-(8-fluoroisochroman-1-yl)-N-methylmethanamine dibenzoyl-L-tartrate (Compound 1 dibenzoyl-L-tartrate) having the structure:

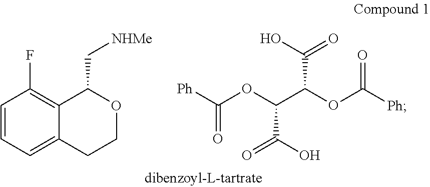 [SMILES: CNC[C@@H]1OCCc2cccc(F)c21 ; O=C(O[C@@H](C(=O)O)[C@@H](OC(=O)c1ccccc1)C(=O)O)c1ccccc1]

Compound 1 dibenzoyl-L-tartrate; and
(d) reacting Compound 1 dibenzoyl-L-tartrate with B1, wherein B1 is a base, to provide Compound 1.

In some embodiments, provided is a process of preparing Compound 1, wherein the process comprises:
(a) hydrogenating a compound of Formula II, or a salt thereof, in the presence of a metal catalyst to provide Racemic Compound 1;
(b) reacting Racemic Compound 1 with dibenzoyl-L-tartaric acid in the presence of S3, wherein S3 is a solvent, to provide Compound 1 dibenzoyl-L-tartrate; and
(c) reacting Compound 1 dibenzoyl-L-tartrate with B1, wherein B1 is a base, to provide Compound 1.

In some embodiments, provided is a process of preparing Compound 1, wherein the process comprises:
(a) reacting Racemic Compound 1 with dibenzoyl-L-tartaric acid in the presence of S3, wherein S3 is a solvent, to provide Compound 1 dibenzoyl-L-tartrate; and
(c) reacting Compound 1 dibenzoyl-L-tartrate with B1, wherein B1 is a base, to provide Compound 1.

Provided herein is a process of preparing (R)-1-(8-fluoroisochroman-1-yl)-N-methylmethanamine phosphate (Compound 1 Phosphate), having the structure:

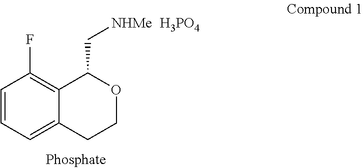

comprising reacting (R)-1-(8-fluoroisochroman-1-yl)-N-methylmethanamine (Compound 1) having the structure:

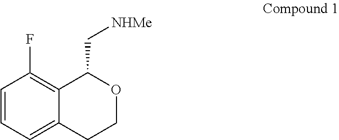 [SMILES: CNC[C@@H]1OCCc2cccc(F)c21]

with phosphoric acid. In some embodiments, Compound 1 phosphate is crystalline.

In some embodiments, the phosphoric acid is an aqueous solution of phosphoric acid. In some embodiments, the aqueous solution of phosphoric acid is about 80% to about 95% aqueous solution of phosphoric acid by weight. In some embodiments, the aqueous solution of phosphoric acid is about 87% aqueous solution of phosphoric acid by weight.

In some embodiments, the reacting of Compound 1 with phosphoric acid is carried out in the presence of Sla, wherein Sla is a solvent. In some embodiments, Sla is a polar aprotic solvent, water, or a mixture thereof. In some embodiments, the polar aprotic solvent of S1 is acetonitrile. In some embodiments, S1a is a mixture of acetonitrile and water.

In some embodiments, the reacting of Compound 1 with phosphoric acid is carried out at a temperature between about 15° C. and about 25° C. In some embodiments, the reacting of Compound 1 with phosphoric acid is carried out at about 20° C. In some embodiments, between about 1 and about 5 molar equivalents of phosphoric acid are used per molar equivalent of Compound 1. In some embodiments, about 1 molar equivalent of phosphoric acid is used per molar equivalent of Compound 1.

Compound 1 can be prepared by a process comprising reacting (R)-1-(8-fluoroisochroman-1-yl)-N-methylmethanamine dibenzoyl-L-tartrate (Compound 1 dibenzoyl-L-tartrate) having the structure:

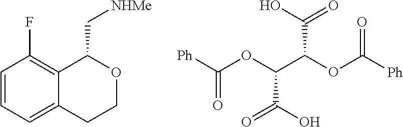 [SMILES: CNC[C@@H]1OCCc2cccc(F)c21 ; O=C(O[C@@H](C(=O)O)[C@@H](OC(=O)c1ccccc1)C(=O)O)c1ccccc1]

Compound 1 dibenzoyl-L-tartrate with B1, wherein B1 is a base.

In some embodiments, B1 is an alkali hydroxide base. In some embodiments, B1 is potassium hydroxide. In some embodiments, B1 is an aqueous solution of potassium hydroxide. In some embodiments, the aqueous solution of potassium hydroxide is about 10% to about 20% aqueous solution of potassium hydroxide by weight. In some embodiments, the aqueous solution of potassium hydroxide is about 14% aqueous solution of potassium hydroxide by weight.

In some embodiments, the reacting of Compound 1 dibenzoyl-L-tartrate and the base is carried out in the presence of S2, wherein S2 is a solvent. In some embodiments, S2 is a polar aprotic solvent. In some embodiments, the polar aprotic solvent is an ether. In some embodiments, S2 is tert-butyl methyl ether.

In some embodiments, the reacting of Compound 1 dibenzoyl-L-tartrate and B1 is carried out at a temperature between about 20° C. and about 30° C. In some embodiments, the reacting of Compound 1 dibenzoyl-L-tartrate and the base is carried out at a temperature of about 23° C. In some embodiments, between about 0.5 and about 5 molar equivalents of B1 are used per molar equivalent of Compound 1 dibenzoyl-L-tartrate. In some embodiments, between about 1 and about 3 molar equivalents of B1 are used per molar equivalent of Compound 1 dibenzoyl-L-tartrate. In some embodiments, between about 1 and about 2 molar equivalents of B1 are used per molar equivalent of Compound 1 dibenzoyl-L-tartrate. In some embodiments, about 1 molar equivalent of B1 is used per molar equivalent of Compound 1 dibenzoyl-L-tartrate.

In some embodiments, the reacting of Compound 1 dibenzoyl-L-tartrate with B1 is further carried out in the presence of sodium chloride. In some embodiments, about 1 to about 10 molar equivalents of sodium chloride are used per molar equivalent of Compound 1 dibenzoyl-L-tartrate. In some embodiments, about 5 molar equivalents of sodium chloride are used per molar equivalent of Compound 1 dibenzoyl-L-tartrate.

(R)-1-(8-fluoroisochroman-1-yl)-N-methylmethanamine dibenzoyl-L-tartrate (Compound 1 dibenzoyl-L-tartrate) can be prepared by a process comprising reacting 1-(8-fluoroisochroman-1-yl)-N-methylmethanamine (Racemic Compound 1) having the structure:

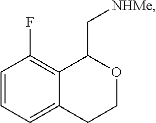 [SMILES: CNCC1OCCc2cccc(F)c21]

with dibenzoyl-L-tartaric acid, in the presence of S3, wherein S3 is a solvent.

In some embodiments, S3 is a polar protic solvent. In some embodiments, S3 is C₁₋₆ alkyl-OH. In some embodiments, S3 is methanol. In some embodiments, S3 is a mixture of methanol and water. In some embodiments, the reacting is carried out at a temperature from about 20° C. to about 70° C. In some embodiments, the precipitating is carried out at a temperature of about 20° C. In some embodiments, about 1 to about 5 molar equivalents of dibenzoyl-L-tartaric acid are used per molar equivalent of Racemic Compound 1. In some embodiments, about 1 molar equivalent of dibenzoyl-L-tartaric acid is used per molar equivalent of Racemic Compound 1.

The process of preparing Compound 1 dibenzoyl L-tartrate can further comprise precipitating Compound 1 dibenzoyl-L-tartrate from a mixture comprising: Racemic Compound 1, dibenzoyl-L-tartaric acid, and S3. In some embodiments, S3 is methanol. In some embodiments, S3 is a mixture of methanol and water.

The process of preparing Compound 1 dibenzoyl L-tartrate can further comprise isolating Compound 1 dibenzoyl-L-tartrate from S3a, wherein S3a is a solvent. In some embodiments, S3a is polar protic solvent. In some embodiments, S3a is C₁₋₆ alkyl-OH. In some embodiments, S3a is methanol. In some embodiments, the isolating of Compound 1 dibenzoyl-L-tartrate is carried out at a temperature of about 10° C. to about 65° C.

1-(8-fluoroisochroman-1-yl)-N-methylmethanamine (Racemic Compound 1) can be prepared by a process comprising hydrogenating a compound of Formula II, having the structure:

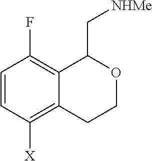

or a salt thereof, wherein X is halo, in the presence of a metal catalyst.

In some embodiments, the compound of Formula II is Compound 2 is 1-(5-bromo-8-fluoroisochroman-1-yl)-N-methylmethanamine trifluoromethanesulfonate (Compound 2) having the structure:

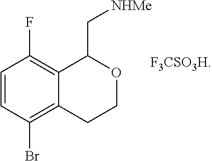 [SMILES: CNCC1OCCc2c(Br)ccc(F)c21]

In some embodiments, the metal catalyst is palladium on activated carbon. In some embodiments, the hydrogenating of a compound of Formula II is carried out at a hydrogen pressure of about 2 to about 10 bar. In some embodiments, the hydrogenating of a compound of Formula II is carried out at a hydrogen pressure of about 5 bar.

In some embodiments, the hydrogenating of a compound of Formula II is carried out at a temperature between about 20° C. and about 30° C. In some embodiments, the hydrogenating of a compound of Formula II is carried out at a temperature of about 25° C.

In some embodiments, the hydrogenating of a compound of Formula II is carried out in the presence of S4, wherein S4 is a solvent. In some embodiments, S4 is a polar protic solvent, In some embodiments, S4 is C₁₋₆ alkyl-OH. In some embodiments, S4 is methanol.

In some embodiments, the hydrogenating of a compound of Formula II is carried out in the presence of B2, wherein B2 is a base. In some embodiments, B2 is a carbonate base. In some embodiments, B2 is potassium carbonate. In some embodiments, B2 is an aqueous solution of potassium carbonate. In some embodiments, the aqueous solution of potassium carbonate is about 5% potassium carbonate by weight.

The compound of Formula II can be prepared by reacting a compound of Formula III having the structure:

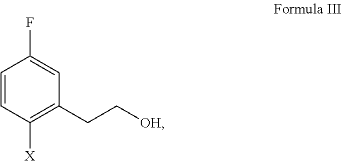

or a salt thereof, wherein X is halo,
with N-methylaminoacetaldehyde dimethylacetal (Compound 4) having the structure:

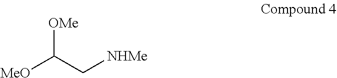

in the presence of A1, wherein A1 is an acid.

In some embodiments, the compound of Formula III is 2-(2-bromo-5-fluorophenyl)ethan-1-ol (Compound 3) having the structure:

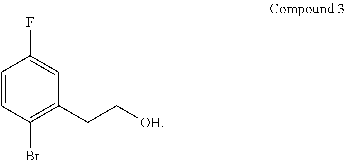 [SMILES: OCCc1cc(F)ccc1Br]

In some embodiments, A1 is trifluoromethanesulfonic acid. In some embodiments, the reacting of the compound of Formula III and Compound 4 is carried out in the presence of S5, wherein S5 is a solvent. In some embodiments, S5 is a halogenated solvent. In some embodiments, S5 is dichloromethane.

In some embodiments, the reacting of the compound of Formula III and Compound 4 is carried out a temperature between about 0° C. and about 35° C. In some embodiments, the reacting of the compound of Formula III and Compound 4 is carried out a temperature of about 30° C. In some embodiments, about 1.2 molar equivalents of Compound 4 are used per molar equivalent of the compound of Formula III. In some embodiments, about 4 molar equivalents of A1 are used per molar equivalent of the compound of Formula III.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

The reactions of the processes described herein can be carried out in air or under an inert atmosphere. Typically, reactions containing reagents or products that are substantially reactive with air can be carried out using air-sensitive synthetic techniques that are well known to the skilled artisan.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as high performance liquid chromatography (HPLC) or thin layer chromatography. The compounds obtained by the reactions can be purified by any suitable method known in the art. For example, chromatography (medium pressure) on a suitable adsorbent (e.g., silica gel, alumina and the like), HPLC, or preparative thin layer chromatography; distillation; sublimation, trituration, or recrystallization. The purity of the compounds, in general, are determined by physical methods such as measuring the melting point (in case of a solid), obtaining a NMR spectrum, or performing a HPLC separation. If the melting point decreases, if unwanted signals in the NMR spectrum are decreased, or if extraneous peaks in an HPLC trace are removed, the compound can be said to have been purified. In some embodiments, the compounds are substantially purified.

### Method of Use

In some embodiments, the disclosure provides a method for treating a neurological or psychiatric disease or disorder in a subject, comprising administering to the subject an effective amount of a compound of this disclosure (or its pharmaceutically acceptable salt), or composition comprising a compound of this disclosure (or its pharmaceutically acceptable salt). Neurological and/or psychiatric diseases and disorders can exhibit a variety of psychiatric and behavioral symptoms, including apathy, depression, anxiety, cognitive impairment, psychosis, aggression, agitation, poor impulse control and sleep disruptions.

In one embodiment, the neurological or psychiatric disease or disorder is bipolar disorder, anxiety, depression, Alzheimer's disease with agitation, Alzheimer's disease with aggression, Alzheimer's disease agitation or Alzheimer's disease with agitation aggression.

In one embodiment, the neurological or psychiatric disease or disorder is bipolar disorder, anxiety, depression, dementia, Alzheimer's disease, Alzheimer's disease with agitation, Alzheimer's disease with aggression, Alzheimer's disease agitation or Alzheimer's disease with agitation aggression, a neurocognitive disorder, a neurocognitive disorder with behavioral and psychological symptoms.

In one embodiment, the neurological or psychiatric disease or disorder are behavioral and psychological symptoms of a neurocognitive disorder including dementia and Alzheimer's disease. The behavioral and psychological symptoms include disturbances in perception, thought content, mood, or behaviors including delusions (distressing beliefs), hallucinations, psychoses, agitation (easily upset, repeating questions, arguing or complaining, hoarding, pacing, inappropriate screaming, crying out, disruptive sounds, rejection of care leaving home), aggression (physical or verbal), depression or dysphoria, anxiety (worrying, shadowing), apathy or indifference, disinhibition (socially inappropriate behavior, sexually inappropriate behavior, irritability or lability, motor disturbance (repetitive activities without purpose, wandering, rummaging, night-time behaviors (waking and getting up at night) impulsivity, attentional deficits, executive dysfunction.

Assays were used herein to identify representative candidate treatments. Examples of candidate treatments include, without limitation, treatment of Alzheimer's disease with agitation, Alzheimer's disease with aggression, Alzheimer's disease agitation and Alzheimer's disease with agitation aggression. Aggression and agitation are common symptoms in neurological and psychiatric diseases and disorders. Aggression and agitation have been associated with hyperactivity in subcortical brain regions, which can be modelled in animals using psychostimulants (e.g., PCP, Amphetamine). For example, psychostimulants induce hyperlocomotor activity (HLA) in animals. Antipsychotics (e.g., haloperidol, clozapine and risperidone) have been shown to reduce psychostimulant-induced HLA and are efficacious against agitation in Alzheimer's disease. Other drugs used off-label, or are currently under study in clinical trials, for agitation in Alzheimer's disease are mood stabilizers, such as lithium (which also decreases Amphetamine-induced HLA), and antidepressants (e.g., citalopram). Antidepressants demonstrate activity in assays such as the forced swim and tail suspension tests. Therefore, the aforementioned assays were helpful in identifying candidate treatments for agitation in Alzheimer's disease and agitation/aggression in other neurological and psychiatric diseases and disorders.

In one embodiment, provided is a method of treating a bipolar disorder in a subject in need thereof, comprising the step of administering to said subject an effective amount of the compound according to formula I or a pharmaceutically acceptable salt thereof.

In one embodiment, provided is a method of treating anxiety in a subject in need thereof, comprising the step of administering to said subject an effective amount of the compound according to formula I or a pharmaceutically acceptable salt thereof.

In some embodiments, the neurological or psychiatric disease or disorder is selected from a psychosis, including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (e.g., phencyclidine, ketamine and other dissociative anesthetics, amphetamine and other psychostimulants and cocaine) psychosis, psychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both positive, negative, and cognitive symptoms of schizophrenia and other psychoses; cognitive disorders including dementia (semantic dementia, frontotemporal dementia, dementia with depressive features, persisting, subcortical dementia, dementia with Lewy Bodies, Parkinsonism-ALS Dementia Complex, and dementia associated with Alzheimer's disease, ischemia, multi-infarct dementia, trauma, vascular problems, stroke, HIV disease, Parkinson's disease, Huntington's disease, Down syndrome, Pick's disease, Creutzfeldt-Jacob disease, perinatal hypoxia, or substance abuse), delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); eating disorders such as obesity, bulimia nervosa, pica and compulsive eating disorders; bipolar disorders, including bipolar I disorder, bipolar II disorder, cyclothymic disorder, substance/medication-induced bipolar and related disorders, bipolar and related disorder due to another medical condition, other specified bipolar and related disorder, and unspecified bipolar and related disorders, depressive disorders including unipolar depression, seasonal depression and post-partum depression, atypical depression, catatonic depression, elderly depression, endogenous depression, melancholic depression, perinatal depression, situational depression, chronic depression, premenstrual syndrome (PMS) and premenstrual dysphoric disorder (PDD), mood disorders due to a general medical condition, and substance-induced mood disorders; attention, learning and development disorders such as pervasive developmental disorder including autistic disorder, attention disorders including attention-deficit hyperactivity disorder (ADHD) and conduct disorder, disorders such as autism and autism spectrum disorders (including Asperger's syndrome, pervasive developmental disorder, Rett Syndrome and Fragile X Syndrome), depression, benign forgetfulness, childhood learning disorders, specific learning disorders, intellectual development disorders, and closed head injury; movement disorders and symptoms, including tremors, dyskinesia, dystonia, tics, dysphonia, ataxia, myoclonus, Essential Tremor, Tardive Dyskinesia, Restless Leg Syndrome, Tourette Syndrome, Multiple System Atrophy, Multiple Sclerosis, Huntington's Disease, Parkinson's Disease and Atypical Parkinsonisms; epilepsy; urinary incontinence; neuronal damage including ocular damage, retinopathy or macular degeneration of the eye, tinnitus, hearing impairment and loss, and brain edema; emesis; and sleep disorders including insomnia, disturbed sleep, jet lag, hypersomnia, cataplexy, sleep apnea, obstructive sleep apnea, REM sleep behavior disorder, Restless Leg Syndrome, periodic limb movement disorder, circadian rhythm sleep disorders, delayed sleep phase disorder, sleepwalking, night terrors, bed wetting, rapid eye movement sleep behavior disorder, shift work sleep disorder, excessive daytime sleepiness, non-24-hour sleep-wake disorder, sleep paralysis and narcolepsy.

In some embodiments, the neurological or psychiatric disease or disorder is Alzheimer's disease, Parkinson's disease, depression, cognitive impairment, stroke, schizophrenia, Down syndrome, or Fetal Alcohol Syndrome. In some embodiments, the neurological or psychiatric disorder is Alzheimer's disease. In some embodiments, the neurological or psychiatric disorder is Parkinson's disease. In some embodiments, the neurological or psychiatric disorder is depression. In some embodiments, the neurological or psychiatric disorder is cognitive impairment. In some embodiments, the cognitive impairment is cognitive dysfunction associated with depression, for example, major depressive disorder. In some embodiments, the neurological or psychiatric disorder is stroke. In some embodiments, the neurological or psychiatric disorder is schizophrenia. In some embodiments, the neurological or psychiatric disorder is Down syndrome. In some embodiments, the neurological or psychiatric disorder is Fetal Alcohol Syndrome.

In some embodiments, the neurological or psychiatric disease or disorder is bipolar disorder. Bipolar disorders (including both bipolar I and bipolar II) are serious psychiatric disorders that have a prevalence of approximately 2% of the population, and affects both genders alike. It is a relapsing-remitting condition characterized by cycling between elevated (i.e., manic) and depressed moods, which distinguishes it from other disorders such as major depressive disorder and schizophrenia. Bipolar I is defined by the occurrence of a full manic episode, although most individuals experience significant depression. Symptoms of mania include elevated or irritable mood, hyperactivity, grandiosity, decreased need for sleep, racing thoughts and in some cases, psychosis. The depressive episodes are characterized by anhedonia, sad mood, hopelessness, poor self-esteem, diminished concentration and lethargy. Bipolar II is defined as the occurrence of a major depressive episode and hypomanic (less severe mania) episode although subjects spend considerably more time in the depressive state. Other related conditions include cyclothymic disorder.

In some embodiments, the neurological or psychiatric disease or disorder is schizophrenia. Schizophrenia is a disorder of unknown origin, which usually appears for the first time in early adulthood and is marked by characteristics such as psychotic symptoms, phasic progression and development, and/or deterioration in social behavior and professional capability. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotoric disorders (e.g., catatonia). Other symptoms are also associated with this disorder. Schizophrenia is classified into subgroups: the paranoid type, characterized by delusions and hallucinations and absence of thought disorder, disorganized behavior, and affective flattening; the disorganized type, also named “hebephrenic schizophrenia,” in which thought disorder and flat affect are present together; the catatonic type, in which prominent psychomotor disturbances are evident, and symptoms may include catatonic stupor and waxy flexibility; and the undifferentiated type, in which psychotic symptoms are present but the criteria for paranoid, disorganized, or catatonic types have not been met. The symptoms of schizophrenia normally manifest themselves in three broad categories: positive, negative and cognitive symptoms. Positive symptoms are those which represent an “excess” of normal experiences, such as hallucinations and delusions. Negative symptoms are those where the subject suffers from a lack of normal experiences, such as anhedonia and lack of social interaction. The cognitive symptoms relate to cognitive impairment in schizophrenics, such as lack of sustained attention and deficits in decision making.

In some embodiments, the neurological or psychiatric disease or disorder is anxiety disorder. Anxiety disorders are characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs. Examples of anxiety disorders include separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, illness anxiety disorder, social (pragmatic) communication disorder, other specified anxiety disorder, and unspecified anxiety disorder; stressor-related disorders, including reactive attachment disorder, disinhibited social engagement disorder, posttraumatic stress disorder (PTSD), acute stress disorder, and adjustment disorders.

Cognitive impairment includes a decline in cognitive functions or cognitive domains, e.g., working memory, attention and vigilance, verbal learning and memory, visual learning and memory, reasoning and problem solving (e.g., executive function, speed of processing and/or social cognition). In particular, cognitive impairment may indicate deficits in attention, disorganized thinking, slow thinking, difficulty in understanding, poor concentration, impairment of problem solving, poor memory, difficulties in expressing thoughts, and/or difficulties in integrating thoughts, feelings and behavior, or difficulties in extinction of irrelevant thoughts.

In some embodiments, the neurological or psychiatric disease or disorder involves a deficit in cognition (cognitive domains as defined by the DSM-5 are: complex attention, executive function, learning and memory, language, perceptual-motor, social cognition). In some embodiments, the neurological or psychiatric disorder is associated with a deficit in dopamine signaling. In some embodiments, the neurological or psychiatric disorder is associated with basal ganglia dysfunction. In some embodiments, the neurological or psychiatric disorder is associated with dysregulated locomotor activity. In some embodiments, the neurological or psychiatric disorder is associated with impairment of prefrontal cortex functioning.

In some embodiments, the present disclosure provides a method of treating one or more symptoms of a neurological and/or psychiatric disease or disorder provided herein. Such diseases or disorders include mood disorders, including bipolar I disorder, bipolar II disorder, mania, cyclothymic disorder, substance/medication-induced bipolar and related disorders, bipolar and related disorder due to another medical condition, other specified bipolar and related disorder, and unspecified bipolar and related disorders; psychotic disorders, including schizophrenia, schizophrenia spectrum disorder, acute schizophrenia, chronic schizophrenia, NOS schizophrenia, schizoid personality disorder, schizotypal personality disorder, delusional disorder, psychosis, psychotic disorder, brief psychotic disorder, shared psychotic disorder, psychotic disorder due to a general medical condition, drug-induced psychosis (e.g., cocaine, alcohol, amphetamine), schizoaffective disorder, agitation, aggression, delirium, catalepsy, catatonia, dissociative identity disorder, paranoid personality disorder, psychotic depression, Schizotypical Personality Disorder, Childhood Disintegrative Disorder (Heller's Syndrome), Disintegrative Psychosis, Dissociative Amnesia, Somatic Symptom Disorder, Parkinson's psychosis, excitative psychosis, Tourette's syndrome, and organic or NOS psychosis; depressive disorders, including disruptive mood dysregulation disorder, major depressive disorder (MDD) (including major depressive episode), dysthymia, persistent depressive disorder (dysthymia), treatment resistant depression, premenstrual dysphoric disorder, substance/medication-induced depressive disorder, depressive disorder due to another medical condition, other specified depressive disorder, and unspecified depressive disorder; anxiety disorders; and other disorders including substance abuse or dependency (e.g., nicotine, alcohol, cocaine), addiction, internet gaming disorder, eating disorders, behavior disorder, seizure, vertigo, epilepsy, agitation, aggression, neurodegenerative disease, Alzheimer's disease, Parkinson's disease, dyskinesias, Huntington's disease, dementia, premenstrual dysphoria, attention deficit disorder (ADD) and attention deficit hyperactivity disorder (ADHD)), hyperkinetic syndrome, autism, autism spectrum disorder, obsessive-compulsive disorder, pain, fibromyalgia, migraine, cognitive impairment, movement disorder, restless leg syndrome (RLS), multiple sclerosis, Primary Progressive Multiple Sclerosis, Parkinson's disease, Huntington's disease, dyskinesias multiple sclerosis, sleep disorder, sleep apnea, narcolepsy, excessive daytime sleepiness, jet lag, drowsy side effect of medications, insomnia, sexual dysfunction, hypertension, emesis, Lesche-Nyhane disease, Wilson's disease, Rett syndrome, and Huntington's chorea. In some embodiments, the neurological and/or psychiatric disorders include agitation and aggression.

In some embodiments, the agitation and aggression are associated with Alzheimer's disease, Parkinson's disease, and/or autism.

In some embodiments, the agitation is associated with psychiatric disorders such as depression and schizophrenia.

In some embodiments, the neurological and/or psychiatric disease or disorders are obsessive-compulsive disorder and related disorders (e.g., body dysmorphic disorder, hoarding disorder, trichotillomania, excoriation disorder).

In some embodiments, the neurological and/or psychiatric diseases or disorders are disruptive, impulse-control, and conduct disorders including oppositional defiant disorder, intermittent explosive disorder, conduct disorder, antisocial personality disorder, pyromania, kleptomania, other specified disruptive, impulse-control, and conduct disorder, unspecified disruptive, impulse-control, and conduct disorder.

Depressive disorders include major depressive disorder and dysthymia, and are associated with depressed mood (sadness), poor concentration, insomnia, fatigue, appetite disturbances, excessive guilt and thoughts of suicide.

In some embodiments, the present disclosure provides a method of treating one or more symptoms including depression (e.g., major depressive disorder or dysthymia); bipolar disorder, seasonal affective disorder; cognitive deficit; sleep related disorder (e.g., sleep apnea, insomnia, narcolepsy, cataplexy) including those sleep disorders which are produced by psychiatric conditions; chronic fatigue syndrome; anxieties (e.g., general anxiety disorder, social anxiety disorder, panic disorder); obsessive compulsive disorder; post-menopausal vasomotor symptoms (e.g., hot flashes, night sweats); neurodegenerative disease (e.g., Parkinson's disease, Alzheimer's disease, amyotrophic lateral sclerosis, primary lateral sclerosis, progressive muscular atrophy, progressive bulbar (atrophy) palsy, pseudobulbar palsy spinal muscular atrophy diseases (e.g., SMA type I, also called Werdnig-Hoffmann disease, SMA type II, SMA type III, also called Kugelberg-Welander disease, and Kennedy Disease, also called progressive spinobulbar muscular atrophy), Hallervorden-Spatz disease, Seitelberger disease (Infantile Neuroaxonal Dystrophy), adrenoleukodystrophy, Alexander Disease, autosomal dominant cerebellar ataxia (ADCA), pure autonomic failure (Bradbury-Eggleston Syndrome), CADASIL Syndrome, and neuronal ceroids lipofuscinose disorders such as Batten Disease (Spielmeyer-Vogt-Sjogren)); manic disorder; dysthymic disorder; and obesity.

In some embodiments, a depressive disorder is associated with acute suicidality or suicide ideation. The United States Food and Drug Administration has adopted a “black box” label warning indicating that antidepressants may increase the risk of suicidal thinking and behavior in some children, adolescents and young adults (up to age 24) with a depressive disorder such as MDD. In some embodiments, a provided compound does not increase the risk of suicidal thinking and/or behavior in children, adolescents and/or young adults with a depressive disorder, e.g., with MDD. In some embodiments, the present disclosure provides a method of treating one or more symptoms of a depressive disorder (e.g., MDD) in children, adolescents and/or young adults without increasing the risk of suicidal thinking and/or behavior.

In some embodiments, the present disclosure provides a method of treating one or more symptoms including senile dementia, Early Onset Alzheimer's disease, Alzheimer's type dementia, cognition, memory loss, amnesia/amnestic syndrome, disturbances of consciousness, coma, lowering of attention, speech disorder, agnosia, aphasia, apraxia, Mild Cognitive Impairment (MCI), benign forgetfulness, mild neurocognitive disorder, major neurocognitive disorder, neurocognitive disorder due to disease (e.g., Huntington's Disease, Parkinson's disease, Prion Disease, Traumatic Brain Injury, HIV or AIDS), Binswanger's Disease (subcortical leukoencephalopathy), and Capgras Syndrome.

In some embodiments, the present disclosure provides a method of treating one or more symptoms of pain, e.g., neuropathic pain, sensitization accompanying neuropathic pain, or inflammatory pain. In some embodiments, the pain is neuropathic pain, including post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use). In some embodiments, the pain is acute pain, nociceptive pain, arthritis pain, rheumatoid arthritis, osteoarthritis, joint pain, muscoskeletal pain, back pain, dorsalgia, bulging disc, hip pain, visceral pain, headache, tension headache, acute tension headache, chronic tension headache, chronic cluster headache, common migraine, classic migraine, cluster headache, mixed headache, posttraumatic headache, eye strain headache, Short-lasting Unilateral Neuralgiform (SUNCT) headache, SUNCT Syndrome, herpes zoster, acute herpes zoster, shingles, postherpetic neuralgia (shingles), causalgia, central pain, central pain syndrome, chronic back pain, neuralgia, neuropathic pain syndrome, neuropathy, diabetic neuropathy, diabetes-related neuropathy, diabetes-related nerve pain, fibrositis, peripheral neuropathy caused by chemotherapy, peripheral nerve disease, peripheral neuropathy, nerve pain, nerve trauma, sensitization accompanying neuropathic pain, complex regional pain syndrome, compression neuropathy, craniofacial pain, chronic joint pain, chronic knee pain, chronic pain syndrome, cancer pain, trigeminal neuralgia, tic doloreaux, reflex sympathetic causalgia, painful peripheral neuropathy, spinal nerve injury, arachnoiditis, spinal pain, Bernhardt-Roth Syndrome (meralgia parasthetica), carpal tunnel syndrome, cerebrospinal fluid syndrome, Charcot-Marie-tooth disease, hereditary motor and sensory neuropathy, peroneal muscular atrophy, cluster-tic syndrome, coccygeal pain syndromes, compartment syndrome, degenerative disc disease, failed back surgery syndrome, genito-pelvic pain/penetration disorder, gout, inflammatory pain, lumbar radiculopathy, neuroma (painful scar), pain associated with multiple sclerosis, pelvic floor disorders, phantom limb pain, piriformis syndrome, psychogenic pain, radicular pain syndrome, Raeder's syndrome, referred pain, reflex sympathetic dystrophy syndrome, sciatica, sciatica pain, scoliosis, slipped disc, somatic pain, spinal stenosis, stiff-person syndrome/stiff-man syndrome, stump pain, sympathetically maintained pain, tolosa-hunt syndrome, whiplash, or pain associated with Lyme disease.

In some embodiments, the present disclosure provides a method of treating one or more symptoms including obesity; migraine or migraine headache; and sexual dysfunction, in men or women, including without limitation sexual dysfunction caused by psychological and/or physiological factors, erectile dysfunction, premature ejaculation, vaginal dryness, lack of sexual excitement, inability to obtain orgasm, and psycho-sexual dysfunction, including without limitation, inhibited sexual desire, inhibited sexual excitement, inhibited female orgasm, inhibited male orgasm, functional dyspareunia, functional vaginismus, and atypical psychosexual dysfunction.

In some embodiments, the present disclosure provides a method of suppressing rapid eye movement (REM) during both sleep and daytime equivalent.

In some embodiments, the present disclosure provides a method of suppressing or eliminating pathological or excessive REM during the night or daytime equivalent.

In some embodiments, the present disclosure provides a method of treating one or more symptoms including cataplexy (sudden involuntary transient bouts of muscle weakness or paralysis while awake); nighttime sleep disturbance/sleep fragmentation associated with narcolepsy or other conditions; sleep paralysis associated with narcolepsy or other conditions; hypnagogic and hypnapompic hallucinations associated with narcolepsy or other conditions; and excessive daytime sleepiness associated with narcolepsy, sleep apnea or shift work disorder and other medical conditions such as cancer, chronic fatigue syndrome and fibromyalgia.

In some embodiments, the present disclosure provides a method of treating one or more symptoms of movement diseases or disorders, including akinesias, akinetic-rigid syndromes, dyskinesias and dystonias. Examples of akinesias and akinetic-rigid syndromes include Parkinson's disease, drug-induced Parkinsonism, postencephalitic Parkinsonism, secondary Parkinsonism, Parkinson plus syndromes, atypical Parkinsonism, idiopathic Parkinsonism, progressive supranuclear palsy, multiple system atrophy, corticobasal degeneration, Parkinsonism-ALS dementia complex and basal ganglia calcification, medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors. Examples of dyskinesias include drug (e.g. L-DOPA) induced dyskinesia tremor (such as rest tremor, postural tremor, intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics). Examples of dystonias include generalized dystonia, idiopathic dystonia, drug-induced dystonia, symptomatic dystonia, paroxymal dystonia, focal dystonia, blepharospasm, oromandibular dystonia, spasmodic dysphonia, spasmodic torticollis, axial dystonia, dystonic writer's cramp and hemiplegic dystonia. Other examples of movement diseases or disorders include stereotypic movement disorder, persistent (chronic) motor disorder, medication-Induced movement disorder, psychogenic movement disorders, substance/medication-induced movement disorder, extrapyramidal movement disorders, hyperkinetic movement disorders, hypokinetic movement disorders, alternating hemiplegia, Angelman syndrome, Hallervorden-Spatz Disease, ataxia, dentate cerebellar ataxia, ataxia telangiectasia (Louis-Bar syndrome), Friedreich's Ataxia, hereditary spinal ataxia, hereditary spinal sclerosis, Machado-Joseph Disease, spinocerebellar ataxia, progressive myoclonic ataxia, athetosis, ballismus, blepharospasm (eye twitching), cerebral palsy, tardive dystonia, tardive dyskinesia, idiopathic torsion dystonia, torsion dystonia, focal dystonia, idiopathic familial dystonia, Idiopathic nonfamilial dystonia, cervical dystonia (spasmodic torticollis), primary dystonia, orofacial dystonia, developmental coordination disorder, bulbospinal muscular atrophy (Kennedy's Disease), Shy-Drager Syndrome, and Stiff-Person (Stiff-Man) Syndrome.

In some embodiments, the present disclosure provides a method of treating one or more symptoms of epilepsy and/or seizures, including abdominal epilepsy, absence seizure, acquired epilepsy, acquired epileptiform aphasia, Aicardi syndrome, Alpers' disease, Alpers-Huttenlocher syndrome, Angelman syndrome, benign focal epilepsy, benign focal epilepsy of childhood, benign intracranial hypertension, benign rolandic epilepsy (BRE), CDKL5 disorder, childhood absence epilepsy, dentate cerebellar ataxia, Doose syndrome, Dravet syndrome, dyscognitive focal seizure, epilepsy with grand mal seizures, epilepsy with myoclonic-absences, epileptic hemiplegia, febrile seizures, focal seizure, frontal lobe epilepsy, generalized tonic-clonic seizures, genetic epilepsy, Glutl deficiency syndrome, hypothalamic hamartoma, idiopathic epilepsy, idiopathic generalized epilepsy, idiopathic localization-related epilepsies, idiopathic partial epilepsy, idiopathic seizure, juvenile absence epilepsy, juvenile myoclonic epilepsy, Lafora disease, Lafora progressive myoclonus epilepsy, Landau-Kleffner syndrome, Lassueur-Graham-Little syndrome, Lennox syndrome, Lennox-Gastaut syndrome, medically refractory epilepsy, mesial-temporal lobe sclerosis, myoclonic seizure, neonatal epilepsy, occipital lobe epilepsy, Ohtahara syndrome, Panayiotopoulos syndrome, parietal lobe epilepsy, PCDH19 epilepsy, photosensitive epilepsy, progressive myoclonic epilepsies, Rasmussen's encephalitis, Rasmussen's syndrome, refractory epilepsy, seizure disorder, status epilepticus, Sturge-Weber syndrome, symptomatic generalized epilepsy, symptomatic partial epilepsy, TBCK-related ID syndrome, temporal lobe epilepsy, temporal lobe seizures, tonic-clonic seizure, West syndrome, tremor, cerebellar tremor, cerebellar outflow tremor, intention tremor, essential tremor, benign essential tremor, Parkinsonian tremor, and medication-induced postural tremor.

### Pharmaceutical Compositions

According to an embodiment, the disclosure provides a composition comprising Compound 1 or salts thereof (or crystalline forms, hydrates, or solvates thereof) and a pharmaceutically acceptable carrier, adjuvant, or vehicle. In some embodiments, the amount of Compound 1 in compositions of this disclosure is such that is effective to treat, prevent, and/or manage various neurological and/or psychiatric diseases, disorders and/or symptoms in a subject. In some embodiments, a composition of this disclosure is formulated for administration to a subject in need of such composition. In some embodiments, a composition of this disclosure is formulated for oral administration to a subject.

As used herein, the term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

In certain embodiments, provided herein is a composition (e.g., a pharmaceutical composition) comprising Compound 1 or salts thereof (or crystalline forms, hydrates, or solvates thereof) and a pharmaceutically acceptable excipient or carrier. In some embodiments, provided herein is a method of treating neurological or psychiatric diseases and disorders in a subject in need thereof in a subject, comprising administering an effective amount of Compound 1 or salts thereof (or crystalline forms, hydrates, or solvates thereof) or a pharmaceutical composition described herein. Examples of carriers and excipients are well known to those skilled in the art and are described in detail in, e.g., Ansel, Howard C, et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present disclosure or pharmaceutical composition thereof) or aid in the manufacturing of the pharmaceutical product (i.e., medicament).

Compositions of the present disclosure may be administered orally, parenterally, by inhalation, topically, rectally, nasally, buccally, sublingually, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. Pharmaceutically acceptable compositions of this disclosure may be orally administered in any orally acceptable dosage form including capsules, tablets, aqueous suspensions or solutions.

The amount of Compound 1 or salts thereof (or crystalline forms, hydrates, or solvates thereof) that may be combined with the carrier materials to produce a composition in a single dosage form will vary depending upon a variety of factors, including the host treated and the particular mode of administration. It should also be understood that a specific dosage and treatment regimen for any particular subject will depend upon a variety of factors, including the activity of the specific form of Compound 1 employed, the age, body weight, general health, sex, diet, time of administration, rate of excretion, drug combination, and the judgment of the treating physician and the severity of the particular disease being treated.

### Combination Therapies

In some embodiments, the present disclosure provides a method of treating a neurological and/or psychiatric disease or disorder described herein, comprising administering a compound of the disclosure in conjunction with one or more pharmaceutical agents. Suitable pharmaceutical agents that may be used in combination with Compound 1 or salts thereof (or crystalline forms, hydrates, or solvates thereof) include anti-Parkinson's drugs, anti-Alzheimer's drugs, antidepressants, anti-psychotics, anti-ischemics, CNS depressants, anti-cholinergics, nootropics, epilepsy medication, attention (e.g., ADD/ADHD) medications, sleep-promoting medications, wakefulness-promoting medications, and pain medications. In some embodiments, suitable pharmaceutical agents are anxiolytics.

Suitable anti-Parkinson's drugs include dopamine replacement therapy (e.g. L-DOPA, carbidopa, COMT inhibitors such as entacapone or tolcapone), dopamine agonists (e.g. D1 agonists, D2 agonists, mixed D1/D2 agonists, bromocriptine, pergolide, cabergoline, ropinirole, pramipexole, piribedil, or apomorphine in combination with domperidone), histamine H2 antagonists, monoamine oxidase inhibitors (such as selegiline, rasagiline, safinamide and tranylcypromine), certain atypical antipsychotics such as pimavanserin (a non-dopaminergic atypical antipsychotic and inverse agonist of the serotonin 5-HT2A receptor), and amantadine.

In some embodiments, Compound 1 or salts thereof (or crystalline forms, hydrates, or solvates thereof) can be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone or tolcapone, MAO A/B inhibitors, antioxidants, A2a adenosine receptor antagonists, cholinergic agonists, MDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer's drugs include beta-secretase inhibitors, gamma-secretase inhibitors, cholinesterase inhibitors such as donepezil, galantamine or rivastigmine, HMG-CoA reductase inhibitors, NSAID's including ibuprofen, vitamin E, and anti-amyloid antibodies. In some embodiments, an anti-Alzheimer's drug is memantine.

Suitable anti-depressants and anti-anxiety agents include norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, a-adrenoreceptor antagonists, neurokinin-1 receptor antagonists, atypical anti-depressants, benzodiazepines, 5-HT1 A agonists or antagonists, especially 5-HT1 A partial agonists, and corticotropin releasing factor (CRF) antagonists.

Specific suitable anti-depressant and anti-anxiety agents include amitriptyline, clomipramine, doxepin, imipramine and trimipramine; amoxapine, desipramine, citalopram, escitalopram, maprotiline, nortriptyline and protriptyline; fluoxetine, fluvoxamine, paroxetine and sertraline; isocarboxazid, phenelzine, tranylcypromine and selegiline; moclobemide: venlafaxine; desvenlafaxine, duloxetine; aprepitant; bupropion, vilazodone, mirtazapine, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, reboxetine, vortioxetine, clorazepate, and ketamine and pharmaceutically acceptable salts thereof. In some embodiments, suitable anti-depressant and anti-anxiety agents are tianeptine, or pharmaceutically acceptable salts thereof.

Suitable anti-psychotic and mood stabilizer agents include D2 antagonists, 5HT2A antagonists, atypical antipsychotics, lithium, and anticonvulsants.

Specific suitable anti-psychotic and mood stabilizer agents include chlorpromazine, fluphenazine, haloperidol, amisulpride, perphenazine, thioridazine, trifluoperazine, aripiprazole, asenapine, clozapine, olanzapine, paliperidone, brexpiprazole, paliperidone, cariprazine, pimavanserin, illoperidone, lumateperone, MIN-101, quetiapine, risperidone, ziprasidone, lurasidone, flupentixol, levomepromazine, pericyazine, perphenazine, pimozide, prochlorperazine, zuclopenthixol, olanzapine and fluoxetine, lithium, carbamazepine, lamotrigine, valproic acid, iloperidone, thiothixene, gabapentin, tiagabine and pharmaceutically acceptable salts thereof.

Suitable epilepsy medications include levetiracetam, oxcarbazepine, clobazam, retigabine, zonisamide, felbamate, esclicarbazepine acetate, lacosamide, carbamazepine, tiagabine, methsuximide, progabide, valproic acid, lamotrigine, brivaracetam, rufinamide, topiramate and perampanel.

Suitable attention medications include methyl phenidate, atomoxetine, guanfacine, D-amphetamine, lisdexamphetamine, methylamphetamine, and clonidine.

Suitable sleep-promoting medications include ramelteon, triazolam, zopiclone, eszopiclone, Zolpidem, temazepam, and trazodone.

Suitable wakefulness-promoting medications include Modafinil, D-Amphetamine, caffeine, and armodafinil.

Suitable pain medications include dextromethorphan, tapentadol, buprenorphine, codeine, fentanyl, hydrocodone, hydromorphone, morphine, naloxegol, oxycodone, tramadol, gabapentil, difluprednate, pregabalin, acetyl salicyclic acid, bromfenac, diclofenac, diflunisal, indomethacin, ketorolac, meoxican, and naproxen.

In some embodiments, compounds and compositions of the disclosure may be used in combination with other therapies. Suitable therapies include psychotherapy, cognitive behavioral therapy, electroconvulsive therapy, transcranial magnetic stimulation, vagus nerve stimulation, and deep-brain stimulation.

The exact amount required will vary from subject to subject, depending on the species, age, and general condition of the subject, the severity of the condition, the particular agent, its mode of administration, and the like. The compounds and compositions of the disclosure are preferably formulated in dosage unit form for ease of administration and uniformity of dosage. The expression “dosage unit form” as used herein refers to a physically discrete unit of agent appropriate for the subject to be treated. It will be understood, however, that the total daily usage of the compounds and compositions of the present disclosure will be decided by the attending physician within the scope of sound medical judgment.

The pharmaceutically acceptable compositions of this disclosure can be administered to humans and other animals orally, rectally, parenterally, intracisternally, intravaginally, intraperitoneally, topically (as by powders, ointments, or drops), bucally, sublingually, as an oral or nasal spray, or the like, depending on the severity of the infection being treated. In some embodiments, Compound 1 or salts thereof (or crystalline forms, hydrates, or solvates thereof) may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

In some embodiments, a combination of two or more therapeutic agents may be administered together with Compound 1 or salts thereof (or crystalline forms, hydrates, or solvates thereof). In some embodiments, a combination of three or more therapeutic agents may be administered with Compound 1 or salts thereof (or crystalline forms, hydrates, or solvates thereof).

Other examples of agents the compounds and compositions of this disclosure may also be combined with include: vitamins and nutritional supplements, antiemetics (e.g. 5-HT3 receptor antagonists, dopamine antagonists, K1 receptor antagonists, histamine receptor antagonists, cannabinoids, benzodiazepines, or anticholinergics), agents for treating Multiple Sclerosis (MS) such as beta interferon (e.g., Avonex® and Rebif®, dalfampridine, alemtuzumab), Copaxone®, and mitoxantrone; treatments for Huntington's disease such as tetrabenazine; treatments for asthma such as albuterol and Singulair®; anti-inflammatory agents such as corticosteroids, T F blockers, IL-1 RA, azathioprine, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspoglutide), gastric inhibitory peptide analogs, DPP-4 inhibitors (vildagliptin, sitagliptin, saxagliptin, linagliptin, alogliptin), amylin analogs (pramlintide), and insulin and insulin analogs.

In some embodiments, Compound 1 or salts thereof (or crystalline forms, hydrates, or solvates thereof) are administered in combination with an antisense agent, a monoclonal or polyclonal antibody, or an siRNA therapeutic.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another, normally within five hours from one another.

As used herein, the term “combination,” “combined,” and related terms refers to the simultaneous or sequential administration of therapeutic agents in accordance with this disclosure. For example, Compound 1 or salts thereof (or crystalline forms, hydrates, or solvates thereof) may be administered with another therapeutic agent simultaneously or sequentially in separate unit dosage forms or together in a single unit dosage form. Accordingly, the present disclosure provides a single unit dosage form comprising Compound 1 or salts thereof (or crystalline forms, hydrates, or solvates thereof), an additional therapeutic agent, and a pharmaceutically acceptable carrier, adjuvant, or vehicle.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.01-100 mg/kg body weight/day of an inventive can be administered.

In those compositions which comprise an additional therapeutic agent, that additional therapeutic agent and Compound 1 or salts thereof (or crystalline forms, hydrates, or solvates thereof) may act synergistically. Therefore, the amount of additional therapeutic agent in such compositions will be less than that required in a monotherapy utilizing only that therapeutic agent. In such compositions a dosage of between 0.01-100 mg/kg body weight/day of the additional therapeutic agent can be administered.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the present disclosure provides a medicament comprising Compound 1 or salts thereof (or crystalline forms, hydrates, or solvates thereof), and a pharmaceutically acceptable carrier, adjuvant, or vehicle.

In some embodiments, the present disclosure provides the use of Compound 1 or salts thereof (or crystalline forms, hydrates, or solvates thereof) in the manufacture of a medicament for the treatment of a neurological and/or psychiatric disease or disorder.

### EXAMPLES

### Example 1: Preparation of Salts of Compound 1

The free base of Compound 1 (500 mg) was dissolved in MeOH (34 mL). The solution was divided into 33 vials (resulting in a 15 mg scale of Compound 1). To each vial, the appropriate counter ion (0.95 equivalents) was added and dissolved by the screening solvent listed in Table 1. The solution was heated at 60° C. for 1 h and then allowed to cool undisturbed at room temperature. Each vial was opened and allowed to stand at room temperature (slow evaporation). In several cases, acetone or EtOAc additive were added to vials and allowed to stand at room temperature, allowing for slow evaporation. Results of the solvent screen are shown below in Table 1. Further, certain experiments were performed on a larger scale, as shown in Table 1.

TABLE 1
Solvent screen

| | Acid (0.95 | | | Solvent | |
|---|---|---|---|---|---|
| | equivalents | Scale | | Additive | |
| | rel. to | (amount | | for slow | |
| | Compound | of Com- | Screening | evapora- | Notes On |
| Entry | 1) | pound 1) | Solvent | tion | Product |
| 1 | HCl | 15 mg | MeOH | — | — |
| 2 | HCl | 15 mg | THF | — | Crystalline - |
| | | | | | Form HA |
| 3 | HCl | 15 mg | EtOAc | — | Crystalline - |
| | | | | | Form HA |
| 4 | H₃PO₄ | 15 mg | MeOH + | — | Crystalline |
| | | | Acetone | | |
| 5 | H₃PO₄ | 15 mg | THF | — | Crystalline |
| 6 | H₃PO₄ | 15 mg | EtOAc | — | Crystalline |
| 7 | H₃PO₄ | 200 mg | MeOH (1 mL) + | ace- | Crystalline |
| | | | EtOAc (5 mL) | tone | |
| 8 | H₃PO₄ | 1000 mg | MeOH (7 mL) + | ace- | Crystalline |
| | | | EtOAc (35 mL) | tone | |
| 9 | L-Tartaric | 15 mg | MeOH + | | Crystalline - |
| | acid | | acetone | | Form LB |
| 10 | L-Tartaric | 15 mg | THF | | Crystalline - |
| | acid | | | | Form LA |
| 11 | L-Tartaric | 15 mg | EtOAc | | Crystalline - |
| | acid | | | | Form LA |
| 12 | L-Tartaric | 100 mg | MeOH (1 mL) + | ace- | Crystalline - |
| | acid | | EtOAc (5 mL), | tone | Form LC |
| | | | 100 mg scale | | |
| 13 | L-Tartaric | 100 mg | MeOH (1 mL) + | EtOAc | Crystalline - |
| | acid | | EtOAc (5 mL); | | Form LC |
| | | | 100 mg scale | | |
| 14 | D-Tartaric | 15 mg | MeOH + | | — |
| | acid | | acetone | | |
| 15 | D-Tartaric | 15 mg | THF | | Crystalline |
| | acid | | | | |
| 16 | D-Tartaric | 15 mg | EtOAc | | Crystalline |
| | acid | | | | |
| 17 | Fumaric | 15 mg | MeOH | | Crystalline - |
| | acid | | | | Form FA |
| 18 | Fumaric | 15 mg | THF | | Crystalline - |
| | acid | | | | Form FA |
| 19 | Fumaric | 15 mg | EtOAc | | Crystalline - |
| | acid | | | | Form FB |
| 20 | Fumaric | 200 mg | MeOH (1 mL) + | Ace- | Crystalline - |
| | acid | | Acetone (5 mL) | tone | Form FA |
| 21 | Citric Acid | 15 mg | MeOH + | | Crystalline |
| | | | acetone | | |
| 22 | Citric Acid | 15 mg | THF | | — |
| 23 | Citric Acid | 15 mg | EtOAc | | Crystalline |
| 24 | Citric Acid | 100 mg | MeOH (1 mL) + | Ace- | Crystalline |
| | | | EtOAc (5 mL); | tone | |
| 25 | TsOH | 15 mg | MeOH + | | — |
| | | | acetone | | |
| 26 | TsOH | 15 mg | THF | | — |
| 27 | TsOH | 15 mg | EtOAc | | — |
| 28 | Succinic | 15 mg | MeOH + | | — |
| | acid | | acetone | | |
| 29 | Succinic | 15 mg | THF | | Crystalline |
| | acid | | | | |
| 30 | Succinic | 15 mg | EtOAc | | Crystalline |
| | acid | | | | |
| 31 | Glutaric | 15 mg | MeOH + | | Crystalline |
| | acid | | acetone | | |
| 32 | Glutaric | 15 mg | THF | | — |
| | acid | | | | |
| 33 | Glutaric | 15 mg | EtOAc | | — |
| | acid | | | | |
| 34 | L-malic | 15 mg | MeOH | | Crystalline |
| | acid | | | | |
| 35 | L-malic | 15 mg | THF | | Crystalline |
| | acid | | | | |
| 36 | L-malic | 15 mg | EtOAC | | Crystalline |
| | acid | | | | |
| 37 | BsOH | 15 mg | MeOH + | | — |
| | | | acetone | | |
| 38 | BsOH | 15 mg | THF | | — |
| 39 | BsOH | 15 mg | EtOAc | | Crystalline |

### Example 2. Synthesis of Free Base

Saturated sodium bicarbonate water (15 ml) and water (5 ml) was added to Compound 1 hydrochloride (600 mg). The solution was extracted with chloroform (15 mL, 3 times). The organic extract was washed with brine (20 ml) and evaporated. The free base of Compound 1 was isolated as an oil (approx. 500 mg).

### Example 3. Compound 1 Hydrochloride Form HA

The XRPD spectrum for Compound 1 Hydrochloride HA is provided in FIG. 1 (FIG. 1) and the corresponding peak data is provided below in Table 2.

TABLE 2
XRPD Peak Data for Compound 1 Hydrochloride Form HA

| | FWHM | | d- | | Rel. |
|---|---|---|---|---|---|
| Pos. | Left | Area | spacing | Height | Int. |
| [°2θ] | [°2θ] | [cts * °2θ] | [Å] | [cts] | [%] |
| 6.7 | 0.1 | 7.0 | 13.1 | 43.1 | 0.6 |
| 8.6 | 0.2 | 46.6 | 10.3 | 214.3 | 2.7 |
| 9.4 | 0.1 | 1072.7 | 9.4 | 7887.4 | 100.0 |
| 10.3 | 0.2 | 24.8 | 8.6 | 91.1 | 1.2 |
| 11.4 | 0.1 | 196.4 | 7.7 | 1443.9 | 18.3 |
| 12.5 | 0.3 | 16.7 | 7.1 | 38.4 | 0.5 |
| 13.6 | 0.2 | 11.6 | 6.5 | 53.1 | 0.7 |
| 14.2 | 0.1 | 192.1 | 6.2 | 1412.5 | 17.9 |
| 15.1 | 0.1 | 328.7 | 5.9 | 2417.1 | 30.6 |
| 16.0 | 0.2 | 9.9 | 5.5 | 45.7 | 0.6 |
| 17.2 | 0.1 | 329.2 | 5.2 | 2420.3 | 30.7 |
| 17.6 | 0.1 | 442.4 | 5.0 | 2710.9 | 34.4 |
| 17.9 | 0.2 | 16.6 | 5.0 | 76.4 | 1.0 |
| 18.8 | 0.1 | 87.4 | 4.7 | 535.5 | 6.8 |
| 19.2 | 0.1 | 116.1 | 4.6 | 609.6 | 7.7 |
| 20.1 | 0.2 | 57.1 | 4.4 | 262.4 | 3.3 |
| 20.6 | 0.1 | 30.9 | 4.3 | 189.5 | 2.4 |
| 20.9 | 0.2 | 37.8 | 4.3 | 173.5 | 2.2 |
| 21.5 | 0.2 | 25.4 | 4.1 | 93.5 | 1.2 |
| 22.1 | 0.1 | 49.6 | 4.0 | 303.9 | 3.9 |
| 23.1 | 0.1 | 55.2 | 3.8 | 405.7 | 5.1 |
| 23.6 | 0.1 | 21.2 | 3.8 | 129.9 | 1.7 |
| 24.3 | 0.1 | 204.0 | 3.7 | 1250.3 | 15.9 |
| 24.8 | 0.1 | 41.2 | 3.6 | 303.0 | 3.8 |
| 25.2 | 0.1 | 24.4 | 3.5 | 149.6 | 1.9 |
| 25.6 | 0.1 | 76.5 | 3.5 | 562.3 | 7.1 |
| 25.8 | 0.1 | 170.3 | 3.4 | 894.7 | 11.3 |
| 26.2 | 0.2 | 269.3 | 3.4 | 824.9 | 10.5 |
| 27.0 | 0.1 | 615.1 | 3.3 | 3768.7 | 47.8 |
| 27.7 | 0.1 | 66.1 | 3.2 | 405.1 | 5.1 |
| 28.0 | 0.1 | 38.5 | 3.2 | 283.1 | 3.6 |
| 28.3 | 0.1 | 90.9 | 3.2 | 668.5 | 8.5 |
| 28.6 | 0.1 | 99.9 | 3.1 | 734.3 | 9.3 |
| 29.8 | 0.1 | 27.2 | 3.0 | 166.4 | 2.1 |
| 30.1 | 0.1 | 31.8 | 3.0 | 195.0 | 2.5 |
| 30.5 | 0.1 | 79.8 | 2.9 | 488.7 | 6.2 |
| 31.7 | 0.1 | 83.3 | 2.8 | 612.1 | 7.8 |
| 32.2 | 0.2 | 58.9 | 2.8 | 270.7 | 3.4 |
| 32.8 | 0.2 | 40.3 | 2.7 | 148.3 | 1.9 |
| 33.0 | 0.1 | 8.7 | 2.7 | 53.1 | 0.7 |
| 33.5 | 0.2 | 22.6 | 2.7 | 69.3 | 0.9 |
| 34.0 | 0.2 | 20.0 | 2.6 | 73.6 | 0.9 |
| 34.5 | 0.1 | 17.1 | 2.6 | 104.6 | 1.3 |
| 35.6 | 0.1 | 174.9 | 2.5 | 1286.0 | 16.3 |
| 36.4 | 0.1 | 16.7 | 2.5 | 102.1 | 1.3 |
| 38.3 | 0.2 | 42.6 | 2.3 | 195.5 | 2.5 |
| 38.6 | 0.2 | 41.2 | 2.3 | 189.3 | 2.4 |
| 39.5 | 0.1 | 16.5 | 2.3 | 100.9 | 1.3 |

A DSC thermogram for Form HA is shown in FIG. 2 (FIG. 2). The thermogram is characterized by endotherm peaks at temperatures of about 99° C. and about 187° C. FIG. 3 (FIG. 3) shows a thermogravimetric analysis (TGA) thermogram of Form HA. FIG. 4 (FIG. 4) shows a dynamic vapor sorption (DVS) isotherm of Form HA.

### Example 4. Compound 1 Hydrochloride Form HB

The XRPD spectrum for Compound 1 Hydrochloride HB is provided in FIG. 5 (FIG. 5) and the corresponding peak data is provided below in Table 3.

TABLE 3
XRPD Peak Data for Compound 1 Hydrochloride Form HB

| | FWHM | | d- | | Rel. |
|---|---|---|---|---|---|
| Pos. | Left | Area | spacing | Height | Int. |
| [°2θ] | [°2θ] | [cts * °2θ] | [Å] | [cts] | [%] |
| 8.6 | 0.2 | 139.5 | 10.2 | 640.9 | 28.7 |
| 9.6 | 0.1 | 189.2 | 9.2 | 1391.2 | 62.3 |
| 10.3 | 0.1 | 364.2 | 8.6 | 2231.9 | 100.0 |
| 12.6 | 0.1 | 146.9 | 7.0 | 1079.8 | 48.4 |
| 13.6 | 0.2 | 57.1 | 6.5 | 174.8 | 7.8 |
| 14.7 | 0.2 | 314.0 | 6.0 | 1442.9 | 64.7 |
| 16.5 | 0.1 | 99.9 | 5.4 | 524.9 | 23.5 |
| 17.3 | 0.1 | 296.0 | 5.1 | 2176.4 | 97.5 |
| 17.6 | 0.2 | 80.4 | 5.0 | 369.3 | 16.6 |
| 18.3 | 0.1 | 67.1 | 4.8 | 493.1 | 22.1 |
| 19.5 | 0.1 | 45.3 | 4.6 | 277.8 | 12.5 |
| 19.7 | 0.1 | 44.1 | 4.5 | 269.9 | 12.1 |
| 20.1 | 0.2 | 75.8 | 4.4 | 278.7 | 12.5 |
| 21.1 | 0.2 | 45.3 | 4.2 | 208.2 | 9.3 |
| 22.0 | 0.3 | 82.6 | 4.0 | 216.9 | 9.7 |
| 23.1 | 0.2 | 55.5 | 3.9 | 255.0 | 11.4 |
| 23.8 | 0.1 | 188.1 | 3.7 | 988.1 | 44.3 |
| 24.4 | 0.2 | 239.1 | 3.6 | 732.6 | 32.8 |
| 25.3 | 0.1 | 98.4 | 3.5 | 516.6 | 23.1 |
| 26.3 | 0.2 | 116.7 | 3.4 | 536.5 | 24.0 |
| 26.6 | 0.1 | 60.5 | 3.4 | 370.5 | 16.6 |
| 26.9 | 0.1 | 151.8 | 3.3 | 1116.3 | 50.0 |
| 27.1 | 0.1 | 154.7 | 3.3 | 1137.6 | 51.0 |
| 27.4 | 0.1 | 102.1 | 3.3 | 625.5 | 28.0 |
| 28.9 | 0.2 | 112.0 | 3.1 | 514.8 | 23.1 |
| 29.7 | 0.2 | 38.9 | 3.0 | 119.2 | 5.3 |
| 30.3 | 0.2 | 52.0 | 2.9 | 238.7 | 10.7 |
| 31.7 | 0.2 | 75.6 | 2.8 | 278.1 | 12.5 |
| 32.6 | 0.2 | 45.0 | 2.7 | 165.5 | 7.4 |
| 33.3 | 0.5 | 87.3 | 2.7 | 133.7 | 6.0 |
| 34.8 | 0.3 | 85.5 | 2.6 | 196.5 | 8.8 |
| 35.6 | 0.2 | 59.0 | 2.5 | 271.3 | 12.2 |
| 38.8 | 0.2 | 42.7 | 2.3 | 130.8 | 5.9 |

FIG. 6 (FIG. 6) shows a dynamic vapor sorption (DVS) isotherm of Form HB.

### Example 5. Compound 1 Phosphate

The XRPD spectrum for Compound 1 Phosphate is provided in FIG. 7 (FIG. 7) and the corresponding peak data is provided below in Table 4.

TABLE 4
XRPD Peak Data for Compound 1 Phosphate

| | | | d- | | Rel. |
|---|---|---|---|---|---|
| | FWHM | Area | spacing | Height | Int. |
| Pos. [°2θ] | Left [°2θ] | [cts * °2θ] | [Å] | [cts] | [%] |
| 4.6 | 0.1 | 1876.4 | 19.4 | 13796.8 | 99.4 |
| 9.1 | 0.1 | 665.0 | 9.7 | 4890.0 | 35.2 |
| 13.6 | 0.1 | 36.5 | 6.5 | 335.3 | 2.4 |
| 14.0 | 0.1 | 40.3 | 6.3 | 296.0 | 2.1 |
| 15.2 | 0.1 | 10.6 | 5.8 | 97.6 | 0.7 |
| 15.7 | 0.1 | 79.4 | 5.6 | 583.5 | 4.2 |
| 16.2 | 0.1 | 28.8 | 5.5 | 264.9 | 1.9 |
| 18.2 | 0.1 | 1887.7 | 4.9 | 13880.4 | 100.0 |
| 19.1 | 0.1 | 59.7 | 4.6 | 548.4 | 4.0 |
| 19.8 | 0.1 | 9.5 | 4.5 | 87.6 | 0.6 |
| 21.2 | 0.1 | 20.5 | 4.2 | 187.9 | 1.4 |
| 22.3 | 0.1 | 77.0 | 4.0 | 707.8 | 5.1 |
| 22.8 | 0.1 | 1005.6 | 3.9 | 9242.6 | 66.6 |
| 23.1 | 0.1 | 44.5 | 3.8 | 408.6 | 2.9 |
| 23.4 | 0.1 | 24.7 | 3.8 | 181.9 | 1.3 |
| 24.0 | 0.1 | 12.4 | 3.7 | 75.8 | 0.6 |
| 24.8 | 0.1 | 82.3 | 3.6 | 605.3 | 4.4 |
| 25.2 | 0.1 | 33.0 | 3.5 | 242.5 | 1.8 |
| 25.5 | 0.1 | 16.6 | 3.5 | 152.6 | 1.1 |
| 26.0 | 0.1 | 121.0 | 3.4 | 1111.9 | 8.0 |
| 26.8 | 0.1 | 12.3 | 3.3 | 150.8 | 1.1 |
| 27.0 | 0.1 | 17.7 | 3.3 | 130.0 | 0.9 |
| 27.4 | 0.1 | 154.5 | 3.3 | 1419.7 | 10.2 |
| 27.7 | 0.1 | 20.0 | 3.2 | 183.7 | 1.3 |
| 28.2 | 0.1 | 14.7 | 3.2 | 134.9 | 1.0 |
| 29.4 | 0.1 | 10.5 | 3.0 | 64.5 | 0.5 |
| 29.6 | 0.1 | 9.1 | 3.0 | 55.9 | 0.4 |
| 30.1 | 0.1 | 103.4 | 3.0 | 760.0 | 5.5 |
| 30.7 | 0.1 | 14.0 | 2.9 | 128.2 | 0.9 |
| 31.0 | 0.1 | 59.6 | 2.9 | 438.6 | 3.2 |
| 31.3 | 0.1 | 12.9 | 2.9 | 79.2 | 0.6 |
| 32.1 | 0.1 | 41.8 | 2.8 | 384.3 | 2.8 |
| 33.5 | 0.2 | 9.1 | 2.7 | 28.0 | 0.2 |
| 34.1 | 0.1 | 24.0 | 2.6 | 176.4 | 1.3 |
| 34.9 | 0.1 | 32.7 | 2.6 | 300.7 | 2.2 |
| 35.2 | 0.1 | 16.5 | 2.5 | 151.3 | 1.1 |
| 36.4 | 0.1 | 8.2 | 2.5 | 50.3 | 0.4 |
| 36.8 | 0.1 | 25.9 | 2.4 | 190.1 | 1.4 |
| 37.8 | 0.1 | 18.5 | 2.4 | 135.8 | 1.0 |
| 38.3 | 0.1 | 16.1 | 2.3 | 118.0 | 0.9 |
| 38.8 | 0.1 | 62.1 | 2.3 | 456.3 | 3.3 |
| 39.0 | 0.1 | 75.9 | 2.3 | 558.4 | 4.0 |
| 39.3 | 0.1 | 11.0 | 2.3 | 101.0 | 0.7 |
| 40.9 | 0.1 | 13.5 | 2.2 | 123.9 | 0.9 |
| 41.3 | 0.1 | 10.5 | 2.2 | 64.1 | 0.5 |
| 41.6 | 0.2 | 13.7 | 2.2 | 63.0 | 0.5 |
| 43.4 | 0.1 | 22.4 | 2.1 | 205.8 | 1.5 |
| 45.1 | 0.1 | 13.6 | 2.0 | 125.1 | 0.9 |
| 45.5 | 0.2 | 9.5 | 2.0 | 34.8 | 0.3 |
| 45.9 | 0.1 | 23.7 | 2.0 | 174.3 | 1.3 |
| 46.5 | 0.1 | 9.0 | 2.0 | 82.8 | 0.6 |
| 48.0 | 0.1 | 31.5 | 1.9 | 289.5 | 2.1 |
| 49.0 | 0.1 | 15.2 | 1.9 | 93.1 | 0.7 |
| 49.4 | 0.1 | 14.1 | 1.8 | 86.3 | 0.6 |
| 50.8 | 0.2 | 10.8 | 1.8 | 33.0 | 0.2 |
| 51.5 | 0.1 | 13.6 | 1.8 | 124.9 | 0.9 |
| 52.3 | 0.1 | 10.8 | 1.7 | 99.0 | 0.7 |
| 52.5 | 0.1 | 13.7 | 1.7 | 126.1 | 0.9 |
| 52.9 | 0.1 | 72.9 | 1.7 | 446.8 | 3.2 |
| 53.9 | 0.1 | 9.2 | 1.7 | 112.4 | 0.8 |
| 56.3 | 0.1 | 6.4 | 1.6 | 33.5 | 0.2 |
| 57.3 | 0.1 | 9.1 | 1.6 | 55.7 | 0.4 |
| 58.6 | 0.1 | 17.9 | 1.6 | 109.4 | 0.8 |
| 60.3 | 0.2 | 8.9 | 1.5 | 27.3 | 0.2 |
| 62.4 | 0.4 | 16.0 | 1.5 | 29.4 | 0.2 |
| 63.9 | 0.2 | 5.7 | 1.5 | 17.6 | 0.1 |
| 64.4 | 0.1 | 5.9 | 1.4 | 36.1 | 0.3 |

A DSC thermogram for Compound 1 Phosphate is shown in FIG. 8 (FIG. 8). The thermogram is characterized by an endotherm peak at a temperature of about 213° C. FIG. 9 (FIG. 9) shows a thermogravimetric analysis (TGA) thermogram of Compound 1 Phosphate. FIG. 10 (FIG. 10) shows a dynamic vapor sorption (DVS) isotherm of Compound 1 Phosphate.

### Example 6. Compound 1 L-Tartrate Form LA

The XRPD spectrum for Compound 1 Form LA is provided in FIG. 11 (FIG. 11) and the corresponding peak data is provided below in Table 5.

TABLE 5
XRPD Peak Data for Compound 1 L-Tartrate Form LA

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 4.7 | 0.5 | 15.1 | 18.8 | 23.2 | 0.1 |
| 5.1 | 0.2 | 7.5 | 17.3 | 27.6 | 0.1 |
| 6.1 | 0.2 | 91.6 | 14.6 | 336.6 | 1.2 |
| 9.1 | 0.2 | 55.6 | 9.8 | 255.4 | 0.9 |
| 12.1 | 0.2 | 570.4 | 7.3 | 2621.2 | 9.5 |
| 12.4 | 0.2 | 161.8 | 7.2 | 743.5 | 2.7 |
| 12.8 | 0.2 | 55.3 | 6.9 | 203.4 | 0.7 |
| 13.4 | 0.2 | 65.3 | 6.6 | 240.0 | 0.9 |
| 14.1 | 0.2 | 85.6 | 6.3 | 314.8 | 1.1 |
| 15.0 | 0.2 | 286.0 | 5.9 | 1051.5 | 3.8 |
| 15.9 | 0.1 | 58.1 | 5.6 | 427.5 | 1.6 |
| 16.4 | 0.3 | 407.9 | 5.4 | 1071.2 | 3.9 |
| 16.9 | 0.1 | 170.1 | 5.2 | 1042.1 | 3.8 |
| 17.1 | 0.1 | 177.0 | 5.2 | 1301.4 | 4.7 |
| 18.1 | 0.2 | 7483.7 | 4.9 | 27513.5 | 100.0 |
| 19.3 | 0.1 | 182.5 | 4.6 | 958.4 | 3.5 |
| 19.9 | 0.1 | 96.3 | 4.5 | 590.0 | 2.1 |
| 20.5 | 0.3 | 192.8 | 4.3 | 506.3 | 1.8 |
| 21.1 | 0.1 | 23.5 | 4.2 | 144.1 | 0.5 |
| 21.7 | 0.2 | 172.9 | 4.1 | 635.8 | 2.3 |
| 22.1 | 0.2 | 74.1 | 4.0 | 340.4 | 1.2 |
| 22.5 | 0.2 | 34.9 | 3.9 | 160.3 | 0.6 |
| 23.0 | 0.2 | 144.9 | 3.9 | 443.8 | 1.6 |
| 23.9 | 0.2 | 910.0 | 3.7 | 4182.2 | 15.2 |
| 24.2 | 0.1 | 1554.5 | 3.7 | 11430.2 | 41.5 |
| 24.8 | 0.1 | 381.4 | 3.6 | 2003.3 | 7.3 |
| 25.8 | 0.2 | 92.7 | 3.5 | 426.0 | 1.6 |
| 26.2 | 0.1 | 60.4 | 3.4 | 370.1 | 1.4 |
| 27.0 | 0.1 | 361.4 | 3.3 | 1897.9 | 6.9 |
| 27.2 | 0.1 | 306.0 | 3.3 | 2249.8 | 8.2 |
| 27.6 | 0.2 | 162.1 | 3.2 | 744.8 | 2.7 |
| 28.4 | 0.1 | 220.1 | 3.1 | 1156.1 | 4.2 |
| 29.0 | 0.2 | 220.8 | 3.1 | 811.7 | 3.0 |
| 29.4 | 0.2 | 94.4 | 3.0 | 433.8 | 1.6 |
| 29.8 | 0.1 | 166.3 | 3.0 | 1019.1 | 3.7 |
| 30.3 | 0.1 | 236.2 | 2.9 | 1736.9 | 6.3 |
| 30.7 | 0.2 | 142.4 | 2.9 | 523.6 | 1.9 |
| 31.2 | 0.2 | 76.2 | 2.9 | 280.3 | 1.0 |
| 31.8 | 0.2 | 52.2 | 2.8 | 191.8 | 0.7 |
| 32.7 | 0.2 | 94.2 | 2.7 | 346.3 | 1.3 |
| 33.1 | 0.2 | 83.3 | 2.7 | 255.2 | 0.9 |
| 33.5 | 0.2 | 67.1 | 2.7 | 246.6 | 0.9 |
| 34.1 | 0.2 | 120.2 | 2.6 | 441.8 | 1.6 |
| 34.4 | 0.1 | 74.6 | 2.6 | 457.2 | 1.7 |
| 34.9 | 0.1 | 21.1 | 2.6 | 129.4 | 0.5 |
| 35.6 | 0.2 | 163.1 | 2.5 | 749.4 | 2.7 |
| 36.6 | 0.2 | 239.8 | 2.5 | 1101.8 | 4.0 |
| 37.1 | 0.1 | 171.4 | 2.4 | 900.0 | 3.3 |
| 38.3 | 0.1 | 63.9 | 2.3 | 391.6 | 1.4 |
| 38.6 | 0.1 | 134.4 | 2.3 | 823.8 | 3.0 |
| 39.0 | 0.2 | 50.3 | 2.3 | 154.1 | 0.6 |

A DSC thermogram for Form LA is shown in FIG. 12 (FIG. 12). The thermogram is characterized by endotherm peaks at temperatures of about 89° C. and about 138° C. FIG. 13 (FIG. 13) shows a thermogravimetric analysis (TGA) thermogram of Compound 1 L-Tartrate Form LA. FIG. 14 (FIG. 14) shows a dynamic vapor sorption (DVS) isotherm of Form LA.

### Example 7. Compound 1 L-Tartrate Form LB

The XRPD spectrum for Compound 1 L-Tartrate Form LB is provided in FIG. 15 (FIG. 15) and the corresponding peak data is provided below in Table 6.

TABLE 6
XRPD Peak Data for Compound 1 L-Tartrate Form LB

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 4.3 | 0.5 | 9.5 | 20.7 | 14.6 | 0.0 |
| 6.3 | 0.1 | 269.5 | 14.1 | 1651.3 | 4.2 |
| 8.9 | 0.7 | 33.5 | 9.9 | 38.4 | 0.1 |
| 11.9 | 0.1 | 66.9 | 7.4 | 410.0 | 1.1 |
| 12.1 | 0.2 | 221.2 | 7.3 | 813.2 | 2.1 |
| 12.5 | 0.1 | 302.4 | 7.1 | 1853.2 | 4.7 |
| 12.8 | 0.1 | 60.1 | 6.9 | 368.0 | 0.9 |
| 13.4 | 0.2 | 19.1 | 6.6 | 58.5 | 0.2 |
| 14.1 | 0.1 | 8.5 | 6.3 | 51.9 | 0.1 |
| 14.9 | 0.2 | 82.8 | 5.9 | 253.6 | 0.7 |
| 15.5 | 0.1 | 222.5 | 5.7 | 1363.3 | 3.5 |
| 15.9 | 0.1 | 28.6 | 5.6 | 175.2 | 0.5 |
| 16.3 | 0.2 | 186.9 | 5.4 | 763.5 | 2.0 |
| 16.5 | 0.1 | 65.6 | 5.4 | 402.0 | 1.0 |
| 16.9 | 0.1 | 63.5 | 5.2 | 389.1 | 1.0 |
| 18.1 | 0.1 | 717.7 | 4.9 | 5276.8 | 13.5 |
| 18.7 | 0.1 | 6395.4 | 4.7 | 39187.7 | 100.0 |
| 19.2 | 0.2 | 34.3 | 4.6 | 157.5 | 0.4 |
| 19.6 | 0.1 | 65.8 | 4.5 | 403.0 | 1.0 |
| 19.9 | 0.1 | 146.2 | 4.5 | 1074.9 | 2.7 |
| 20.0 | 0.1 | 227.2 | 4.4 | 1391.9 | 3.6 |
| 20.5 | 0.2 | 30.5 | 4.3 | 93.6 | 0.2 |
| 22.0 | 0.2 | 38.5 | 4.0 | 118.1 | 0.3 |
| 22.5 | 0.1 | 59.3 | 4.0 | 435.9 | 1.1 |
| 22.6 | 0.1 | 50.8 | 3.9 | 373.3 | 1.0 |
| 23.9 | 0.2 | 459.3 | 3.7 | 2110.9 | 5.4 |
| 24.2 | 0.1 | 224.6 | 3.7 | 1651.5 | 4.2 |
| 24.6 | 0.1 | 154.1 | 3.6 | 1132.8 | 2.9 |
| 25.0 | 0.1 | 3586.6 | 3.6 | 21976.5 | 56.1 |
| 27.1 | 0.2 | 123.6 | 3.3 | 568.0 | 1.5 |
| 28.4 | 0.1 | 12.9 | 3.1 | 78.7 | 0.2 |
| 28.8 | 0.3 | 94.4 | 3.1 | 247.9 | 0.6 |
| 29.4 | 0.1 | 114.3 | 3.0 | 700.1 | 1.8 |
| 29.8 | 0.2 | 43.4 | 3.0 | 199.5 | 0.5 |
| 30.3 | 0.1 | 61.7 | 2.9 | 323.9 | 0.8 |
| 30.7 | 0.2 | 137.7 | 2.9 | 632.9 | 1.6 |
| 31.0 | 0.2 | 64.8 | 2.9 | 298.0 | 0.8 |
| 31.4 | 0.1 | 1318.4 | 2.8 | 8078.3 | 20.6 |
| 33.1 | 0.2 | 28.4 | 2.7 | 104.5 | 0.3 |
| 33.8 | 0.2 | 48.9 | 2.6 | 149.7 | 0.4 |
| 34.0 | 0.1 | 10.2 | 2.6 | 74.8 | 0.2 |
| 34.1 | 0.2 | 25.3 | 2.6 | 77.6 | 0.2 |
| 34.6 | 0.1 | 29.1 | 2.6 | 178.5 | 0.5 |
| 35.1 | 0.1 | 77.6 | 2.6 | 475.3 | 1.2 |
| 35.9 | 0.2 | 99.2 | 2.5 | 364.7 | 0.9 |
| 36.4 | 0.1 | 38.0 | 2.5 | 232.9 | 0.6 |
| 36.7 | 0.2 | 173.5 | 2.4 | 797.3 | 2.0 |
| 37.0 | 0.1 | 79.1 | 2.4 | 484.8 | 1.2 |
| 37.7 | 0.2 | 23.6 | 2.4 | 108.3 | 0.3 |
| 37.9 | 0.1 | 279.5 | 2.4 | 2055.0 | 5.2 |
| 38.9 | 0.2 | 1.6 | 2.3 | 5.8 | 0.0 |
| 39.6 | 0.1 | 18.1 | 2.3 | 110.8 | 0.3 |

FIG. 16 (FIG. 16) shows a dynamic vapor sorption (DVS) isotherm of Form LB.

### Example 8. Compound 1 L-Tartrate Form LC

The XRPD spectrum for Compound 1 L-Tartrate Form LC is provided in FIG. 17 (FIG. 17) and the corresponding peak data is provided below in Table 7.

TABLE 7
XRPD Peak Data for Compound 1 L-Tartrate Form LC

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 6.3 | 0.2 | 57.4 | 14.1 | 175.8 | 3.0 |
| 12.2 | 0.2 | 908.8 | 7.3 | 4176.6 | 70.7 |
| 12.8 | 0.2 | 389.8 | 6.9 | 1791.5 | 30.3 |
| 15.0 | 0.1 | 168.1 | 5.9 | 882.8 | 15.0 |
| 15.4 | 0.1 | 420.8 | 5.7 | 3093.9 | 52.4 |
| 16.5 | 0.2 | 1001.8 | 5.4 | 4092.4 | 69.3 |
| 18.7 | 0.2 | 825.8 | 4.7 | 3373.2 | 57.1 |
| 19.5 | 0.1 | 179.9 | 4.5 | 1102.1 | 18.7 |
| 19.8 | 0.1 | 646.7 | 4.5 | 3396.4 | 57.5 |
| 20.0 | 0.1 | 557.8 | 4.4 | 2929.7 | 49.6 |
| 22.4 | 0.1 | 290.2 | 4.0 | 1777.9 | 30.1 |
| 22.6 | 0.2 | 614.3 | 3.9 | 2822.9 | 47.8 |
| 23.7 | 0.3 | 107.2 | 3.8 | 246.2 | 4.2 |
| 24.2 | 0.3 | 211.4 | 3.7 | 555.1 | 9.4 |
| 24.8 | 0.1 | 963.6 | 3.6 | 5904.1 | 100.0 |
| 25.0 | 0.1 | 697.9 | 3.6 | 3665.6 | 62.1 |
| 25.5 | 0.2 | 1163.6 | 3.5 | 5347.3 | 90.6 |
| 26.2 | 0.2 | 97.8 | 3.4 | 449.6 | 7.6 |
| 26.5 | 0.1 | 99.7 | 3.4 | 611.0 | 10.4 |
| 27.1 | 0.1 | 356.3 | 3.3 | 1871.2 | 31.7 |
| 27.8 | 0.1 | 83.2 | 3.2 | 509.6 | 8.6 |
| 28.0 | 0.1 | 85.6 | 3.2 | 524.7 | 8.9 |
| 29.0 | 0.2 | 286.3 | 3.1 | 1315.9 | 22.3 |
| 29.4 | 0.1 | 102.3 | 3.0 | 752.4 | 12.7 |
| 29.9 | 0.2 | 259.2 | 3.0 | 1191.2 | 20.2 |
| 30.8 | 0.2 | 282.3 | 2.9 | 1037.7 | 17.6 |
| 31.4 | 0.1 | 199.3 | 2.8 | 1465.7 | 24.8 |
| 32.9 | 0.1 | 68.5 | 2.7 | 419.4 | 7.1 |
| 33.4 | 0.7 | 131.0 | 2.7 | 150.5 | 2.6 |
| 33.8 | 0.1 | 86.4 | 2.7 | 529.2 | 9.0 |
| 34.6 | 0.1 | 75.8 | 2.6 | 557.2 | 9.4 |
| 35.0 | 0.2 | 121.9 | 2.6 | 373.3 | 6.3 |
| 35.4 | 0.1 | 52.4 | 2.5 | 385.6 | 6.5 |
| 36.0 | 0.1 | 273.1 | 2.5 | 1673.6 | 28.4 |
| 36.6 | 0.1 | 104.7 | 2.5 | 641.3 | 10.9 |
| 37.0 | 0.1 | 139.8 | 2.4 | 856.8 | 14.5 |
| 37.6 | 0.2 | 277.9 | 2.4 | 1135.3 | 19.2 |
| 39.6 | 0.1 | 114.0 | 2.3 | 598.9 | 10.1 |

A DSC thermogram for Form LC is shown in FIG. 18 (FIG. 18). The thermogram is characterized by an endotherm peak at a temperature of about 137° C. FIG. 19 (FIG. 19) shows a thermogravimetric analysis (TGA) thermogram of Compound 1 L-Tartrate Form LC. FIG. 20 (FIG. 20) shows a dynamic vapor sorption (DVS) isotherm of Form LC.

### Example 9. Compound 1 D-Tartrate

The XRPD spectrum for Compound 1 D-Tartrate is provided in FIG. 22 (FIG. 22) and the corresponding peak data is provided below in Table 8.

TABLE 8
XRPD Peak Data for Compound 1 D-Tartrate

| | FWHM | | d- | Rel. |
|---|---|---|---|---|
| Pos. | Left | Area | spacing | Int. |
| [°2θ] | [°2θ] | [cts*°2θ] | [Å] | [%] |
| 6.0 | 0.2 | 71.0 | 14.8 | 4.0 |
| 11.9 | 0.1 | 286.4 | 7.4 | 25.7 |
| 12.3 | 0.1 | 129.2 | 7.2 | 11.6 |
| 13.4 | 0.2 | 115.0 | 6.6 | 6.5 |
| 14.9 | 0.1 | 57.0 | 5.9 | 4.3 |
| 16.1 | 0.1 | 120.8 | 5.5 | 9.1 |
| 16.9 | 0.1 | 432.0 | 5.3 | 32.4 |
| 17.9 | 0.1 | 1112.7 | 4.9 | 100.0 |
| 19.1 | 0.1 | 202.7 | 4.6 | 15.2 |
| 20.6 | 0.1 | 41.3 | 4.3 | 3.1 |
| 21.2 | 0.1 | 32.2 | 4.2 | 2.4 |
| 21.6 | 0.1 | 125.1 | 4.1 | 9.4 |
| 22.1 | 0.2 | 37.8 | 4.0 | 2.1 |
| 23.4 | 0.1 | 57.1 | 3.8 | 4.3 |
| 23.9 | 0.1 | 523.8 | 3.7 | 33.6 |
| 24.6 | 0.1 | 102.0 | 3.6 | 9.2 |
| 25.1 | 0.2 | 167.0 | 3.5 | 8.3 |
| 25.9 | 0.1 | 86.3 | 3.4 | 6.5 |
| 26.8 | 0.1 | 87.1 | 3.3 | 7.8 |
| 27.2 | 0.2 | 59.7 | 3.3 | 2.7 |
| 28.7 | 0.2 | 78.5 | 3.1 | 3.5 |
| 29.6 | 0.1 | 106.2 | 3.0 | 8.0 |
| 30.1 | 0.1 | 84.6 | 3.0 | 7.6 |
| 30.4 | 0.2 | 96.6 | 2.9 | 4.3 |
| 31.5 | 0.3 | 31.9 | 2.8 | 0.9 |
| 32.4 | 0.2 | 57.6 | 2.8 | 3.2 |
| 33.8 | 0.2 | 22.5 | 2.7 | 1.3 |
| 34.2 | 0.2 | 43.8 | 2.6 | 1.6 |
| 35.3 | 0.1 | 150.3 | 2.5 | 9.7 |
| 36.3 | 0.2 | 62.3 | 2.5 | 3.5 |
| 36.9 | 0.1 | 33.8 | 2.4 | 2.5 |
| 38.3 | 0.2 | 35.1 | 2.3 | 2.0 |
| 38.8 | 0.2 | 45.1 | 2.3 | 2.5 |

A DSC thermogram for Compound 1 D-Tartrate is shown in FIG. 23 (FIG. 23). The thermogram is characterized by endotherm peaks at temperatures of about 76° C. and about 153° C. FIG. 23 (FIG. 23) shows a thermogravimetric analysis (TGA) thermogram of Compound 1 D-Tartrate. FIG. 24 (FIG. 24) shows a dynamic vapor sorption (DVS) isotherm of Compound 1 D-Tartrate.

### Example 10. Compound 1 Fumarate Form FA

The XRPD spectrum for Compound 1 Fumarate FA is provided in FIG. 25 (FIG. 25) and the corresponding peak data is provided below in Table 9.

TABLE 9
XRPD Peak Data for Compound 1 Fumarate Form FA

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 5.0 | 0.3 | 7.1 | 17.7 | 16.4 | 0.0 |
| 7.7 | 0.2 | 2022.3 | 11.5 | 9293.7 | 21.3 |
| 11.3 | 0.2 | 8.6 | 7.8 | 26.3 | 0.1 |
| 12.1 | 0.2 | 161.3 | 7.3 | 741.3 | 1.7 |
| 13.0 | 0.1 | 429.8 | 6.8 | 2633.3 | 6.0 |
| 14.2 | 0.1 | 911.3 | 6.2 | 4786.2 | 11.0 |
| 14.6 | 0.1 | 339.9 | 6.0 | 1784.9 | 4.1 |
| 15.2 | 0.1 | 1404.9 | 5.8 | 10330.0 | 23.7 |
| 15.9 | 0.2 | 13.6 | 5.6 | 62.5 | 0.1 |
| 16.2 | 0.2 | 18.9 | 5.5 | 69.5 | 0.2 |
| 17.1 | 0.2 | 22.9 | 5.2 | 105.2 | 0.2 |
| 18.1 | 0.2 | 183.7 | 4.9 | 844.4 | 1.9 |
| 18.8 | 0.1 | 126.3 | 4.7 | 928.9 | 2.1 |
| 19.1 | 0.1 | 6.2 | 4.6 | 38.1 | 0.1 |
| 19.3 | 0.1 | 5.6 | 4.6 | 34.3 | 0.1 |
| 19.9 | 0.3 | 32.9 | 4.5 | 75.5 | 0.2 |
| 20.8 | 0.1 | 15.2 | 4.3 | 92.9 | 0.2 |
| 21.8 | 0.2 | 115.7 | 4.1 | 531.7 | 1.2 |
| 22.4 | 0.1 | 135.0 | 4.0 | 993.0 | 2.3 |
| 22.6 | 0.1 | 162.7 | 3.9 | 996.7 | 2.3 |
| 22.9 | 0.1 | 4216.3 | 3.9 | 25835.4 | 59.3 |
| 23.9 | 0.2 | 85.0 | 3.7 | 390.4 | 0.9 |
| 24.2 | 0.1 | 42.2 | 3.7 | 258.7 | 0.6 |
| 24.6 | 0.1 | 1727.3 | 3.6 | 10583.8 | 24.3 |
| 25.0 | 0.1 | 5.0 | 3.6 | 30.7 | 0.1 |
| 25.5 | 0.1 | 2.0 | 3.5 | 12.0 | 0.0 |
| 26.0 | 0.2 | 500.3 | 3.4 | 2299.2 | 5.3 |
| 26.5 | 0.2 | 193.7 | 3.4 | 890.0 | 2.0 |
| 27.0 | 0.1 | 61.1 | 3.3 | 374.5 | 0.9 |
| 27.2 | 0.1 | 16.6 | 3.3 | 101.5 | 0.2 |
| 27.6 | 0.2 | 55.7 | 3.2 | 170.6 | 0.4 |
| 28.2 | 0.1 | 22.4 | 3.2 | 164.6 | 0.4 |
| 28.6 | 0.1 | 25.1 | 3.1 | 153.9 | 0.4 |
| 29.0 | 0.1 | 27.7 | 3.1 | 203.7 | 0.5 |
| 29.3 | 0.2 | 74.8 | 3.0 | 343.9 | 0.8 |
| 30.1 | 0.1 | 4.5 | 3.0 | 33.2 | 0.1 |
| 30.7 | 0.1 | 7109.3 | 2.9 | 43561.8 | 100.0 |
| 31.6 | 0.2 | 441.0 | 2.8 | 2026.5 | 4.7 |
| 32.2 | 0.1 | 27.6 | 2.8 | 169.2 | 0.4 |
| 32.8 | 0.4 | 93.0 | 2.7 | 170.9 | 0.4 |
| 33.3 | 0.1 | 68.3 | 2.7 | 418.2 | 1.0 |
| 34.6 | 0.2 | 48.2 | 2.6 | 177.1 | 0.4 |
| 35.2 | 0.1 | 165.4 | 2.5 | 1013.5 | 2.3 |
| 35.9 | 0.2 | 20.1 | 2.5 | 74.0 | 0.2 |
| 37.0 | 0.1 | 35.8 | 2.4 | 263.0 | 0.6 |
| 37.9 | 0.1 | 175.8 | 2.4 | 1077.3 | 2.5 |
| 38.6 | 0.2 | 1019.2 | 2.3 | 4684.0 | 10.8 |
| 39.7 | 0.2 | 24.5 | 2.3 | 112.4 | 0.3 |

A DSC thermogram for Form FA is shown in FIG. 26 (FIG. 26). The thermogram is characterized by an endotherm peak at a temperature of about 147° C. FIG. 27 (FIG. 27) shows a thermogravimetric analysis (TGA) thermogram of Compound 1 Fumarate Form FA. FIG. 28 (FIG. 28) shows a dynamic vapor sorption (DVS) isotherm of Form FA.

### Example 11. Compound 1 Fumarate Form FB

The XRPD spectrum for Compound 1 Fumarate FB is provided in FIG. 29 (FIG. 29) and the corresponding peak data is provided below in Table 10.

TABLE 10
XRPD Peak Data for Compound 1 Fumarate Form FB

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 4.7 | 0.5 | 18.2 | 18.9 | 27.9 | 0.0 |
| 6.7 | 0.1 | 2225.1 | 13.1 | 13634.5 | 9.2 |
| 9.0 | 0.1 | 118.6 | 9.8 | 871.7 | 0.6 |
| 9.7 | 0.1 | 5.6 | 9.1 | 41.3 | 0.0 |
| 11.1 | 0.1 | 7.2 | 8.0 | 44.1 | 0.0 |
| 11.7 | 0.2 | 49.6 | 7.6 | 182.4 | 0.1 |
| 12.1 | 0.1 | 31.3 | 7.3 | 191.5 | 0.1 |
| 12.5 | 0.2 | 152.2 | 7.1 | 699.5 | 0.5 |
| 13.0 | 0.2 | 207.1 | 6.8 | 634.3 | 0.4 |
| 13.4 | 0.1 | 868.4 | 6.6 | 5321.2 | 3.6 |
| 13.8 | 0.1 | 3273.3 | 6.4 | 20057.1 | 13.6 |
| 14.7 | 0.1 | 30.2 | 6.0 | 185.3 | 0.1 |
| 15.9 | 0.1 | 7.8 | 5.6 | 48.0 | 0.0 |
| 16.2 | 0.1 | 52.0 | 5.5 | 318.6 | 0.2 |
| 17.8 | 0.2 | 506.6 | 5.0 | 2328.1 | 1.6 |
| 18.4 | 0.1 | 102.2 | 4.8 | 626.3 | 0.4 |
| 19.3 | 0.1 | 85.3 | 4.6 | 448.1 | 0.3 |
| 20.2 | 0.1 | 24120.6 | 4.4 | 147797.7 | 100.0 |
| 20.7 | 0.1 | 135.2 | 4.3 | 828.2 | 0.6 |
| 21.1 | 0.1 | 104.2 | 4.2 | 638.7 | 0.4 |
| 21.2 | 0.1 | 85.5 | 4.2 | 628.5 | 0.4 |
| 22.3 | 0.2 | 38.0 | 4.0 | 174.6 | 0.1 |
| 23.1 | 0.1 | 270.2 | 3.8 | 1655.6 | 1.1 |
| 23.5 | 0.1 | 1093.1 | 3.8 | 8037.4 | 5.4 |
| 24.2 | 0.1 | 316.6 | 3.7 | 2328.0 | 1.6 |
| 25.1 | 0.1 | 1032.5 | 3.5 | 7592.1 | 5.1 |
| 26.1 | 0.1 | 85.6 | 3.4 | 629.7 | 0.4 |
| 26.3 | 0.1 | 246.2 | 3.4 | 1508.5 | 1.0 |
| 27.0 | 0.1 | 22016.7 | 3.3 | 134906.2 | 91.3 |
| 27.7 | 0.2 | 408.1 | 3.2 | 1250.3 | 0.9 |
| 28.6 | 0.2 | 73.0 | 3.1 | 335.3 | 0.2 |
| 29.2 | 0.2 | 47.6 | 3.1 | 218.9 | 0.2 |
| 29.6 | 0.1 | 1056.9 | 3.0 | 6476.2 | 4.4 |
| 30.2 | 0.1 | 28.5 | 3.0 | 174.7 | 0.1 |
| 31.0 | 0.1 | 102.4 | 2.9 | 537.6 | 0.4 |
| 31.7 | 0.1 | 256.7 | 2.8 | 1573.0 | 1.1 |
| 32.6 | 0.1 | 24.5 | 2.7 | 150.0 | 0.1 |
| 32.8 | 0.1 | 122.3 | 2.7 | 749.7 | 0.5 |
| 33.1 | 0.1 | 111.4 | 2.7 | 819.1 | 0.6 |
| 33.9 | 0.1 | 395.1 | 2.6 | 2905.2 | 2.0 |
| 34.4 | 0.1 | 257.4 | 2.6 | 1893.0 | 1.3 |
| 35.0 | 0.2 | 25.8 | 2.6 | 95.0 | 0.1 |
| 35.3 | 0.1 | 37.1 | 2.5 | 194.8 | 0.1 |
| 35.6 | 0.2 | 95.5 | 2.5 | 438.8 | 0.3 |
| 36.1 | 0.1 | 112.4 | 2.5 | 688.5 | 0.5 |
| 36.5 | 0.2 | 168.2 | 2.5 | 773.1 | 0.5 |
| 36.8 | 0.2 | 107.5 | 2.4 | 493.8 | 0.3 |
| 37.4 | 0.2 | 58.1 | 2.4 | 213.8 | 0.1 |
| 37.9 | 0.1 | 132.2 | 2.4 | 809.9 | 0.6 |
| 38.6 | 0.2 | 42.2 | 2.3 | 129.3 | 0.1 |
| 39.0 | 0.1 | 60.0 | 2.3 | 367.4 | 0.3 |

A DSC thermogram for Form FB is shown in FIG. 30 (FIG. 30). The thermogram is characterized by endotherm peaks at temperatures of about 96° C., about 139° C., and about 146° C. FIG. 31 (FIG. 31) shows a thermogravimetric analysis (TGA) thermogram of Compound 1 Fumarate Form FB. FIG. 32 (FIG. 32) shows a dynamic vapor sorption (DVS) isotherm of Form FB.

### Example 12. Compound 1 Citrate

The XRPD spectrum for Compound 1 Citrate is provided in FIG. 33 (FIG. 33) and the corresponding peak data is provided below in Table 11.

TABLE 11
XRPD Peak Data for Compound 1 Citrate

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 6.5 | 0.1 | 638.9 | 13.6 | 3915.0 | 91.1 |
| 10.2 | 0.1 | 70.4 | 8.7 | 517.3 | 12.0 |
| 11.5 | 0.2 | 41.4 | 7.7 | 126.8 | 3.0 |
| 13.0 | 0.2 | 226.0 | 6.8 | 1038.5 | 24.2 |
| 13.9 | 0.2 | 43.2 | 6.4 | 198.5 | 4.6 |
| 14.5 | 0.1 | 160.4 | 6.1 | 1179.6 | 27.5 |
| 15.5 | 0.2 | 1052.1 | 5.7 | 4297.6 | 100.0 |
| 16.5 | 0.1 | 152.9 | 5.4 | 936.6 | 21.8 |
| 17.3 | 0.1 | 272.8 | 5.1 | 1671.6 | 38.9 |
| 17.8 | 0.1 | 414.1 | 5.0 | 3044.9 | 70.9 |
| 18.9 | 0.1 | 103.1 | 4.7 | 541.2 | 12.6 |
| 19.4 | 0.1 | 472.0 | 4.6 | 3470.7 | 80.8 |
| 20.4 | 0.1 | 778.0 | 4.3 | 4086.1 | 95.1 |
| 20.8 | 0.1 | 226.2 | 4.3 | 1385.8 | 32.3 |
| 21.2 | 0.1 | 511.2 | 4.2 | 3132.6 | 72.9 |
| 21.5 | 0.2 | 290.8 | 4.1 | 1336.5 | 31.1 |
| 22.0 | 0.2 | 416.0 | 4.0 | 1699.3 | 39.5 |
| 23.1 | 0.1 | 249.5 | 3.9 | 1528.8 | 35.6 |
| 24.3 | 0.2 | 85.8 | 3.7 | 394.4 | 9.2 |
| 24.6 | 0.1 | 87.3 | 3.6 | 641.8 | 14.9 |
| 25.1 | 0.1 | 161.9 | 3.5 | 992.2 | 23.1 |
| 26.0 | 0.1 | 569.5 | 3.4 | 3489.4 | 81.2 |
| 27.0 | 0.1 | 39.3 | 3.3 | 241.0 | 5.6 |
| 27.2 | 0.1 | 40.4 | 3.3 | 247.3 | 5.8 |
| 27.5 | 0.1 | 195.6 | 3.2 | 1198.4 | 27.9 |
| 27.9 | 0.1 | 85.5 | 3.2 | 523.6 | 12.2 |
| 28.5 | 0.2 | 91.5 | 3.1 | 420.3 | 9.8 |
| 29.2 | 0.1 | 184.4 | 3.1 | 1129.8 | 26.3 |
| 30.4 | 0.2 | 90.1 | 2.9 | 413.9 | 9.6 |
| 30.8 | 0.2 | 47.3 | 2.9 | 217.4 | 5.1 |
| 31.4 | 0.1 | 124.7 | 2.8 | 763.9 | 17.8 |
| 32.5 | 0.1 | 56.1 | 2.8 | 412.5 | 9.6 |
| 33.3 | 0.1 | 211.8 | 2.7 | 1297.9 | 30.2 |
| 33.8 | 0.1 | 46.2 | 2.7 | 283.3 | 6.6 |
| 34.8 | 0.2 | 91.0 | 2.6 | 278.6 | 6.5 |
| 35.3 | 0.1 | 138.9 | 2.5 | 850.8 | 19.8 |
| 36.5 | 0.1 | 136.9 | 2.5 | 1006.8 | 23.4 |
| 36.8 | 0.1 | 89.2 | 2.4 | 656.2 | 15.3 |
| 37.4 | 0.2 | 118.6 | 2.4 | 435.9 | 10.1 |
| 38.3 | 0.1 | 50.5 | 2.3 | 309.5 | 7.2 |

A DSC thermogram for Compound 1 Citrate is shown in FIG. 34 (FIG. 34). The thermogram is characterized by an endotherm peak at a temperature of about 142′° C. FIG. 35 (FIG. 35) shows a thermogravimetric analysis (TGA) thermogram of Compound 1 Citrate. FIG. 36 (FIG. 36) shows a dynamic vapor sorption (DVS) isotherm of Compound 1 Citrate.

### Example 13. Compound 1 Succinate

The XRPD spectrum for Compound 1 Succinate is provided in FIG. 37 (FIG. 37) and the corresponding peak data is provided below in Table 12.

TABLE 12
XRPD Peak Data for Compound 1 Succinate

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 6.6 | 0.1 | 78.4 | 13.3 | 576.5 | 11.0 |
| 9.0 | 0.2 | 59.6 | 9.8 | 273.7 | 5.2 |
| 11.5 | 0.1 | 79.6 | 7.7 | 487.5 | 9.3 |
| 12.8 | 0.1 | 223.3 | 6.9 | 1641.8 | 31.4 |
| 13.9 | 0.1 | 690.8 | 6.4 | 5079.1 | 97.2 |
| 14.2 | 0.1 | 47.7 | 6.2 | 292.3 | 5.6 |
| 16.0 | 0.1 | 81.8 | 5.5 | 501.4 | 9.6 |
| 16.4 | 0.1 | 40.7 | 5.4 | 249.1 | 4.8 |
| 16.8 | 0.1 | 40.4 | 5.3 | 247.3 | 4.7 |
| 17.2 | 0.1 | 35.5 | 5.1 | 217.3 | 4.2 |
| 17.7 | 0.2 | 87.6 | 5.0 | 268.5 | 5.1 |
| 18.3 | 0.1 | 39.8 | 4.9 | 243.6 | 4.7 |
| 19.2 | 0.1 | 79.4 | 4.6 | 417.1 | 8.0 |
| 19.8 | 0.1 | 852.5 | 4.5 | 5223.5 | 100.0 |
| 21.1 | 0.1 | 97.2 | 4.2 | 595.9 | 11.4 |
| 22.9 | 0.1 | 343.9 | 3.9 | 2528.7 | 48.4 |
| 23.3 | 0.1 | 304.5 | 3.8 | 1599.1 | 30.6 |
| 23.8 | 0.1 | 80.9 | 3.7 | 594.6 | 11.4 |
| 24.5 | 0.1 | 73.2 | 3.6 | 448.7 | 8.6 |
| 25.0 | 0.1 | 148.6 | 3.6 | 910.6 | 17.4 |
| 25.2 | 0.1 | 177.8 | 3.5 | 1307.4 | 25.0 |
| 25.4 | 0.1 | 340.4 | 3.5 | 1787.6 | 34.2 |
| 25.9 | 0.1 | 50.0 | 3.4 | 306.3 | 5.9 |
| 26.1 | 0.1 | 84.1 | 3.4 | 618.3 | 11.8 |
| 26.5 | 0.1 | 913.2 | 3.4 | 4796.2 | 91.8 |
| 27.2 | 0.2 | 118.0 | 3.3 | 542.4 | 10.4 |
| 28.3 | 0.1 | 69.6 | 3.1 | 426.7 | 8.2 |
| 29.3 | 0.1 | 184.3 | 3.0 | 1129.3 | 21.6 |
| 30.4 | 0.2 | 50.7 | 2.9 | 155.2 | 3.0 |
| 31.7 | 0.1 | 45.2 | 2.8 | 277.0 | 5.3 |
| 32.3 | 0.2 | 51.0 | 2.8 | 234.3 | 4.5 |
| 33.2 | 0.1 | 53.2 | 2.7 | 325.9 | 6.2 |
| 33.4 | 0.1 | 47.8 | 2.7 | 293.1 | 5.6 |
| 33.9 | 0.2 | 40.5 | 2.6 | 186.3 | 3.6 |
| 34.8 | 0.2 | 52.5 | 2.6 | 160.9 | 3.1 |
| 35.8 | 0.2 | 61.6 | 2.5 | 188.8 | 3.6 |
| 36.5 | 0.2 | 62.0 | 2.5 | 285.0 | 5.5 |
| 37.4 | 0.2 | 81.7 | 2.4 | 250.3 | 4.8 |
| 38.9 | 0.1 | 43.8 | 2.3 | 268.1 | 5.1 |
| 39.6 | 0.2 | 51.2 | 2.3 | 235.1 | 4.5 |

A DSC thermogram for Compound 1 Succinate is shown in FIG. 38 (FIG. 38). The thermogram is characterized by an endotherm peak at a temperature of about 153° C. FIG. 39 (FIG. 39) shows a thermogravimetric analysis (TGA) thermogram of Compound 1 Succinate. FIG. 40 (FIG. 40) shows a dynamic vapor sorption (DVS) isotherm of Compound 1 Succinate.

### Example 14. Compound 1 Glutarate

The XRPD spectrum for Compound 1 Glutarate is provided in FIG. 41 (FIG. 41) and the corresponding peak data is provided below in Table 13.

TABLE 13
XRPD Peak Data for Compound 1 Glutarate

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 9.1 | 0.1 | 487.6 | 9.7 | 3585.4 | 21.0 |
| 9.7 | 0.2 | 4.3 | 9.1 | 13.1 | 0.1 |
| 10.6 | 0.1 | 409.4 | 8.4 | 2150.4 | 12.6 |
| 11.3 | 0.2 | 7.3 | 7.8 | 33.6 | 0.2 |
| 11.9 | 0.2 | 28.2 | 7.4 | 129.6 | 0.8 |
| 12.7 | 0.1 | 7.5 | 7.0 | 55.1 | 0.3 |
| 14.2 | 0.1 | 37.5 | 6.2 | 230.0 | 1.4 |
| 15.0 | 0.2 | 31.0 | 5.9 | 142.4 | 0.8 |
| 15.9 | 0.1 | 74.0 | 5.6 | 388.5 | 2.3 |
| 16.8 | 0.1 | 85.0 | 5.3 | 520.6 | 3.1 |
| 18.2 | 0.1 | 2781.0 | 4.9 | 17040.5 | 100.0 |
| 18.6 | 0.1 | 18.4 | 4.8 | 134.9 | 0.8 |
| 19.0 | 0.1 | 503.2 | 4.7 | 3700.2 | 21.7 |
| 19.6 | 0.1 | 96.8 | 4.5 | 593.4 | 3.5 |
| 20.8 | 0.1 | 33.4 | 4.3 | 204.5 | 1.2 |
| 21.8 | 0.1 | 110.8 | 4.1 | 678.8 | 4.0 |
| 21.9 | 0.1 | 136.5 | 4.1 | 836.7 | 4.9 |
| 22.5 | 0.1 | 209.4 | 4.0 | 1539.6 | 9.0 |
| 23.1 | 0.2 | 58.2 | 3.8 | 214.0 | 1.3 |
| 23.6 | 0.1 | 17.4 | 3.8 | 106.3 | 0.6 |
| 25.0 | 0.2 | 97.3 | 3.6 | 446.9 | 2.6 |
| 25.3 | 0.1 | 39.1 | 3.5 | 239.3 | 1.4 |
| 25.8 | 0.1 | 135.9 | 3.4 | 999.2 | 5.9 |
| 26.4 | 0.1 | 74.2 | 3.4 | 545.5 | 3.2 |
| 27.4 | 0.1 | 368.3 | 3.2 | 2256.9 | 13.2 |
| 28.0 | 0.1 | 451.6 | 3.2 | 2767.2 | 16.2 |
| 28.5 | 0.1 | 25.9 | 3.1 | 190.2 | 1.1 |
| 29.5 | 0.1 | 19.8 | 3.0 | 121.5 | 0.7 |
| 30.0 | 0.1 | 56.1 | 3.0 | 412.6 | 2.4 |
| 30.3 | 0.1 | 10.1 | 3.0 | 61.6 | 0.4 |
| 31.5 | 0.2 | 27.9 | 2.8 | 85.5 | 0.5 |
| 32.9 | 0.2 | 40.1 | 2.7 | 123.0 | 0.7 |
| 33.8 | 0.1 | 32.2 | 2.6 | 197.0 | 1.2 |
| 35.2 | 0.1 | 42.2 | 2.6 | 258.5 | 1.5 |
| 35.6 | 0.2 | 26.2 | 2.5 | 80.1 | 0.5 |
| 36.8 | 0.1 | 148.0 | 2.4 | 1088.0 | 6.4 |
| 37.2 | 0.2 | 65.1 | 2.4 | 299.2 | 1.8 |
| 37.9 | 0.1 | 33.6 | 2.4 | 246.9 | 1.5 |
| 38.5 | 0.1 | 59.6 | 2.3 | 365.3 | 2.1 |
| 39.5 | 0.1 | 8.8 | 2.3 | 54.1 | 0.3 |

FIG. 42 (FIG. 42) shows a dynamic vapor sorption (DVS) isotherm of Compound 1 Glutarate.

### Example 15. Compound 1 L-Malate

The XRPD spectrum for Compound 1 L-Malate is provided in FIG. 43 (FIG. 43) and the corresponding peak data is provided below in Table 14.

TABLE 14
XRPD Peak Data for Compound 1 L-Malate

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 11.9 | 0.1 | 37.7 | 7.4 | 231.0 | 2.6 |
| 13.1 | 0.1 | 93.8 | 6.8 | 689.9 | 7.8 |
| 13.5 | 0.2 | 1102.0 | 6.5 | 5064.2 | 57.0 |
| 14.4 | 0.1 | 97.7 | 6.2 | 718.5 | 8.1 |
| 15.2 | 0.2 | 171.4 | 5.8 | 787.6 | 8.9 |
| 16.0 | 0.1 | 59.5 | 5.5 | 312.4 | 3.5 |
| 16.6 | 0.1 | 58.5 | 5.3 | 429.9 | 4.8 |
| 17.0 | 0.1 | 69.6 | 5.2 | 512.0 | 5.8 |
| 17.7 | 0.1 | 121.5 | 5.0 | 744.3 | 8.4 |
| 18.1 | 0.1 | 73.3 | 4.9 | 449.4 | 5.1 |
| 18.8 | 0.2 | 1935.1 | 4.7 | 8893.1 | 100.0 |
| 19.8 | 0.1 | 49.1 | 4.5 | 300.6 | 3.4 |
| 20.9 | 0.1 | 43.5 | 4.3 | 320.2 | 3.6 |
| 22.4 | 0.2 | 179.9 | 4.0 | 826.6 | 9.3 |
| 22.9 | 0.2 | 105.8 | 3.9 | 486.1 | 5.5 |
| 23.8 | 0.1 | 539.5 | 3.7 | 2833.7 | 31.9 |
| 24.6 | 0.1 | 138.8 | 3.6 | 1020.8 | 11.5 |
| 24.8 | 0.2 | 856.5 | 3.6 | 3148.9 | 35.4 |
| 25.2 | 0.2 | 1082.7 | 3.5 | 4422.7 | 49.7 |
| 25.6 | 0.1 | 241.8 | 3.5 | 1269.7 | 14.3 |
| 26.2 | 0.1 | 217.6 | 3.4 | 1142.8 | 12.9 |
| 26.8 | 0.2 | 89.7 | 3.3 | 274.7 | 3.1 |
| 27.2 | 0.1 | 61.3 | 3.3 | 450.6 | 5.1 |
| 27.7 | 0.1 | 134.8 | 3.2 | 826.1 | 9.3 |
| 28.0 | 0.2 | 105.8 | 3.2 | 486.4 | 5.5 |
| 28.6 | 0.2 | 75.7 | 3.1 | 347.8 | 3.9 |
| 28.9 | 0.1 | 39.8 | 3.1 | 244.1 | 2.7 |
| 29.4 | 0.4 | 74.8 | 3.0 | 137.4 | 1.6 |
| 30.5 | 0.1 | 104.3 | 2.9 | 767.2 | 8.6 |
| 32.3 | 0.1 | 36.3 | 2.8 | 222.7 | 2.5 |
| 33.1 | 0.2 | 75.5 | 2.7 | 277.5 | 3.1 |
| 33.4 | 0.2 | 74.1 | 2.7 | 272.6 | 3.1 |
| 34.4 | 0.2 | 58.7 | 2.6 | 269.7 | 3.0 |
| 34.8 | 0.3 | 51.2 | 2.6 | 117.6 | 1.3 |
| 35.4 | 0.2 | 63.3 | 2.5 | 194.1 | 2.2 |
| 35.9 | 0.2 | 123.8 | 2.5 | 568.9 | 6.4 |
| 37.1 | 0.3 | 124.8 | 2.4 | 286.8 | 3.2 |
| 37.5 | 0.3 | 49.2 | 2.4 | 113.1 | 1.3 |
| 38.2 | 0.3 | 157.5 | 2.4 | 361.9 | 4.1 |
| 39.5 | 0.2 | 18.1 | 2.3 | 83.2 | 0.9 |

A DSC thermogram for Compound 1 L-Malate is shown in FIG. 44 (FIG. 44). The thermogram is characterized by an endotherm peak at a temperature of about 82° C. FIG. 45 (FIG. 45) shows a thermogravimetric analysis (TGA) thermogram of Compound 1 L-Malate. FIG. 46 (FIG. 46) shows a dynamic vapor sorption (DVS) isotherm of Compound 1 L-Malate.

### Example 16. Compound 1 Besylate

The XRPD spectrum for Compound 1 Besylate is provided in FIG. 47 (FIG. 47) and the corresponding peak data is provided below in Table 15.

TABLE 15
XRPD Peak Data for Compound 1 Besylate

| | FWHM | | d- | | Rel. |
|---|---|---|---|---|---|
| Pos. | Left | Area | spacing | Height | Int. |
| [°2θ] | [°2θ] | [cts*°2θ] | [Å] | [cts] | [%] |
| 6.0 | 0.1 | 9260.8 | 14.6 | 56745.2 | 100.0 |
| 12.0 | 0.2 | 3664.6 | 7.4 | 16841.2 | 29.7 |
| 13.0 | 0.2 | 42.2 | 6.8 | 155.1 | 0.3 |
| 13.8 | 0.2 | 58.7 | 6.4 | 179.7 | 0.3 |
| 14.7 | 0.2 | 67.1 | 6.0 | 246.6 | 0.4 |
| 16.4 | 0.1 | 90.9 | 5.4 | 668.0 | 1.2 |
| 16.6 | 0.2 | 711.3 | 5.3 | 3269.0 | 5.8 |
| 16.9 | 0.1 | 54.1 | 5.2 | 331.6 | 0.6 |
| 18.1 | 0.1 | 73.6 | 4.9 | 451.0 | 0.8 |
| 18.6 | 0.1 | 81.6 | 4.8 | 600.1 | 1.1 |
| 19.0 | 0.1 | 102.1 | 4.7 | 750.5 | 1.3 |
| 19.4 | 0.1 | 70.5 | 4.6 | 518.7 | 0.9 |
| 20.3 | 0.2 | 91.9 | 4.4 | 281.6 | 0.5 |
| 21.2 | 0.1 | 101.0 | 4.2 | 742.6 | 1.3 |
| 21.4 | 0.1 | 94.2 | 4.1 | 692.3 | 1.2 |
| 22.2 | 0.1 | 126.7 | 4.0 | 931.6 | 1.6 |
| 23.2 | 0.1 | 160.2 | 3.8 | 1177.8 | 2.1 |
| 24.1 | 0.1 | 7880.7 | 3.7 | 48288.4 | 85.1 |
| 24.9 | 0.2 | 54.1 | 3.6 | 165.6 | 0.3 |
| 25.5 | 0.2 | 77.2 | 3.5 | 236.6 | 0.4 |
| 26.0 | 0.2 | 58.2 | 3.4 | 267.6 | 0.5 |
| 26.8 | 0.1 | 450.2 | 3.3 | 3310.2 | 5.8 |
| 28.6 | 0.2 | 31.5 | 3.1 | 115.8 | 0.2 |
| 29.6 | 0.2 | 24.0 | 3.0 | 110.1 | 0.2 |
| 30.3 | 0.1 | 1661.3 | 2.9 | 10179.2 | 17.9 |
| 31.9 | 0.2 | 174.8 | 2.8 | 803.5 | 1.4 |
| 34.0 | 0.1 | 47.0 | 2.6 | 345.3 | 0.6 |
| 34.6 | 0.2 | 26.0 | 2.6 | 119.4 | 0.2 |
| 35.5 | 0.1 | 52.8 | 2.5 | 323.7 | 0.6 |
| 36.0 | 0.1 | 67.0 | 2.5 | 410.6 | 0.7 |
| 36.6 | 0.2 | 108.9 | 2.5 | 333.6 | 0.6 |
| 37.4 | 0.1 | 119.7 | 2.4 | 880.4 | 1.6 |
| 38.4 | 0.1 | 107.9 | 2.3 | 661.2 | 1.2 |

A DSC thermogram for Compound 1 Besylate is shown in FIG. 48 (FIG. 48). The thermogram is characterized by an endotherm peak at a temperature of about 136° C. FIG. 49 (FIG. 49) shows a thermogravimetric analysis (TGA) thermogram of Compound 1 Besylate. FIG. 50 (FIG. 50) shows a dynamic vapor sorption (DVS) isotherm of Compound 1 Besylate.

### Example 17. Stability Study

Each sample was stored at 60° C. and 60° C. 75% RH for 1 month. Each sample was measured at 20 days and 1 month by HPLC. HPLC conditions are described below.
Column: Waters XSelect CSH C18, 4.6×150 mm, 3.5 μm
MPA: 50 mM HClO4 in water (pH=3)
MPB: MeOH/acetonitrile (9:1)
Wavelength: 215 nm
Column Temp: 40° C.
Flow: 1 mL/min

### Gradient:

| | |
|---|---|
| 0 min | 10% MPB |
| 12 min | 24% MPB |
| 20 min | 90% MPB |
| 20 min | 90% MPB |
| 21 min | 10% MPB |
| 31 min | 10% MPB |

The results of the stability are shown in Table 16 below.

TABLE 16
Stability Study Results

| | | | L- | Fumaric | Citric | Free- |
|---|---|---|---|---|---|---|
| | HCl | H₃PO₄ | Tartaric | acid | acid | base |
| Initial | 98.94 | 99.20 | 99.10 | 99.48 | 99.08 | 98.28 |
| 20 day at 60° C. | 98.50 | 99.20 | 99.13 | 99.54 | 99.10 | 76.57 |
| 1 month at 60° C. | 98.89 | 99.23 | 99.14 | 99.60 | 99.16 | 62.18 |
| 20 days at 60° C. | 99.07 | 99.20 | 99.23 | 99.67 | 98.34 | — |
| 75% RH | | | | | | |
| 1 month at 60° C. | 99.05 | 99.24 | 99.23 | 99.64 | 97.73 | 5.83 |
| 75% RH | | | | | | |

The freebase (amorphous) of Compound 1 was unstable at 60° C. and 60° C. 75% RH. The other salts (Hydrochloride, Phosphate, L-tartrate, Fumarate, citrate) were stable.

### Example 18. Phosphate Polymorph Screen

Compound 1 Phosphate was subjected to three forms of polymorph screening: solvent screening, slurry screening, and rapid cool screening. The procedures for each screening are provided below.

Solvent Screening: Compound 1 Phosphate (5 mg) was put into a small glass vial and solvents were added until the sample was dissolved at 90° C. or boiling point. Maximum volume of solvent was 500 μL. The solution or suspension was heated for 1 h, allowed to cool undisturbed at room temperature overnight, and cooled at 5° C. for 3 days. Un-precipitated samples were opened and allowed to stand at room temperature until dryness (slow evaporation). The precipitated solids were filtered by sintering filter plate, and measured directly by XRPD on the plate.

Slurry Screening: Compound 1 Phosphate (10 mg) was put into a small glass vial and organic solvents mixed with water were added. The samples were then suspended at room temperature or 50° C. and shaken for 4 to 10 days. The suspensions were filtered. The collected solids were analyzed by XRPD to determine polymorphic form.

Rapid Cool Screening: Compound 1 Phosphate (10 mg) was dissolved at 90° C. or boiling point. The solution was cooled at 0° C. rapidly. After 1 h, solution was cooled at −20° C. for 3 days. The precipitated crystals were filtered. Recovered solids were analyzed by XRPD to determine polymorphic form.

From the screening studies (over 75 trials), only one polymorph (Compound 1 Phosphate as described in Example 5) was identified.

### Example 19. Synthesis of Compound 1 and Compound 1 Phosphate

### Step 1. Preparation of 1-(5-bromo-8-fluoroisochroman-1-yl)-N-methylmethanamine trifluoromethanesulfonate

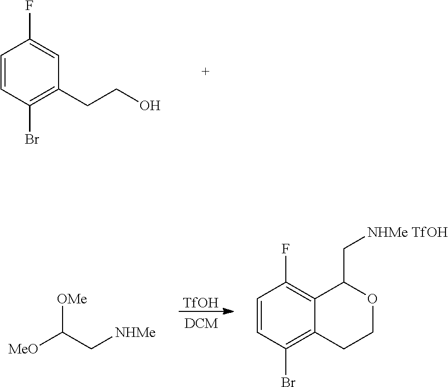

A solution of 2-(2-bromo-5-fluorophenyl)ethan-1-ol (200 g, 913 mmol) and N-methylaminoacetaldehyde dimethylacetal (130 g, 1091 mmol) in dichloromethane (300 mL) was cooled to 0° C. and trifluoromethanesulfonic acid (554 g, 3691 mmol) was added over 1 h while maintaining the temperature below 35° C. with external cooling. The mixture was warmed to 30° C. and stirred at 30° C. for 23 h. The mixture was cooled to 20° C. and methanol (33 mL), dichloromethane (1275 mL) and tert-butyl methyl ether (1358 mL) were added. The precipitate was collected by suction filtration. The solids were washed with a solution consisting of 1:1 (v/v) dichloromethane and tert-butyl methyl ether (1830 mL) and dried under vacuum (>28″ Hg) at 45° C. for 21 h to give 1-(5-bromo-8-fluoroisochroman-1-yl)-N-methylmethanamine trifluoromethanesulfonate (339 g, 87% yield) as a tan powder. MS (ESI): m/z 274, 276 [M+H]⁺; ¹H-NMR (400 MHz, DMSO-d₆): [ppm 8.65 (br s, 1H), 8.54 (br s, 1H), 7.67 (dd, J=8.80, 5.28 Hz, 1H), 7.15 (dd, J=9.98, 8.80 Hz, 1H), 5.21 (dd, J=9.78, 2.35 Hz, 1H), 4.06 (dt, J=12.03, 5.92 Hz, 1H), 3.89 (dt, J=11.93, 5.18 Hz, 1H), 3.50-3.38 (m, 1H), 3.31 (br s, 1H), 2.73 (t, J=5.67 Hz, 2H), 2.64 (br s, 3H); ¹³C-NMR (100 MHz, DMSO-d₆): [ppm 158.91, 156.48, 136.20, 136.16, 132.57, 132.48, 123.37, 123.20, 119.20, 119.17, 115.34, 115.10, 66.59, 59.63, 49.09, 49.04, 33.01, 28.75, 28.73.

### Step 2. Preparation of (R)-]-(8-fluoroisochroman-1-yl)-N-methylmethanaminium (2R,3R)-2,3-bis(benzoyloxy)-3-carboxypropanoate

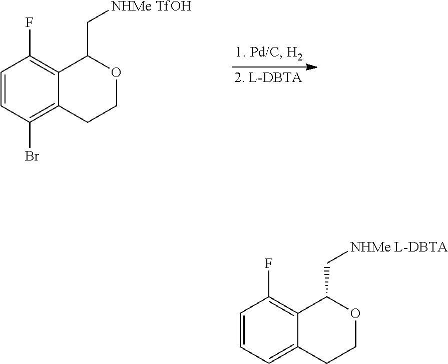

A solution of 1-(5-bromo-8-fluoroisochroman-1-yl)-N-methylmethanamine trifluoromethanesulfonate (300 g, 707 mmol), palladium, 10 wt. % (dry basis) on activated carbon, 50% water (1.2 g), methanol (1006 mL) and 5 wt. % aq. potassium carbonate (1059 g) was hydrogenated at 25° C., 5 bar hydrogen pressure, for 1 h. The pressure was released, and the solution was filtered through a 5-micron disposable polypropylene in-line filter cartridge which was rinsed with a solution consisting of 1:1 (v/v) methanol and water (540 g). The solution was concentrated under reduced pressure to a final volume of 1200 mL. To the mixture was added tert-butyl methyl ether (893 mL) and 20 wt. % aq. potassium hydroxide (185 mL). The mixture was stirred for 10 minutes at 20° C. The stirring was stopped and the phases were separated. The aqueous phase was extracted with tert-butyl methyl ether (649 mL). The combined organic layers were concentrated under reduced pressure to a final volume of 450 mL. To the solution was added SDA (specially denatured alcohol) 3A ethanol (851 mL). The solution was concentrated under reduced pressure to a final volume of 675 mL. The solution (solution A) was held for further processing. In a separate vessel was added dibenzoyl-L-tartaric acid (252 g, 703 mmol), SDA 3A ethanol (2704 mL) and deionized water (69 mL). The mixture was heated to approximately 70° C. and solution A was added over 9 minutes, which resulted in precipitation. The mixture was stirred at approximately 70° C. for 45 minutes then cooled to 20° C. over approximately 2.5 h. The precipitate was collected by suction filtration. The solids were washed with SDA 3A ethanol (960 mL) and dried under vacuum (>28″ Hg) at 45° C. for 17 h to give (R)-1-(8-fluoroisochroman-1-yl)-N-methylmethanaminium (2R,3R)-2,3-bis(benzoyloxy)-3-carboxypropanoate (181 g, 46% yield) as a white solid.

Alternative resolving agents, other than dibenzoyl-L-tartaric acid were explored, but most were found to not be suitable or practical for various reasons (e.g., availability, cost, performance). Such resolving agents include (R)-mandelic acid, L-tartaric acid, and L-malic acid. However, N-acetyl-D-leucine was found to provide the desired (R)-isomer in approximately 91:9 ratio and approximately 17% yield.

The optical purity of intermediate (R)-1-(8-fluoroisochroman-1-yl)-N-methylmethanaminium (2R,3R)-2,3-bis(benzoyloxy)-3-carboxypropanoate was enriched to 96.4% de by performing the following recrystallization procedure.

To (R)-1-(8-fluoroisochroman-1-yl)-N-methylmethanaminium (2R,3R)-2,3-bis(benzoyloxy)-3-carboxypropanoate (177 g, 320 mmol) was added methanol (3578 mL) and the solution was concentrated at atmospheric pressure to a final volume of 1326 mL which resulted in crystallization. The temperature of the slurry was adjusted to approximately 62° C. and stirring was continued for 20 min. The slurry was cooled to 10° C. over 2 h and the solids were collected by suction filtration. The solids were washed with cold (10° C.) methanol (675 mL) and dried under vacuum (>28″ Hg) at 45° C. overnight to give (R)-1-(8-fluoroisochroman-1-yl)-N-methylmethanaminium (2R,3R)-2,3-bis(benzoyloxy)-3-carboxypropanoate (136 g, 77% yield) as a white solid. MS (ESI): m/z 196 [M+H]⁺; ¹H-NMR (400 MHz, DMSO-d₆): [ppm 8.03-7.85 (m, 4H), 7.70-7.55 (m, 2H), 7.54-7.43 (m, 4H), 7.27 (td, J=7.92, 6.06 Hz, 1H), 7.11-6.89 (m, 2H), 5.67 (s, 2H), 5.15 (dd, J=9.98, 2.93 Hz, 1H), 3.92 (ddd, J=11.84, 7.34, 4.70 Hz, 1H), 3.69 (dt, J=11.54, 4.99 Hz, 1H), 3.36-3.24 (m, 1H), 3.22-3.04 (m, 1H), 2.80-2.61 (m, 2H), 2.54 (s, 3H); ¹³C-NMR (100 MHz, DMSO-d₆): [ppm 168.21, 164.89, 159.56, 157.13, 136.87, 136.82, 133.37, 129.60, 129.23, 128.88, 128.80, 128.65, 125.10, 125.07, 120.58, 120.43, 114.57, 113.07, 112.87, 72.47, 66.71, 59.65, 49.22, 49.17, 32.62, 27.28, 27.26.

### Step 3. Preparation of (R)-]-(8-fluoroisochroman-1-yl)-N-methylmethanamine phosphate

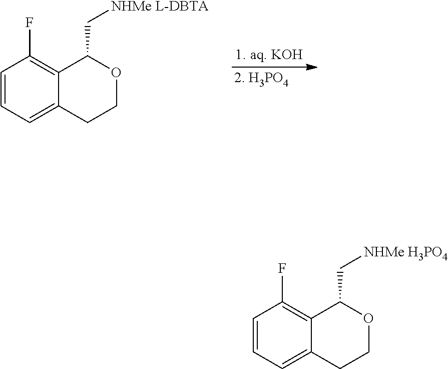

To a mixture of compound (R)-1-(8-fluoroisochroman-1-yl)-N-methylmethanaminium (2R,3R)-2,3-bis(benzoyloxy)-3-carboxypropanoate (75 g, 135.5 mmol) and tert-butyl methyl ether (223 mL) was added a solution consisting of 14 wt. % aq. potassium hydroxide (120 mL) and sodium chloride (15 g). The mixture was stirred for 10 minutes at 23-25° C. The stirring was stopped and the phases were separated. The aqueous phase was extracted with tert-butyl methyl ether (2×78 mL). The combined organic layers were concentrated under reduced pressure to a final volume of 90 mL. The solution was cooled to approximately 48° C. and acetonitrile (288 ml) was added. The solution was concentrated at atmospheric pressure to a final volume of approx. 210 mL. The mixture was cooled to 20° C. and acetonitrile (97 mL) and DI water (23 mL) were added. To this was added at 20° C. a solution consisting of 87% by weight aq. H₃PO₄ (16.9 g) and acetonitrile (81 mL). The product precipitated during the addition. The slurry was stirred for 3 h at 20° C. and the solids were collected by suction filtration. The solids were washed with a solution consisting of acetonitrile (135 mL) and DI water (8 mL) and dried under vacuum (>28″ Hg) at 55° C. overnight to give (R)-1-(8-fluoroisochroman-1-yl)-N-methylmethanamine phosphate (36 g, 89% yield) as a white crystalline solid, which is Compound 1 Phosphate as characterized in Example 5. MS (ESI): m/z 196 [M+H]⁺; ¹H-NMR (400 MHz, D₂O): [ppm 7.27 (td, J=7.83, 5.87 Hz, 1H), 7.03 (d, J=7.25 Hz, 1H), 6.97 (dd, J=10.37, 8.80 Hz, 1H), 5.25 (t, J=5.87 Hz, 1H), 4.04 (dt, J=11.74, 5.87 Hz, 1H), 3.91-3.80 (m, 1H), 3.47 (d, J=5.87 Hz, 2H); 2.83 (t, J=5.48 Hz, 2H), 2.75 (s, 3H); ¹³C-NMR (100 MHz, D₂O with 5% methanol-d₄): [ppm 161.09, 158.67, 138.10, 138.07, 130.51, 130.42, 126.19, 126.16, 120.28, 120.13, 114.37, 114.17, 68.33, 62.15, 51.39, 51.34, 34.05, 28.38, 28.35.

### Step 4. Recrystallization of (R)-]-(8-fluoroisochroman-1-yl)-N-methylmethanamine phosphate using water acetone

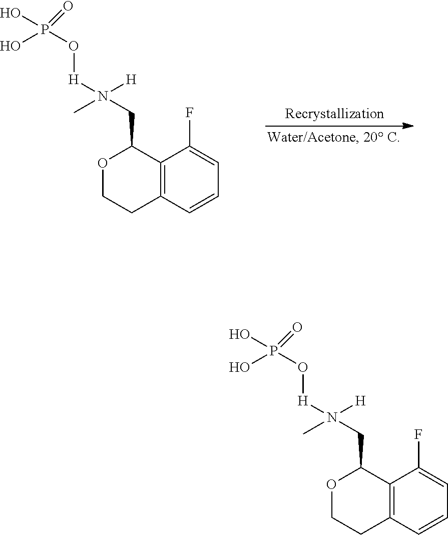

19.5 g of the product of (R)-1-(8-fluoroisochroman-1-yl)-N-methylmethanamine phosphate from step 3 was dissolved in 70 g water at 20° C., and then 30 g acetone was charged into the process stream to make the 16.3 wt % starting solution. The solution passed through the polish filter to remove any insoluble matters. Then dosing 600 g acetone into the solution within around 1 hour. The product crystallized out during the addition. The slurry was stirred at least 30 min at 20° C. and the solids were collected by suction filtration. The solids were washed with a binary solvents acetone (54 g) and DI water (6 g) and dried under vacuum (>28″ Hg) at 55° C. overnight to give (R)-1-(8-fluoroisochroman-1-yl)-N-methylmethanamine phosphate (18.1 g, 93% yield) as a white crystalline solid.

### Step 4. Recrystallization of (R)-1-(8-fluoroisochroman-1-yl)-N-methylmethanamine phosphate using water/acetonitrile

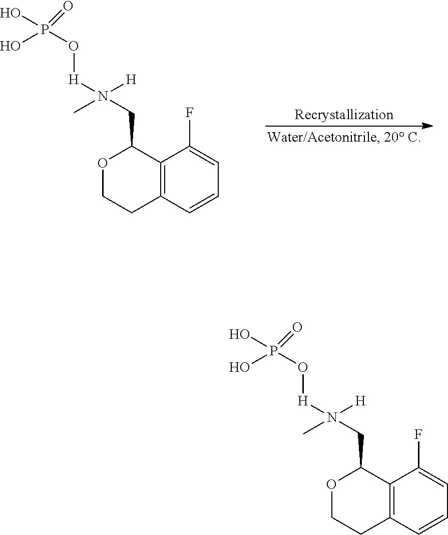

35.0 g of the product of (R)-1-(8-fluoroisochroman-1-yl)-N-methylmethanamine phosphate from step 3 was dissolved in 70 g water at 20° C., and then 30 g acetonitrile was charged into the process stream to make the 25.9 wt % starting solution. The solution passed through the polish filter to remove any insoluble matters. Then dosing 600 g acetonitrile into the solution within around 1 hour. The product crystallized out during the addition. The slurry was stirred at least 30 min at 20° C. and the solids were collected by suction filtration. The solids were washed with a binary solvents acetonitrile (90 g) and DI water (10 g) and dried under vacuum (>28″ Hg) at 55° C. overnight to give (R)-1-(8-fluoroisochroman-1-yl)-N-methylmethanamine phosphate (31.2 g, 89% yield) as a white crystalline solid.

Various preferred embodiments [A] to [DY] of the invention can be described in the text below:
[Embodiment A] A salt, which is:
(R)-1-(8-fluoroisochroman-1-yl)-N-methylmethanamine phosphate (Compound 1 Phosphate);
(R)-1-(8-fluoroisochroman-1-yl)-N-methylmethanamine L-tartrate (Compound 1 L-Tartrate);
(R)-1-(8-fluoroisochroman-1-yl)-N-methylmethanamine D-tartrate (Compound 1 D-Tartrate);
(R)-1-(8-fluoroisochroman-1-yl)-N-methylmethanamine fumarate (Compound 1 Fumarate);
(R)-1-(8-fluoroisochroman-1-yl)-N-methylmethanamine citrate (Compound 1 Citrate);
(R)-1-(8-fluoroisochroman-1-yl)-N-methylmethanamine succinate (Compound 1 Succinate);
(R)-1-(8-fluoroisochroman-1-yl)-N-methylmethanamine glutarate (Compound 1 Glutarate);
(R)-1-(8-fluoroisochroman-1-yl)-N-methylmethanamine L-malate (Compound 1 L-Malate);
(R)-1-(8-fluoroisochroman-1-yl)-N-methylmethanamine besylate (Compound 1 Besylate); or
(R)-1-(8-fluoroisochroman-1-yl)-N-methylmethanamine tosylate (Compound 1 Tosylate); or a hydrate or solvate thereof.
[Embodiment B] The salt of Embodiment [B] above, or according to other embodiments of the invention, wherein the salt is a solid form.
[Embodiment C] The salt of Embodiment [A] or [B] above, or according to other embodiments of the invention, wherein the salt is (R)-1-(8-fluoroisochroman-1-yl)-N-methylmethanamine phosphate (Compound 1 Phosphate).
[Embodiment D] The salt of Embodiment [C] above, or according to other embodiments of the invention, wherein Compound 1 Phosphate is crystalline.
[Embodiment E] The salt of Embodiment [D] above, or according to other embodiments of the invention, wherein the salt has characteristic XRPD peaks in terms of 2θ selected from 4.6°±0.2°, 9.1°±0.2°, and 18.2°±0.2°.
[Embodiment F] The salt of Embodiment [D] above, or according to other embodiments of the invention, wherein the salt has at least one characteristic XRPD peak in terms of 2θ selected from 4.6°±0.2°, 9.1°±0.2°, 15.7°±0.2°, 18.2°±0.2°, 22.3°±0.2°, 22.8°±0.2°, and 24.8°±0.2°.
[Embodiment G] The salt of Embodiment [D] above, or according to other embodiments of the invention, wherein the salt has at least two characteristic XRPD peaks in terms of 2θ selected from 4.6°±0.2°, 9.1°±0.2°, 15.7°±0.2°, 18.2°±0.2°, 22.3°±0.2°, 22.8°±0.2°, and 24.8°±0.2°.
[Embodiment H] The salt of Embodiment [D] above, or according to other embodiments of the invention, wherein the salt has at least three characteristic XRPD peaks in terms of 2θ selected from 4.6°±0.2°, 9.1°±0.2°, 15.7°±0.2°, 18.2°±0.2°, 22.3°±0.2°, 22.8°±0.2°, and 24.8°±0.2°.
[Embodiment I] The salt of any one of Embodiments [D] through [H] above, or according to other embodiments of the invention, wherein the salt has an XRPD pattern with characteristic peaks as substantially shown in FIG. 6 (FIG. 6).
[Embodiment J] The salt of any one of Embodiments [D] through [I] above, or according to other embodiments of the invention, wherein the salt has an endotherm peak at a temperature of about 213° C.
[Embodiment K] The salt of any one of Embodiments [D] through [J] above, or according to other embodiments of the invention, wherein the salt has a DSC thermogram substantially as depicted in FIG. 7 (FIG. 7).
[Embodiment L] The salt of any one of Embodiments [D] through [K] above, or according to other embodiments of the invention, wherein the salt has a DVS isotherm substantially as depicted in FIG. 9 (FIG. 9).
[Embodiment M] The salt of Embodiment [A] or [B] above, or according to other embodiments of the invention, wherein the salt is (R)-1-(8-fluoroisochroman-1-yl)-N-methylmethanamine L-tartrate (Compound 1 L-Tartrate).
[Embodiment N] The salt of Embodiment [M] above, or according to other embodiments of the invention, wherein Compound 1 L-tartrate is crystalline.
[Embodiment O] The salt of Embodiment [N] above, or according to other embodiments of the invention, having Form LA.
[Embodiment P] The salt of Embodiment [O] above, or according to other embodiments of the invention, wherein Form LA has characteristic XRPD peaks in terms of 2θ selected from 12.10°±0.2°, 18.10°±0.2°, and 24.2°±0.2°.
[Embodiment Q] The salt of Embodiment [N] or [O] above, or according to other embodiments of the invention, wherein Form LA has an XRPD pattern with characteristic peaks as substantially shown in FIG. 10 (FIG. 10).
[Embodiment R] The salt of Embodiment [N] above, or according to other embodiments of the invention, having Form LB.
[Embodiment S] The salt of Embodiment [R] above, or according to other embodiments of the invention, wherein Form LB has characteristic XRPD peaks in terms of 2θ selected from 18.7°±0.20, 25.0°±0.20, and 31.4°±0.2°.
[Embodiment T] The salt of Embodiment [R] or [S] above, or according to other embodiments of the invention, wherein Form LB has an XRPD pattern with characteristic peaks as substantially shown in FIG. 14 (FIG. 14).
[Embodiment U] The salt of Embodiment [N] above, or according to other embodiments of the invention, having Form LC.
[Embodiment V] The salt of Embodiment [U] above, or according to other embodiments of the invention, wherein Form LC has characteristic XRPD peaks in terms of 2θ selected from 12.2°±0.2°, 16.5°±0.2°, and 24.8°±0.2°.
[Embodiment W] The salt of Embodiment [U] or [V] above, or according to other embodiments of the invention, wherein Form LC has an XRPD pattern with characteristic peaks as substantially shown in FIG. 16 (FIG. 16).
[Embodiment X] The salt of Embodiment [A] or [B] above, or according to other embodiments of the invention, wherein the salt is (R)-1-(8-fluoroisochroman-1-yl)-N-methylmethanamine D-tartrate (Compound 1 D-tartrate).
[Embodiment Y] The salt of Embodiment [X] above, or according to other embodiments of the invention, wherein Compound 1 D-tartrate is crystalline.
[Embodiment Z] The salt of Embodiment [Y] above, or according to other embodiments of the invention, wherein the salt has characteristic XRPD peaks in terms of 2θ selected from 11.9°±0.20, 16.9°±0.2°, and 17.9°±0.2°.
[Embodiment AA] The salt of Embodiment [Y] or [Z] above, or according to other embodiments of the invention, wherein the salt has an XRPD pattern with characteristic peaks as substantially shown in FIG. 20 (FIG. 20).
[Embodiment AB] The salt of Embodiment [A] or [B] above, or according to other embodiments of the invention, wherein the salt is (R)-1-(8-fluoroisochroman-1-yl)-N-methylmethanamine fumarate (Compound 1 L-Fumarate).
[Embodiment AC] The salt of Embodiment [AB] above, or according to other embodiments of the invention, wherein Compound 1 Fumarate is crystalline.
[Embodiment AD] The salt of Embodiment [AC] above, or according to other embodiments of the invention, having Form FA.
[Embodiment AE] The Embodiment [AD] above, or according to other embodiments of the invention, wherein Form FA has characteristic XRPD peaks in terms of 2θ selected from 7.7°±0.2°, 14.2°±0.2°, and 15.2°±0.2°.
[Embodiment AF] The salt of Embodiment [AD] or [AE] above, or according to other embodiments of the invention, wherein Form FA has an XRPD pattern with characteristic peaks as substantially shown in FIG. 22 (FIG. 22).
[Embodiment AG] The salt of Embodiment [AC] above, or according to other embodiments of the invention, having Form FB.
[Embodiment AH] The salt of Embodiment [AG] above, or according to other embodiments of the invention, wherein Form FB has characteristic XRPD peaks in terms of 2θ selected from 6.7°±0.2°, 13.8°±0.2°, and 20.2°±0.2°.
[Embodiment AI] The salt of Embodiment [AG] or [AH] above, or according to other embodiments of the invention, wherein Form FB has an XRPD pattern with characteristic peaks as substantially shown in FIG. 26 (FIG. 26).
[Embodiment AJ] The salt of Embodiment [A] or [B] above, or according to other embodiments of the invention, wherein the salt is (R)-1-(8-fluoroisochroman-1-yl)-N-methylmethanamine citrate (Compound 1 Citrate).
[Embodiment AK] The salt of Embodiment [AJ] above, or according to other embodiments of the invention, wherein Compound 1 Citrate is crystalline.
[Embodiment AL] The Embodiment [AK] above, or according to other embodiments of the invention, wherein the salt has characteristic XRPD peaks in terms of 2θ selected from 6.5⁰ 0.2°, 15.5°±0.2°, and 20.4°±0.2°.
[Embodiment AM] The salt of Embodiment [AK] or [AL] above, or according to other embodiments of the invention, wherein the salt has an XRPD pattern with characteristic peaks as substantially shown in FIG. 30 (FIG. 30).
[Embodiment AN] The salt of Embodiment [A] or [B] above, or according to other embodiments of the invention, wherein the salt is (R)-1-(8-fluoroisochroman-1-yl)-N-methylmethanamine succinate (Compound 1 Succinate).
[Embodiment AO] The salt of Embodiment [AN] above, or according to other embodiments of the invention, wherein Compound 1 Succinate is crystalline.
[Embodiment AP] The salt of Embodiment [AO] above, or according to other embodiments of the invention, wherein the salt has characteristic XRPD peaks in terms of 2θ selected from 6.6°±0.2°, 12.8°±0.2°, and 13.9°±0.2°.
[Embodiment AQ] The salt of Embodiment [AO] or [AP] above, or according to other embodiments of the invention, wherein the salt has an XRPD pattern with characteristic peaks as substantially shown in FIG. 34 (FIG. 34).
[Embodiment AR] The salt of Embodiment [A] or [B] above, or according to other embodiments of the invention, wherein the salt is (R)-1-(8-fluoroisochroman-1-yl)-N-methylmethanamine glutarate (Compound 1 Glutarate).
[Embodiment AS] The salt of Embodiment [AR] above, or according to other embodiments of the invention, wherein Compound 1 Glutarate is crystalline.
[Embodiment AT] The salt of Embodiment [AS] above, or according to other embodiments of the invention, wherein the salt has characteristic XRPD peaks in terms of 2θ selected from 9.1°±0.20, 10.6°±0.20, and 18.2°±0.2°.
[Embodiment AU] The salt of Embodiment [AS] or [AT] above, or according to other embodiments of the invention, wherein the salt has an XRPD pattern with characteristic peaks as substantially shown in FIG. 38 (FIG. 38).
[Embodiment AV] The salt of Embodiment [A] or [B] above, or according to other embodiments of the invention, wherein the salt is (R)-1-(8-fluoroisochroman-1-yl)-N-methylmethanamine L-malate (Compound 1 L-Malate).
[Embodiment AW] The salt of Embodiment [AV] above, or according to other embodiments of the invention, wherein Compound 1 L-Malate is crystalline.
[Embodiment AX] The salt of Embodiment [AW] above, or according to other embodiments of the invention, wherein the salt has characteristic XRPD peaks in terms of 2θ selected from 13.5°±0.2°, 18.8°±0.2°, and 25.2°±0.2°.
[Embodiment AY] The salt of Embodiment [AW] or [AX] above, or according to other embodiments of the invention, wherein the salt has an XRPD pattern with characteristic peaks as substantially shown in FIG. 40 (FIG. 40).
[Embodiment AZ] The salt of Embodiment [A] or [B] above, or according to other embodiments of the invention, wherein the salt is (R)-1-(8-fluoroisochroman-1-yl)-N-methylmethanamine besylate (Compound 1 Besylate).
[Embodiment BA] The salt of Embodiment [AZ] above, or according to other embodiments of the invention, wherein Compound 1 Besylate is crystalline.
[Embodiment BB] The salt of Embodiment [BA] above, or according to other embodiments of the invention, wherein the salt has characteristic XRPD peaks in terms of 2θ selected from 6.0°±0.2°, 12.0°±0.2°, and 24.10°±0.2°.
[Embodiment BC] The salt of Embodiment [BA] or [BB] above, or according to other embodiments of the invention, wherein the salt has an XRPD pattern with characteristic peaks as substantially shown in FIG. 44 (FIG. 44).
[Embodiment BD] The salt of Embodiment [A] above, or according to other embodiments of the invention, wherein the salt is (R)-1-(8-fluoroisochroman-1-yl)-N-methylmethanamine tosylate (Compound 1 Tosylate).
[Embodiment BE] A salt, which is: (R)-1-(8-fluoroisochroman-1-yl)-N-methylmethanamine hydrochloride (Compound 1 Hydrochloride); or a hydrate or solvate thereof, wherein the salt is crystalline and having Form HA or Form HB.
[Embodiment BF] The salt of Embodiment [BE] above, or according to other embodiments of the invention, having Form HA.
[Embodiment BG] The salt of Embodiment [BF] above, or according to other embodiments of the invention, wherein Form HA has characteristic XRPD peaks in terms of 2θ selected from 9.4°±0.20, 11.4±0.20, and 15.10°±0.2°.
[Embodiment BH] The salt of Embodiment [BF] above, or according to other embodiments of the invention, wherein Form HA has characteristic XRPD peaks in terms of 2θ selected from 9.4°±0.20, 11.4±0.20, 15.1°±0.2°, 17.2°±0.2°, and 17.6°±0.2°.
[Embodiment BI] The salt of Embodiment [BF] above, or according to other embodiments of the invention, wherein Form HA has at least one characteristic XRPD peak in terms of 2θ selected from 9.4°±0.2°, 11.4°±0.2°, 14.2°±0.2°, 15.1°±0.2°, 17.2°±0.2°, 17.6°±0.2°, and 27.0°±0.2°.
[Embodiment BJ] The salt of Embodiment [BF] above, or according to other embodiments of the invention, wherein Form HA has at least two characteristic XRPD peaks in terms of 2θ selected from 9.4°±0.2°, 11.4°±0.2°, 14.2°±0.2°, 15.1°±0.2°, 17.2°±0.2°, 17.6°±0.2°, and 27.0°±0.2°.
[Embodiment BK] The salt of Embodiment [BF] above, or according to other embodiments of the invention, wherein Form HA has at least three characteristic XRPD peaks in terms of 2θ selected from 9.4°±0.2°, 11.4°±0.2°, 14.2°±0.2°, 15.1°±0.2°, 17.2°±0.2°, 17.6°±0.2°, and 27.0°±0.2°.
[Embodiment BL] The salt of any one of Embodiments [BE] to [BK] above, or according to other embodiments of the invention, wherein Form HA has an XRPD pattern with characteristic peaks as substantially shown in FIG. 1 (FIG. 1).
[Embodiment BM] The salt of any one of Embodiments [BE] to [BL] above, or according to other embodiments of the invention, wherein Form HA has endotherm peaks at temperatures of about 99° C. and about 187° C.
[Embodiment BN] The salt of any one of Embodiments [BE] to [BM] above, or according to other embodiments of the invention, wherein Form HA has a DSC thermogram substantially as depicted in FIG. 2 (FIG. 2).
[Embodiment BO] The salt of any one of Embodiments [BE] to [BN] above, or according to other embodiments of the invention, wherein Form HA has a DVS isotherm substantially as depicted in FIG. 4 (FIG. 4).
[Embodiment BP] The salt of Embodiment [BE] above, or according to other embodiments of the invention, having Form HB.
[Embodiment BQ] The salt of Embodiment [BP] above, or according to other embodiments of the invention, wherein Form HB has characteristic XRPD peaks in terms of 2θ selected from 8.60°±0.2°, 9.60°±0.2°, and 10.3°±0.2°.
[Embodiment BR] The salt of Embodiment [BP] above, or according to other embodiments of the invention, wherein Form HB has characteristic XRPD peaks in terms of 2θ selected from 8.6°±0.2°, 9.6°±0.2°, 10.3°±0.2°, and 17.3°±0.2°.
[Embodiment BS] The salt of Embodiment [BP] above, or according to other embodiments of the invention, wherein Form HB has at least one characteristic XRPD peak in terms of 2θ selected from 8.6°±0.2°, 9.6°±0.2°, 10.3°±0.2°, 12.6°±0.2°, 14.7°±0.2°, 17.3°±0.2°, and 23.8°±0.2°.
[Embodiment BT] The salt of Embodiment [BP] above, or according to other embodiments of the invention, wherein Form HB has at least two characteristic XRPD peaks in terms of 2θ selected from 8.6°±0.2°, 9.6°±0.2°, 10.3°±0.2°, 12.6°±0.2°, 14.7°±0.2°, 17.3°±0.2°, and 23.8°±0.2°.
[Embodiment BU] The salt of Embodiment [BP] above, or according to other embodiments of the invention, wherein Form HB has at least three characteristic XRPD peaks in terms of 2θ selected from 8.6°±0.2°, 9.6°±0.2°, 10.3°±0.2°, 12.6°±0.2°, 14.7°±0.2°, 17.3°±0.2°, and 23.8°±0.2°.
[Embodiment BV] The salt of any one of Embodiments [BP] to [BU] above, or according to other embodiments of the invention, wherein Form HB has an XRPD pattern with characteristic peaks as substantially shown in FIG. 5 (FIG. 5).
[Embodiment BW] A pharmaceutical composition comprising the salt of any one of Embodiments [A] to [BV] above, or according to other embodiments of the invention, and a pharmaceutically acceptable excipient.
[Embodiment BX] A method for treating a neurological or psychiatric disease or disorder in a subject in need thereof, comprising administering to said subject an effective amount of the salt of any one of Embodiments [A] to [BV] above, or the pharmaceutical composition of [Embodiment BW] above, or according to other embodiments of the invention.
[Embodiment BY] The method according to Embodiment [BX] above, or according to other embodiments of the invention, wherein the neurological or psychiatric disease or disorder is depression, bipolar disorder, pain, schizophrenia, or other psychotic diseases, obsessive compulsive disorder, addiction, social disorder, attention deficit hyperactivity disorder, an anxiety disorder, a movement disorder, epilepsy, autism or cognitive disease or disorder.
[Embodiment BZ] The method according to Embodiment [BX] above, or according to other embodiments of the invention, wherein the neurological or psychiatric disease or disorder is depression.
[Embodiment CA] The method according to Embodiment [BZ] above, or according to other embodiments of the invention, wherein the depression is treatment-resistant depression (TRD), major depressive disorder (MDD), unipolar depression, bipolar depression or depression associated with another disease or disorder.
[Embodiment CB] The method according to Embodiment [BX] above, or according to other embodiments of the invention, wherein said neurological disease or disorder is selected from Alzheimer's disease and Parkinson's disease.
[Embodiment CC] The method according to Embodiment [CB] above, or according to other embodiments of the invention, wherein said Alzheimer's disease is Alzheimer's disease with agitation, Alzheimer's disease with aggression, Alzheimer's disease agitation or Alzheimer's disease with agitation aggression.
[Embodiment CD] A method of treating agitation in a subject in need thereof, comprising administering to said subject an effective amount of the salt of any one of Embodiments [A] to [BV] above, or the pharmaceutical composition of [Embodiment BW] above, or according to other embodiments of the invention.
[Embodiment CE] A method of treating agitation associated with a neurological or psychiatric disease or disorder in a subject in need thereof, comprising administering to said subject an effective amount of the salt of any one of Embodiments [A] to [BV] above, or the pharmaceutical composition of [Embodiment BW] above, or according to other embodiments of the invention.
[Embodiment CF] A process of preparing (R)-1-(8-fluoroisochroman-1-yl)-N-methylmethanamine phosphate (Compound 1 Phosphate), having the structure:

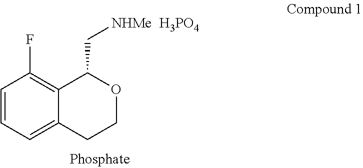

comprising reacting (R)-1-(8-fluoroisochroman-1-yl)-N-methylmethanamine (Compound 1) having the structure:

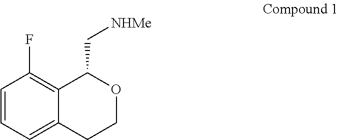 [SMILES: CNC[C@@H]1OCCc2cccc(F)c21]

with phosphoric acid.
[Embodiment CG] The process of Embodiment [CF] above, or according to other embodiments of the invention, wherein the phosphoric acid is an aqueous solution of phosphoric acid.
[Embodiment CH] The process of Embodiment [CF] above, or according to other embodiments of the invention, wherein the aqueous solution of phosphoric acid is an about 80% to about 95% aqueous solution of phosphoric acid by weight.
[Embodiment CI] The process of Embodiment [CF] above, or according to other embodiments of the invention, wherein the aqueous solution of phosphoric acid is an about 87% aqueous solution of phosphoric acid by weight.
[Embodiment CJ] The process of any one of Embodiments [CF] to [CI] above, or according to other embodiments of the invention, wherein the reacting of Compound 1 with phosphoric acid is carried out in the presence of Sla, wherein Sla is a solvent.
[Embodiment CK] The process of Embodiment [CJ] above, or according to other embodiments of the invention, wherein S1a is a mixture of acetonitrile and water.
[Embodiment CL] The process of Embodiment [CJ] above, or according to other embodiments of the invention, wherein S1a is a mixture of acetone and water.
[Embodiment CM] The process of Embodiment [CJ] above, or according to other embodiments of the invention, wherein Sla is a polar aprotic solvent, water, or a mixture thereof.
[Embodiment CN] The process of any one of Embodiments [CF] to [CM] above, or according to other embodiments of the invention, wherein between about 1 and about 5 molar equivalents of phosphoric acid are used per molar equivalent of Compound 1.
[Embodiment CO] The process of any one of Embodiments [CF] to [CN] above, or according to other embodiments of the invention, wherein Compound 1 is prepared by a process comprising reacting (R)-1-(8-fluoroisochroman-1-yl)-N-methylmethanamine dibenzoyl-L-tartrate (Compound 1 dibenzoyl-L-tartrate) having the structure:

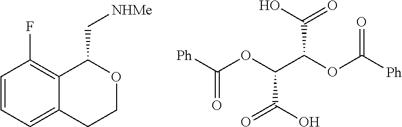 [SMILES: CNC[C@@H]1OCCc2cccc(F)c21 ; O=C(O[C@@H](C(=O)O)[C@@H](OC(=O)c1ccccc1)C(=O)O)c1ccccc1]

Compound 1 dibenzoyl-L-tartrate with B1, wherein B1 is a base.
[Embodiment CP] A process of preparing (R)-1-(8-fluoroisochroman-1-yl)-N-methylmethanamine (Compound 1) comprising reacting (R)-1-(8-fluoroisochroman-1-yl)-N-methylmethanamine dibenzoyl-L-tartrate (Compound 1 dibenzoyl-L-tartrate) having the structure:

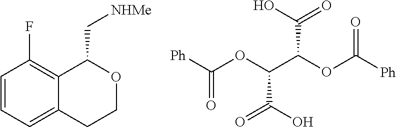 [SMILES: CNC[C@@H]1OCCc2cccc(F)c21 ; O=C(O[C@@H](C(=O)O)[C@@H](OC(=O)c1ccccc1)C(=O)O)c1ccccc1]

Compound 1 dibenzoyl-L-tartrate with B1, wherein B1 is a base.
[Embodiment CQ] The process of Embodiment [CO] or [CP] above, or according to other embodiments of the invention, wherein B1 is an alkali hydroxide base.
[Embodiment CR] The process of Embodiment [CO] or [CP] above, or according to other embodiments of the invention, wherein B1 is potassium hydroxide.
[Embodiment CS] The process of Embodiment [CO] or [CP] above, or according to other embodiments of the invention, wherein B1 is an aqueous solution of potassium hydroxide.
[Embodiment CT] The process of Embodiment [CS] above, or according to other embodiments of the invention, wherein the aqueous solution of potassium hydroxide is an about 10% to about 20% aqueous solution of potassium hydroxide by weight.
[Embodiment CU] The process of Embodiment [CS] above, or according to other embodiments of the invention, wherein the aqueous solution of potassium hydroxide is an about 14% aqueous solution of potassium hydroxide by weight.
[Embodiment CV] The process of any one of Embodiments [CO] to [CU] above, or according to other embodiments of the invention, wherein the reacting of Compound 1 dibenzoyl-L-tartrate and the base is carried out in the presence of S2, wherein S2 is a solvent.
[Embodiment CW] The process of Embodiment [CV] above, or according to other embodiments of the invention, wherein S2 is a polar aprotic solvent.
[Embodiment CX] The process of any one of Embodiments [CO] to [CW] above, or according to other embodiments of the invention, wherein between about 0.5 and about 5 molar equivalents of B1 are used per molar equivalent of Compound 1 dibenzoyl-L-tartrate.
[Embodiment CY] The process of any one of Embodiments [CO] to [CX] above, or according to other embodiments of the invention, wherein the reacting of Compound 1 dibenzoyl-L-tartrate with B1 is further carried out in the presence of sodium chloride.
[Embodiment CZ] The process of any one of Embodiments [CO] to [CY] above, or according to other embodiments of the invention, wherein about 1 to about 10 molar equivalents of sodium chloride are used per molar equivalent of Compound 1 dibenzoyl-L-tartrate.
[Embodiment DA] A process for preparing (R)-1-(8-fluoroisochroman-1-yl)-N-methylmethanamine dibenzoyl-L-tartrate (Compound 1 dibenzoyl-L-tartrate), comprising reacting 1-(8-fluoroisochroman-1-yl)-N-methylmethanamine (Racemic Compound 1) having the structure:

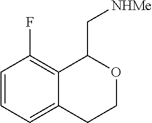 [SMILES: CNCC1OCCc2cccc(F)c21]

### Racemic Compound 1,

with dibenzoyl-L-tartaric acid in the presence of S3, wherein S3 is a solvent.
[Embodiment DB] The process of Embodiment [DA] above, or according to other embodiments of the invention, wherein S3 is a polar protic solvent.
[Embodiment DC] The process of Embodiment [DA] above, or according to other embodiments of the invention, wherein S3 is a mixture of methanol and water.
[Embodiment DD] The process of any one of Embodiments [DA] to [DC] above, or according to other embodiments of the invention, wherein about 1 to about 5 molar equivalents of dibenzoyl-L-tartaric acid are used per molar equivalent of Racemic Compound 1.
[Embodiment DE] The process of any one of Embodiments [DA] to [DD] above, or according to other embodiments of the invention, further comprising precipitating Compound 1 dibenzoyl-L-tartrate from a mixture comprising: Racemic Compound 1, dibenzoyl-L-tartaric acid, and S3.
[Embodiment DF] The process of any one of Embodiments [DA] to [DE] above, or according to other embodiments of the invention, further comprising isolating Compound 1 dibenzoyl-L-tartrate from S3a, wherein S3a is a solvent.
[Embodiment DG] The process of Embodiment [DF] above, or according to other embodiments of the invention, wherein S3a is methanol.
[Embodiment DH] The process of any one of Embodiments [DA] to [DG] above, or according to other embodiments of the invention, wherein 1-(8-fluoroisochroman-1-yl)-N-methylmethanamine (Racemic Compound 1) is prepared by a process comprising hydrogenating a compound of Formula II, having the structure:

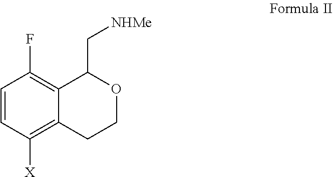

or a salt thereof, wherein X is halo, in the presence of a metal catalyst.
[Embodiment DI] The process of Embodiment [DH] above, or according to other embodiments of the invention, wherein the compound of Formula II is 1-(5-bromo-8-fluoroisochroman-1-yl)-N-methylmethanamine trifluoromethanesulfonate (Compound 2) having the structure:

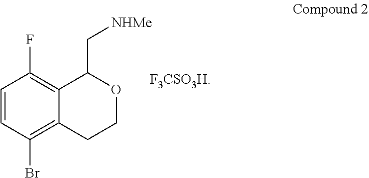 [SMILES: CNCC1OCCc2c(Br)ccc(F)c21]

[Embodiment DJ] The process of Embodiment [DH] or [DI] above, or according to other embodiments of the invention, wherein the metal catalyst is palladium on activated carbon.
[Embodiment DK] The process of any one of Embodiments [DH] to [DJ] above, or according to other embodiments of the invention, wherein the hydrogenating of the compound of Formula II is carried out at a hydrogen pressure of about 2 to about 10 bar.
[Embodiment DL] The process of any one of Embodiments [DH] to [DK] above, or according to other embodiments of the invention, wherein the hydrogenating of the compound of Formula II is carried out at a hydrogen pressure of about 5 bar.
[Embodiment DM] The process of any one of Embodiments [DH] to [DL] above, or according to other embodiments of the invention, wherein the hydrogenating of the compound of Formula II is carried out in the presence of S4, wherein S4 is a solvent.
[Embodiment DN] The process of Embodiment [DM] above, or according to other embodiments of the invention, wherein S4 is a polar protic solvent.
[Embodiment DO] The process of any one of Embodiments [DH] to [DN] above, or according to other embodiments of the invention, wherein the hydrogenating of the compound of Formula II is carried out in the presence of B2, wherein B2 is a base.
[Embodiment DP] The process of Embodiment [DO] above, or according to other embodiments of the invention, wherein B2 is a carbonate base.
[Embodiment DQ] The process of Embodiment [DO] above, or according to other embodiments of the invention, wherein B2 is potassium carbonate.
[Embodiment DR] The process of Embodiment [DO] above, or according to other embodiments of the invention, wherein B2 is an aqueous solution of potassium carbonate.
[Embodiment DS] The process of any one of Embodiments [DH] to [DR] above, or according to other embodiments of the invention, wherein the compound of Formula II is prepared by reacting a compound of Formula III having the structure:

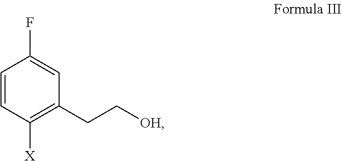

or a salt thereof, wherein X is halo,
with N-methylaminoacetaldehyde dimethylacetal (Compound 4) having the structure:

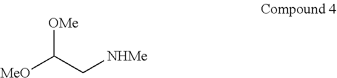

in the presence of A1, wherein A1 is an acid.
[Embodiment DT] The process of Embodiment [DS] above, or according to other embodiments of the invention, wherein the compound of Formula III is 2-(2-bromo-5-fluorophenyl)ethan-1-ol (Compound 3) having the structure:

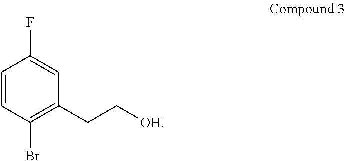 [SMILES: OCCc1cc(F)ccc1Br]

[Embodiment DU] The process of claim Embodiment [DS] or [DT] above, or according to other embodiments of the invention, wherein A1 is trifluoromethanesulfonic acid.
[Embodiment DV] The process of any one of Embodiments [DS] to [DU] above, or according to other embodiments of the invention, wherein the reacting of the compound of Formula III and Compound 4 is carried out in the presence of S5, wherein S5 is a solvent.
[Embodiment DW] The process of Embodiment [DV] above, or according to other embodiments of the invention, wherein S5 is a halogenated solvent.
[Embodiment DX] The process of any one of Embodiments [DS] to [DW] above, or according to other embodiments of the invention, wherein about 1.2 molar equivalents of Compound 4 are used per molar equivalent of the compound of Formula III.
[Embodiment DY] Compound 1 Phosphate prepared by the process of any one of Embodiments [CF] to [CO] above, or according to other embodiments of the invention, wherein Compound 1 Phosphate is crystalline.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1-12. (canceled)

13. A salt, wherein the salt is (R)-1-(8-fluoroisochroman-1-yl)-N-methylmethanamine L-tartrate (Compound 1 L-Tartrate).

14. The salt of claim 13, wherein Compound 1 L-tartrate is a solid form and/or is crystalline.

15. The salt of claim 14 having Form LA, Form LB, or Form LC.

16. The salt of claim 15, wherein;
A) Form LA has:
characteristic XRPD peaks in terms of 2θ selected from 12.1°±0.2°, 18.1°±0.2°, and 24.2°±0.2°;
an XRPD pattern with characteristic peaks as substantially shown in FIG. 10 (FIG. 10);
endotherm peaks at temperatures of about 89° C. and/or about 138° C.;
a DSC thermogram substantially as depicted in FIG. 11 (FIG. 11);
a TGA thermogram substantially as depicted in FIG. 12 (FIG. 12); or
a DVS isotherm substantially as depicted in FIG. 13 (FIG. 13); or
B) Form LB has:
characteristic XRPD peaks in terms of 2θ selected from 18.7°±0.2°, 25.0°±0.2°, and 31.4°±0.2°;
an XRPD pattern with characteristic peaks as substantially shown in FIG. 14 (FIG. 14); or
a DVS isotherm substantially as depicted in FIG. 15 (FIG. 15); or
C) Form LC has:
characteristic XRPD peaks in terms of 2θ selected from 12.2°±0.2°, 16.5°±0.2°, and 24.8°±0.2°;
an XRPD pattern with characteristic peaks as substantially shown in FIG. 16 (FIG. 16);
an endotherm peak at a temperature of about 137° C.;
a DSC thermogram substantially as depicted in FIG. 17 (FIG. 17);
a TGA thermogram substantially as depicted in FIG. 18 (FIG. 18); or
a DVS isotherm substantially as depicted in FIG. 19 (FIG. 19).

17-27. (canceled)

28. A salt, wherein the salt is (R)-1-(8-fluoroisochroman-1-yl)-N-methylmethanamine fumarate (Compound 1 L-Fumarate).

29. The salt of claim 28, wherein Compound 1 Fumarate is a solid form and/or crystalline.

30. The salt of claim 29, having Form FA or Form FB.

31. The salt of claim 30, wherein;
A) Form FA has:
characteristic XRPD peaks in terms of 2θ selected from 7.7°±0.2°, 14.2°±0.2°, and 15.2°±0.2°;
an XRPD pattern with characteristic peaks as substantially shown in FIG. 22 (FIG. 22);
an endotherm peak at a temperature of about 147° C.;
a DSC thermogram substantially as depicted in FIG. 23 (FIG. 23);
a TGA thermogram substantially as depicted in FIG. 24 (FIG. 24); or
a DVS isotherm substantially as depicted in FIG. 25 (FIG. 25); or
B) Form FB has:
characteristic XRPD peaks in terms of 2θ selected from 6.7°±0.2°, 13.8°±0.2°, and 20.2°±0.2°;
an XRPD pattern with characteristic peaks as substantially shown in FIG. 26 (FIG. 26);
an endotherm peak at a temperature of about 96° C., about 139° C., and/or about 146° C.;
a DSC thermogram substantially as depicted in FIG. 27 (FIG. 27);
a TGA thermogram substantially as depicted in FIG. 28 (FIG. 28); or
a DVS isotherm substantially as depicted in FIG. 29 (FIG. 29).

32-35. (canceled)

36. A salt, wherein the salt is (R)-1-(8-fluoroisochroman-1-yl)-N-methylmethanamine citrate (Compound 1 Citrate).

37. The salt of claim 36, wherein Compound 1 Citrate is a solid form and/or crystalline.

38. The salt of claim 37, wherein the salt has:
characteristic XRPD peaks in terms of 2θ selected from 6.5°±0.2°, 15.5°±0.2°, and 20.4°±0.2°;
an XRPD pattern with characteristic peaks as substantially shown in FIG. 30 (FIG. 30):
an endotherm peak at a temperature of about 142° C.:
a DSC thermogram substantially as depicted in FIG. 31 (FIG. 31):
a TGA thermogram substantially as depicted in FIG. 32 (FIG. 32); or
a DVS isotherm substantially as depicted in FIG. 33 (FIG. 33).

39-74. (canceled)

75. A pharmaceutical composition comprising the salt of claim 13 and a pharmaceutically acceptable excipient.

76. A method for treating a neurological or psychiatric disease or disorder in a subject in need thereof, comprising administering to said subject an effective amount of the salt of claim 13.

77-81. (canceled)

82. A method of treating agitation in a subject in need thereof, comprising administering to said subject an effective amount of the salt of claim 13; or a method of treating agitation associated with a neurological or psychiatric disease or disorder in a subject in need thereof, comprising administering to said subject an effective amount of the salt of claim 13.

83-129. (canceled)

130. A pharmaceutical composition comprising the salt of claim 28 and a pharmaceutically acceptable excipient.

131. A method for treating a neurological or psychiatric disease or disorder in a subject in need thereof, comprising administering to said subject an effective amount of the salt of claim 28.

132. A pharmaceutical composition comprising the salt of claim 36 and a pharmaceutically acceptable excipient.

133. A method for treating a neurological or psychiatric disease or disorder in a subject in need thereof, comprising administering to said subject an effective amount of the salt of claim 36.
